SEQUENCE LISTING (298 sequences)

SEQ ID NO: 1 - protein, 193 aa, Mus musculus
QLVPPYRIENQTCWDQDKEYYEPMHDVCCSRCPPGEFVFAVCSRSQDTVCKTCPHNSYNEHWNHLSTCQLCRPCDIVLGFEEVAPCTSDRKAECRCQPGMSCVYLDNECVHCEEERLVLCQPGTEAEVTDEIMDTDVNCVPCKPGHFQNTSSPRARCQPHTRCEIQGLVEAAPGTSYSDTICKNPPEPGAMLL

SEQ ID NO: 2 - protein, 197 aa, Homo sapiens
QAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLM

SEQ ID NO: 3 - protein, 116 aa, synthetic construct
QSVEESGGRLVTPGTPLTLTCTVSGFSLSNYAMSWVRQAPGKGLEWIGTITTGGSTYYASWAKGRFTISKTSSTTVDLKMTSLTIEDTATYFCARVDSGWRTGGIWGPGTLVTVSL

SEQ ID NO: 4 - protein, 110 aa, synthetic construct
AYDMTQTPASVEVAVGGTVTINCQASQNIYNNLAWFQQKPGQPPKLLIYDTSDLASGVPSRFKGSGSGTEYTLTISGVECADAATYYCQQGFTASGVDYTFGGGTEVVVK

SEQ ID NO: 5 - protein, 5 aa, synthetic construct
NYAMS

SEQ ID NO: 6 - protein, 14 aa, synthetic construct
TITTGGSTYYASWA

SEQ ID NO: 7 - protein, 10 aa, synthetic construct
VDSGWRTGGI

SEQ ID NO: 8 - protein, 11 aa, synthetic construct
QASQNIYNNLA

SEQ ID NO: 9 - protein, 7 aa, synthetic construct
DTSDLAS

SEQ ID NO: 10 - protein, 12 aa, synthetic construct
QQGFTASGVDYT

SEQ ID NO: 11 - protein, 121 aa, synthetic construct
QSLEESGGDLVKPGGTLTLTCTASGFSFSNIYWICWVRQAPGKGLEWVACIDGGSSGTTYYASWAKGRFTISKTSSTTVTLQMTSLTAADTATYFCAREFVDDANVYFDLWGPGTLVTVSS

SEQ ID NO: 12 - protein, 111 aa, synthetic construct
AQVLTQTASPVSAAVGGTVTINCQSSQSVASNYLSWYQQKSGQPPKLLIYSASTLASGVPSRFKGSGSGTQFTLTISDLECDDAATYYCAGAYSDSSDIWAFGGGTELVVR

SEQ ID NO: 13 - protein, 5 aa, synthetic construct
IYWIC

SEQ ID NO: 14 - protein, 16 aa, synthetic construct
CIDGGSSGTTYYASWA

SEQ ID NO: 15 - protein, 12 aa, synthetic construct
EFVDDANVYFDL

SEQ ID NO: 16 - protein, 12 aa, synthetic construct
QSSQSVASNYLS

SEQ ID NO: 17 - protein, 7 aa, synthetic construct
SASTLAS

SEQ ID NO: 18 - protein, 12 aa, synthetic construct
AGAYSDSSDIWA

SEQ ID NO: 19 - protein, 119 aa, synthetic construct
QSLEESGGDLVKPGASLTLTCTASGFSFSGYFFMCWVRQAPGKGLEWSACIYAGSSGNTYSASWAKGRFTISKTSSTTVTLQMTSLTAADTATYFCARLDQNAAYVDLWGPGTLVTVSS

SEQ ID NO: 20 - protein, 110 aa, synthetic construct
AFALTQTPASVEAAVGGTVTIKCQASQTFSSYLSWYQQKPGQPPKLLIYRASTLASGVPSRFSGSGFGTEFTLTISDLECADAATYFCQSYYNSINIDFTFGGGTEVVVR

SEQ ID NO: 21 - protein, 5 aa, synthetic construct
YFFMC

SEQ ID NO: 22 - protein, 16 aa, synthetic construct
CIYAGSSGNTYSASWA

SEQ ID NO: 23 - protein, 10 aa, synthetic construct
LDQNAAYVDL

SEQ ID NO: 24 - protein, 11 aa, synthetic construct
QASQTFSSYLS

SEQ ID NO: 25 - protein, 7 aa, synthetic construct
RASTLAS

SEQ ID NO: 26 - protein, 12 aa, synthetic construct
QSYYNSINIDFT

SEQ ID NO: 27 - protein, 123 aa, synthetic construct
QSLEESGGDLVEPGASLTLTCTASGIDFSSYYYMCWVRQAPGKGLELSACIYDVSGSTWYASGAKGRFTISKTSSTTVTLQMTSLTAADTATYFCARGYDSGSDYYYYGMDLWGPGTLVTVSS

SEQ ID NO: 28 - protein, 108 aa, synthetic construct
ALVMTQXASPVSAAVGDTVTINCQASQSIGSNLAWFQQKPGQPPKLLIYAASNLAFGVPSRFSGSRFGTEYTLTISGVQCEDAATYYCLGTYSSSESAFGGGTELEIL

SEQ ID NO: 29 - protein, 5 aa, synthetic construct
YYYMC

SEQ ID NO: 30 - protein, 15 aa, synthetic construct
CIYDVSGSTWYASGA

SEQ ID NO: 31 - protein, 15 aa, synthetic construct
GYDSGSDYYYYGMDL

SEQ ID NO: 32 - protein, 11 aa, synthetic construct
QASQSIGSNLA

SEQ ID NO: 33 - protein, 7 aa, synthetic construct
AASNLAF

SEQ ID NO: 34 - protein, 10 aa, synthetic construct
LGTYSSSESA

SEQ ID NO: 35 - protein, 116 aa, synthetic construct
QSLEESGGRLVTPGTPLTLTCTVSGFTISNYLMCWVRQAPGKGLEYIGIINTGSRTYYASWAKGRFTISKTSTTVDLKITSPTTEDTATYFCARSYGSDIDAFAPWGPGTLVTVSS

SEQ ID NO: 36 - protein, 113 aa, synthetic construct
AQVLTQTASSVXAAVGGTVTINCQASQSVYGNNYLSWYQQKPGQPPKLLIYQASVLAFGVPSRFKGSGFGKQFTLTISGVQCEDAATYYCAGGYVCSSFDCSAFGGGTEVVVK

SEQ ID NO: 37 - protein, 5 aa, synthetic construct
NYLMC

SEQ ID NO: 38 - protein, 14 aa, synthetic construct
IINTGSRTYYASWA

SEQ ID NO: 39 - protein, 11 aa, synthetic construct
SYGSDIDAFAP

SEQ ID NO: 40 - protein, 13 aa, synthetic construct
QASQSVYGNNYLS

SEQ ID NO: 41 - protein, 7 aa, synthetic construct
QASVLAF

SEQ ID NO: 42 - protein, 13 aa, synthetic construct
AGGYVCSSFDCSA

SEQ ID NO: 43 - protein, 117 aa, synthetic construct
QSLQESGGGLFQPGGSLALTCTSSGFSLTDNYVMCWVRQAPGKGLEWIACIITGSSGGTYYASWAKGRFTISKTSSTTLTLQMTSLTAADTATYFCARSGWELFNLWGPGTLVTVSS

SEQ ID NO: 44 - protein, 111 aa, synthetic construct
AIVMTQTPSSTSAAVQGTVTISCQASESLISNYLSWYQQKPGQPPKLLIYDASTLASGVPSRFKGSGSGTQFTLTITNVQREDAATYYCAGYKTSSDDGWAFGAGTNVEIK

SEQ ID NO: 45 - protein, 5 aa, synthetic construct
NYVMC

SEQ ID NO: 46 - protein, 16 aa, synthetic construct
CIITGSSGGTYYASWA

SEQ ID NO: 47 - protein, 8 aa, synthetic construct
SGWELFNL

SEQ ID NO: 48 - protein, 12 aa, synthetic construct
QASESLISNYLS

SEQ ID NO: 49 - protein, 7 aa, synthetic construct
DASTLAS

SEQ ID NO: 50 - protein, 12 aa, synthetic construct
AGYKTSSDDGWA

SEQ ID NO: 51 - protein, 117 aa, synthetic construct
QSVEESGGRLVTPGTPLTLTCTASGFSLSGYYLSWLRQAPGKGLEWIGSIWNDNTYYANWARGRFTISKTSTTVDLKITSPTTEDTATYFCARDAAYGGVGGAFDPWGPGTLVTVSS

SEQ ID NO: 52 - protein, 111 aa, synthetic construct
AAVMTQTPSSVSAAVGGTITINCQSSPSVYSNYLSWFQQKPGQPPKLLIYGASTLASGVPSRFKGSGSGLQFTLSISDVQCDDAATYYCAGGYSGTSDTFAFGGGTEVVVE

SEQ ID NO: 53 - protein, 5 aa, synthetic construct
GYYLS

SEQ ID NO: 54 - protein, 13 aa, synthetic construct
SIWNDNTYYANWA

SEQ ID NO: 55 - protein, 13 aa, synthetic construct
DAAYGGVGGAFDP

SEQ ID NO: 56 - protein, 12 aa, synthetic construct
QSSPSVYSNYLS

SEQ ID NO: 57 - protein, 7 aa, synthetic construct
GASTLAS

SEQ ID NO: 58 - protein, 12 aa, synthetic construct
AGGYSGTSDTFA

SEQ ID NO: 59 - protein, 118 aa, synthetic construct
QSLEESGGDLVKPGASLTLTCTASGFDFSSVAMCWVRQAPGKGLEWIGCIYPISATTYYPSWAKGRFTISKTSSTTVTLQMTSLTAADTATYFCATYNGAPYYYINLWGPGTLVTVSS

SEQ ID NO: 60 - protein, 110 aa, synthetic construct
DVVMPQTPSSTSTAVGGTVTIKCQASQNIGNYLAWYQQKPGQPPKLLIYYAATLASGVPSRFKGGRSGTQFTLTISDVQREDAATYYCLGGTASGSSRTAFGGGTELEIL

SEQ ID NO: 61 - protein, 5 aa, synthetic construct
SVAMC

SEQ ID NO: 62 - protein, 15 aa, synthetic construct
CIYPISATTYYPSWA

SEQ ID NO: 63 - protein, 11 aa, synthetic construct
YNGAPYYYINL

SEQ ID NO: 64 - protein, 11 aa, synthetic construct
QASQNIGNYLA

SEQ ID NO: 65 - protein, 7 aa, synthetic construct
YAATLAS

SEQ ID NO: 66 - protein, 12 aa, synthetic construct
LGGTASGSSRTA

SEQ ID NO: 67 - protein, 124 aa, synthetic construct
QEQVVESGGDLVQPAGSLTLTCTAXGFSLSSAYWICWVRQAPGKGLEWIACIYTGTSGSSDYASWAKGRFTISKTSSTTVTLQMTSLTAADTATYFCARGLLGYGDFGYVFKLWGPGTLVTVSS

SEQ ID NO: 68 - protein, 113 aa, synthetic construct
ADIVMTQTPASVEAAVGGTVTIKCQASQNIYSNLLAWYQQKPGQPPKLLIYRASTLEFGVPSRFKGSGFGTEFTLTISDLECADAATYYCQSYYYYSGSSVNDFGGGTEVVVK

SEQ ID NO: 69 - protein, 5 aa, synthetic construct
AYWIC

SEQ ID NO: 70 - protein, 17 aa, synthetic construct
CIYTGTSGSSDYASWAK

SEQ ID NO: 71 - protein, 14 aa, synthetic construct
GLLGYGDFGYVFKL

SEQ ID NO: 72 - protein, 12 aa, synthetic construct
QASQNIYSNLLA

SEQ ID NO: 73 - protein, 7 aa, synthetic construct
RASTLEF

SEQ ID NO: 74 - protein, 13 aa, synthetic construct
QSYYYYSGSSVND

SEQ ID NO: 75 - protein, 118 aa, synthetic construct
QSXEESGGRLVTPGGSLTLTCTVSGIDLNADHMFWVRQSPKKGLEWIGIIGNNGNTYYASWAKGRFTISKTSTTVSLQVSSPTTEDTATYFCARPIQWLAGGYYFNMWGPGTLVTVSL

SEQ ID NO: 76 - protein, 106 aa, synthetic construct
AIEMTQFPPSLFAFVGDTVTINCLGSENVYSAVAWYQQKPGKPPTLLIYGASNLEFGVPPRFSGSGFGTDYTLTIGGVQAEDAATYVCQGYSNTPTFGAGTKVEIK

SEQ ID NO: 77 - protein, 5 aa, synthetic construct
ADHMF

SEQ ID NO: 78 - protein, 14 aa, synthetic construct
IIGNNGNTYYASWA

SEQ ID NO: 79 - protein, 13 aa, synthetic construct
PIQWLAGGYYFNM

SEQ ID NO: 80 - protein, 11 aa, synthetic construct
LGSENVYSAVA

SEQ ID NO: 81 - protein, 7 aa, synthetic construct
GASNLEF

SEQ ID NO: 82 - protein, 8 aa, synthetic construct
QGYSNTPT

SEQ ID NO: 83 - protein, 114 aa, synthetic construct
QSVEESGGRLVTPGGSLTLTCTVSGFSLSNYGMGWVRQAPGKGLEYIGIINAYNTIYYASWAKGRFTISKTSTTVTLKMTSLTAADTATYFCARNLGWSSINVWGPGTLVTVSL

SEQ ID NO: 84 - protein, 111 aa, synthetic construct
AVVMTQTPSSTSAVVGGTVTIKCQASRSIGSSLSWYQQKPGQPPKLLIYETSELTSGVPSRFSGSGSGTDYTLTISGVQCDDAATYYCLGYFGNTDDGAAFGGGTDLEILC

SEQ ID NO: 85 - protein, 5 aa, synthetic construct
NYGMG

SEQ ID NO: 86 - protein, 14 aa, synthetic construct
IINAYNTIYYASWA

SEQ ID NO: 87 - protein, 9 aa, synthetic construct
NLGWSSINV

SEQ ID NO: 88 - protein, 11 aa, synthetic construct
QASRSIGSSLS

SEQ ID NO: 89 - protein, 7 aa, synthetic construct
ETSELTS

SEQ ID NO: 90 - protein, 12 aa, synthetic construct
LGYFGNTDDGAA

SEQ ID NO: 91 - protein, 117 aa, synthetic construct
QSXEESGGRLVTPGTPLTLTCTAFGFSLSGYYLSWVRQAPGKGLEWIGSIWNDNIYYATWARGRFTISKTSTTVDLKITSPTTEDTATYFCARDAAYGGYGGAFDPWGPGTLVTVSS

SEQ ID NO: 92 - protein, 111 aa, synthetic construct
AAVMTQTPSSVSAAVGGTVTINCQSSPSVYNNYLSWFQQKPGQPPKLLIYGASTLASGVPSRFKGSGSGTQFTLTISDLECDDAATYYCAGGYSGTSDTFAFGGGTEVVVK

SEQ ID NO: 93 - protein, 13 aa, synthetic construct
SIWNDNIYYATWA

SEQ ID NO: 94 - protein, 13 aa, synthetic construct
DAAYGGYGGAFDP

SEQ ID NO: 95 - protein, 12 aa, synthetic construct
QSSPSVYNNYLS

SEQ ID NO: 96 - protein, 118 aa, synthetic construct
QSXEESGGRLVTPGTPLTLTCTAFGFSLSTYAMTWVRQAPGKGLEWIGIIYASGSVVYYATWARGRFTISKTSTTVDLKITSPTTEDTATYFCARGGAGTNSWNLGIWGPGTLVTVSL

SEQ ID NO: 97 - protein, 109 aa, synthetic construct
ALVMTQTPSSTSTAVGDTVTIKCQASQSIDSVYLSWYQQKPGQRPKLLIYEASTLAFGVPSRFKGSGSGKQFTLTISGVQRDDAATYYCLGSYSSNDSAFGGGTELEIL

SEQ ID NO: 98 - protein, 5 aa, synthetic construct
TYAMT

SEQ ID NO: 99 - protein, 15 aa, synthetic construct
IIYASGSVVYYATWA

SEQ ID NO: 100 - protein, 12 aa, synthetic construct
GGAGTNSWNLGI

SEQ ID NO: 101 - protein, 12 aa, synthetic construct
QASQSIDSVYLS

SEQ ID NO: 102 - protein, 7 aa, synthetic construct
EASTLAF

SEQ ID NO: 103 - protein, 10 aa, synthetic construct
LGSYSSNDSA

SEQ ID NO: 104 - protein, 117 aa, synthetic construct
QSXEESGGRLVTPGTPLTLTCTAFGFSLSGYYLSWLRQAPGKGLEWIGSIWNDNTYYANWARGRFTISKTSTTVDLKITSPTTEDTATYFCARDAAYGGVGGAFDPWGPGTLVTVSS

SEQ ID NO: 105 - protein, 111 aa, synthetic construct
AAVMTQTPSSVFAAVGGTITINCQSSPSVYSNYLSWFQQKPGQPPKLLIYGASTLASGVPSRFKGSGSGLQFTLSISDVQCDDAATYYCAGGYSGTSDTFAFGGGTEVVVE

SEQ ID NO: 106 - protein, 108 aa, synthetic construct
QSVEESGGRLVTPGTPLTLTCTASGFSLNDYDWNWVRQAPGEELKWVATIHTYHTTYYANWAKGRFTISRTSTTVDLKMTSPTTEDTATYFCGANNVWGPGTLVTVSL

SEQ ID NO: 107 - protein, 113 aa, synthetic construct
AQVXTQTPSXVXAXVGGTVTINCQASQSVYKNNXLSWFQQKPGQPPKRLIYYVSTLNFGVSSRFKGSGSGTQFTLTISDVRCDDAATYYCLGNYDCRSADCLVFGGGTEVLVK

SEQ ID NO: 108 - protein, 5 aa, synthetic construct
DYDWN

SEQ ID NO: 109 - protein, 14 aa, synthetic construct
TIHTYHTTYYANWA

SEQ ID NO: 110-111 - skipped (2 entries)

SEQ ID NO: 112 - protein, 6 aa, synthetic construct
YVSTLN

SEQ ID NO: 113 - protein, 13 aa, synthetic construct
LGNYDCRSADCLV

SEQ ID NO: 114 - protein, 114 aa, synthetic construct
QSVEESGGRLVTPGTPLTLTCTVFGFSLSNYDMSWVRQAPGKGLEWIGIISTGDIKYYANWARGRFTISKTSTTVDLQITSLTTEDTATYFCARESHWGIENVWGPGTLVTVSL

SEQ ID NO: 115 - protein, 111 aa, synthetic construct
AQVLTQTPSSVSAAVGGTVTINCQSSPSVYRNNLLSWFQQKSGQPPKLLIYQVSELAFGVPSRFSGSGSGTEFTLTISDVQCDDGATYYCAGGYSRNSDRRAFGGGTEVVV

SEQ ID NO: 116 - protein, 5 aa, synthetic construct
NYDMS

SEQ ID NO: 117 - protein, 14 aa, synthetic construct
IISTGDIKYYANWA

SEQ ID NO: 118 - protein, 9 aa, synthetic construct
ESHWGIENV

SEQ ID NO: 119 - protein, 13 aa, synthetic construct
QSSPSVYRNNLLS

SEQ ID NO: 120 - protein, 7 aa, synthetic construct
QVSELAF

SEQ ID NO: 121 - protein, 11 aa, synthetic construct
GGYSRNSDRRA

SEQ ID NO: 122 - protein, 446 aa, synthetic construct
QSVEESGGRLVTPGTPLTLTCTVSGFSLSNYAMSWVRQAPGKGLEWIGTITTGGSTYYASWAKGRFTISKTSSTTVDLKMTSLTIEDTATYFCARVDSGWRTGGIWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 217 aa, synthetic construct
AYDMTQTPASVEVAVGGTVTINCQASQNIYNNLAWFQQKPGQPPKLLIYDTSDLASGVPSRFKGSGSGTEYTLTISGVECADAATYYCQQGFTASGVDYTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 451 aa, synthetic construct
QSLEESGGDLVKPGGTLTLTCTASGFSFSNIYWICWVRQAPGKGLEWVACIDGGSSGTTYYASWAKGRFTISKTSSTTVTLQMTSLTAADTATYFCAREFVDDANVYFDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 218 aa, synthetic construct
AQVLTQTASPVSAAVGGTVTINCQSSQSVASNYLSWYQQKSGQPPKLLIYSASTLASGVPSRFKGSGSGTQFTLTISDLECDDAATYYCAGAYSDSSDIWAFGGGTELVVRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 449 aa, synthetic construct
QSLEESGGDLVKPGASLTLTCTASGFSFSGYFFMCWVRQAPGKGLEWSACIYAGSSGNTYSASWAKGRFTISKTSSTTVTLQMTSLTAADTATYFCARLDQNAAYVDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 217 aa, synthetic construct
AFALTQTPASVEAAVGGTVTIKCQASQTFSSYLSWYQQKPGQPPKLLIYRASTLASGVPSRFSGSGFGTEFTLTISDLECADAATYFCQSYYNSINIDFTFGGGTEVVVRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 453 aa, synthetic construct
QSLEESGGDLVEPGASLTLTCTASGIDFSSYYYMCWVRQAPGKGLELSACIYDVSGSTWYASGAKGRFTISKTSSTTVTLQMTSLTAADTATYFCARGYDSGSDYYYYGMDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 215 aa, synthetic construct
ALVMTQXASPVSAAVGDTVTINCQASQSIGSNLAWFQQKPGQPPKLLIYAASNLAFGVPSRFSGSRFGTEYTLTISGVQCEDAATYYCLGTYSSSESAFGGGTELEILRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 446 aa, synthetic construct
QSLEESGGRLVTPGTPLTLTCTVSGFTISNYLMCWVRQAPGKGLEYIGIINTGSRTYYASWAKGRFTISKTSTTVDLKITSPTTEDTATYFCARSYGSDIDAFAPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 220 aa, synthetic construct
AQVLTQTASSVXAAVGGTVTINCQASQSVYGNNYLSWYQQKPGQPPKLLIYQASVLAFGVPSRFKGSGFGKQFTLTISGVQCEDAATYYCAGGYVCSSFDCSAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 447 aa, synthetic construct
QSLQESGGGLFQPGGSLALTCTSSGFSLTDNYVMCWVRQAPGKGLEWIACIITGSSGGTYYASWAKGRFTISKTSSTTLTLQMTSLTAADTATYFCARSGWELFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 218 aa, synthetic construct
AIVMTQTPSSTSAAVQGTVTISCQASESLISNYLSWYQQKPGQPPKLLIYDASTLASGVPSRFKGSGSGTQFTLTITNVQREDAATYYCAGYKTSSDDGWAFGAGTNVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 447 aa, synthetic construct
QSVEESGGRLVTPGTPLTLTCTASGFSLSGYYLSWLRQAPGKGLEWIGSIWNDNTYYANWARGRFTISKTSTTVDLKITSPTTEDTATYFCARDAAYGGVGGAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 218 aa, synthetic construct
AAVMTQTPSSVSAAVGGTITINCQSSPSVYSNYLSWFQQKPGQPPKLLIYGASTLASGVPSRFKGSGSGLQFTLSISDVQCDDAATYYCAGGYSGTSDTFAFGGGTEVVVERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 448 aa, synthetic construct
QSLEESGGDLVKPGASLTLTCTASGFDFSSVAMCWVRQAPGKGLEWIGCIYPISATTYYPSWAKGRFTISKTSSTTVTLQMTSLTAADTATYFCATYNGAPYYYINLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 217 aa, synthetic construct
DVVMPQTPSSTSTAVGGTVTIKCQASQNIGNYLAWYQQKPGQPPKLLIYYAATLASGVPSRFKGGRSGTQFTLTISDVQREDAATYYCLGGTASGSSRTAFGGGTELEILRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 454 aa, synthetic construct
QEQVVESGGDLVQPAGSLTLTCTAXGFSLSSAYWICWVRQAPGKGLEWIACIYTGTSGSSDYASWAKGRFTISKTSSTTVTLQMTSLTAADTATYFCARGLLGYGDFGYVFKLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 220 aa, synthetic construct
ADIVMTQTPASVEAAVGGTVTIKCQASQNIYSNLLAWYQQKPGQPPKLLIYRASTLEFGVPSRFKGSGFGTEFTLTISDLECADAATYYCQSYYYYSGSSVNDFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 448 aa, synthetic construct
QSXEESGGRLVTPGGSLTLTCTVSGIDLNADHMFWVRQSPKKGLEWIGIIGNNGNTYYASWAKGRFTISKTSTTVSLQVSSPTTEDTATYFCARPIQWLAGGYYFNMWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 213 aa, synthetic construct
AIEMTQFPPSLFAFVGDTVTINCLGSENVYSAVAWYQQKPGKPPTLLIYGASNLEFGVPPRFSGSGFGTDYTLTIGGVQAEDAATYVCQGYSNTPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 444 aa, synthetic construct
QSVEESGGRLVTPGGSLTLTCTVSGFSLSNYGMGWVRQAPGKGLEYIGIINAYNTIYYASWAKGRFTISKTSTTVTLKMTSLTAADTATYFCARNLGWSSINVWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 218 aa, synthetic construct
AVVMTQTPSSTSAVVGGTVTIKCQASRSIGSSLSWYQQKPGQPPKLLIYETSELTSGVPSRFSGSGSGTDYTLTISGVQCDDAATYYCLGYFGNTDDGAAFGGGTDLEILCRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 447 aa, synthetic construct
QSXEESGGRLVTPGTPLTLTCTAFGFSLSGYYLSWVRQAPGKGLEWIGSIWNDNIYYATWARGRFTISKTSTTVDLKITSPTTEDTATYFCARDAAYGGYGGAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 218 aa, synthetic construct
AAVMTQTPSSVSAAVGGTVTINCQSSPSVYNNYLSWFQQKPGQPPKLLIYGASTLASGVPSRFKGSGSGTQFTLTISDLECDDAATYYCAGGYSGTSDTFAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 448 aa, synthetic construct
QSXEESGGRLVTPGTPLTLTCTAFGFSLSTYAMTWVRQAPGKGLEWIGIIYASGSVVYYATWARGRFTISKTSTTVDLKITSPTTEDTATYFCARGGAGTNSWNLGIWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 216 aa, synthetic construct
ALVMTQTPSSTSTAVGDTVTIKCQASQSIDSVYLSWYQQKPGQRPKLLIYEASTLAFGVPSRFKGSGSGKQFTLTISGVQRDDAATYYCLGSYSSNDSAFGGGTELEILRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 447 aa, synthetic construct
QSXEESGGRLVTPGTPLTLTCTAFGFSLSGYYLSWLRQAPGKGLEWIGSIWNDNTYYANWARGRFTISKTSTTVDLKITSPTTEDTATYFCARDAAYGGVGGAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 218 aa, synthetic construct
AAVMTQTPSSVFAAVGGTITINCQSSPSVYSNYLSWFQQKPGQPPKLLIYGASTLASGVPSRFKGSGSGLQFTLSISDVQCDDAATYYCAGGYSGTSDTFAFGGGTEVVVERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 438 aa, synthetic construct
QSVEESGGRLVTPGTPLTLTCTASGFSLNDYDWNWVRQAPGEELKWVATIHTYHTTYYANWAKGRFTISRTSTTVDLKMTSPTTEDTATYFCGANNVWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 220 aa, synthetic construct
AQVXTQTPSXVXAXVGGTVTINCQASQSVYKNNXLSWFQQKPGQPPKRLIYYVSTLNFGVSSRFKGSGSGTQFTLTISDVRCDDAATYYCLGNYDCRSADCLVFGGGTEVLVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 444 aa, synthetic construct
QSVEESGGRLVTPGTPLTLTCTVFGFSLSNYDMSWVRQAPGKGLEWIGIISTGDIKYYANWARGRFTISKTSTTVDLQITSLTTEDTATYFCARESHWGIENVWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 218 aa, synthetic construct
AQVLTQTPSSVSAAVGGTVTINCQSSPSVYRNNLLSWFQQKSGQPPKLLIYQVSELAFGVPSRFSGSGSGTEFTLTISDVQCDDGATYYCAGGYSRNSDRRAFGGGTEVVVRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - DNA, 1017 bp, synthetic construct
  source 1..1017: mol_type = other DNA
atgctgctgctggtgaccagcctgctgctgtgcgagctgccacatcctgccttcctgctgatccctgatgtgcagatgacccagagccccagctacctggccgccagccccggcgaatctgtgtcaattaggtgcaaggccagcaagagcatctccaacaacctggcctggtaccaggaaaaacccggcaaggccaacaagctgctgatccatagcggctctactctgcagtccgggaccccaagccgcttcagtgggtccggcagtggaacagatttcaccctgaccatccggagcctggagtttgaggactttgccgtgtattactgccagcagtacaacgaatacccttacactttcggcgccggcaccaagctggagctgaaaagagctgacgccggcggcgggggaagtggaggcggaggcagcggcggcggcggttcccaggtgcagctgaaggagtcaggaccaggcctggtgcaaccaagccagacactgtccctgacatgtacagtgtccggcttcagcctgaccacatactctgtgcactgggtgagacagcacagcggcaaatcactggagtggatggggagaatgtggaccgatggagacacaagctacaactccgccttcacaagcagactgtccatctctagggataccagcaagagccaggtgttcctgaagatgaattctctgcagaccgaagatacaggaacctactactgcgcccggggctattggtacttcgacttctggggacctggaaccatggtgactgtgagcagcgctgagaccacaggcggcggcatcgaattcatgtatccacctccatacctggataacgaaaggtcaaatggaaccattatccatatcaaggagaagcacctgtgtcacacacagagttccccaaagctgttttgggccctggtggtggtggccggcgtgctgttttgttacgggctgctggtgaccgttgctctgtgcgtcatttggactaattccaggcgcaattga

SEQ ID NO: 155 - protein, 338 aa, synthetic construct
MLLLVTSLLLCELPHPAFLLIPDVQMTQSPSYLAASPGESVSIRCKASKSISNNLAWYQEKPGKANKLLIHSGSTLQSGTPSRFSGSGSGTDFTLTIRSLEFEDFAVYYCQQYNEYPYTFGAGTKLELKRADAGGGGSGGGGSGGGGSQVQLKESGPGLVQPSQTLSLTCTVSGFSLTTYSVHWVRQHSGKSLEWMGRMWTDGDTSYNSAFTSRLSISRDTSKSQVFLKMNSLQTEDTGTYYCARGYWYFDFWGPGTMVTVSSAETTGGGIEFMYPPPYLDNERSNGTIIHIKEKHLCHTQSSPKLFWALVVVAGVLFCYGLLVTVALCVIWTNSRRN

SEQ ID NO: 156 - protein, 116 aa, synthetic construct; identical to SEQ ID NO: 3

SEQ ID NO: 157 - protein, 110 aa, synthetic construct
AYDMTQTPASVEVAVGGTVTINCQASQNIYNNLAWFQQKPGQPPKLLIYDTSDLASGVPSRFKGSGSGTEYTLTVSGVECADAATYYCQQGFTASGVDYTFGGGTEVVVK

SEQ ID NO: 158 - protein, 123 aa, synthetic construct; identical to SEQ ID NO: 27

SEQ ID NO: 159 - protein, 108 aa, synthetic construct
DIVMTQTASPVSAAVGDTFTINCQASQSIGSNLAWFQQKPGQPPKLLIYAASNLASGVPSRFSGSRSGTEYTLTISGVQCEDAATYYCLGTYSSSESAFGGGTELEIL

SEQ ID NO: 160 - protein, 116 aa, synthetic construct
QSLEESGGRLVTPGTPLTLTCTVSGFTISNYLMCWVRQAPGKGLEYIGIINTGSRTYYASWAKGRFTISKTSTTVDLKITSPTTEDTATYFCARSYGSDIDAIAPWGPGTLVTVSS

SEQ ID NO: 161 - protein, 114 aa, synthetic construct
FAQVLTQTASSVSAAVGGTVTINCQASQSVYGNNYLSWYQQKPGQPPKLLIYQASVLASGVSSRFKGSGSGKQFTLTISGVQCEDAATYYCAGGYVCSSFDCSAFGGGTEVVVK

SEQ ID NO: 162-164 - skipped (3 entries)

SEQ ID NO: 165 - protein, 35 aa, synthetic construct
IEFMYPPPYLDNERSNGTIIHIKEKHLCHTQSSPK

SEQ ID NO: 166 - protein, 39 aa, synthetic construct
IEVMYPPPYLDNEKSNGTIIHVKGKHLCPSPLFPGPSKP

SEQ ID NO: 167 - protein, 48 aa, synthetic construct
AKPTTTPAPRPPTPAPTIASQPLSLRPEACRPAAGGAVHTRGLDFACD

SEQ ID NO: 168 - protein, 45 aa, synthetic construct
TTTKPVLRTPSPVHPTGTSQPQRPEDCRPRGSVKGTGLDFACDIY

SEQ ID NO: 169 - protein, 28 aa, synthetic construct
LFWALVVVAGVLFCYGLLVTVALCVIWT

SEQ ID NO: 170 - protein, 28 aa, synthetic construct
MFWVLVVVGGVLACYSLLVTVAFIIFWV

SEQ ID NO: 171 - protein, 22 aa, synthetic construct
IWAPLAGICVALLLSLIITLIC

SEQ ID NO: 172 - protein, 21 aa, synthetic construct
IYIWAPLAGTCGVLLLSLVIT

SEQ ID NO: 173 - protein, 23 aa, synthetic construct
IYIWAPLAGTCGVLLLSLVITLY

SEQ ID NO: 174 - protein, 24 aa, synthetic construct
IYIWAPLAGTCGVLLLSLVITLYC

SEQ ID NO: 175 - protein, 15 aa, synthetic construct
GGGGSGGGGSGGGGS

SEQ ID NO: 176 - protein, 10 aa, synthetic construct
GGGSSGGGSG

SEQ ID NO: 177 - protein, 7 aa, synthetic construct
GGSAGGG

SEQ ID NO: 178 - protein, 23 aa, synthetic construct
GGSAGPAENLYFQGGGGSGGGSG

SEQ ID NO: 179 - protein, 114 aa, synthetic construct
QSLEESGGRLVTPGGSLTLTCTVSGFSLSSRSMGWVRQAPEKGLEWIGIIGGSGDVRYATWARGRFTISSTSTTVDLKMTSLTAEDTATYFCGRYNGAGAIDIWGPGTLVTVSF

SEQ ID NO: 180 - protein, 109 aa, synthetic construct
DPVLTQTPSSVSAVVGGTVTINCQSSQSVYSRGCSWYQQKPGQPPKLLIYQASTLASGVSSRFSGSGYGTQFALTISGVQCDDAATYYCLGGYSGSIYVFGGGTEVVVK

SEQ ID NO: 181 - protein, 5 aa, synthetic construct
SRSMG

SEQ ID NO: 182 - protein, 14 aa, synthetic construct
IIGGSGDVRYATWA

SEQ ID NO: 183 - protein, 9 aa, synthetic construct
YNGAGAIDI

SEQ ID NO: 184 - protein, 12 aa, synthetic construct
QSSQSVYSRGCS

SEQ ID NO: 185 - protein, 7 aa, synthetic construct
QASTLAS

SEQ ID NO: 186 - protein, 10 aa, synthetic construct
LGGYSGSIYV

SEQ ID NO: 187 - protein, 116 aa, synthetic construct
QSVEESGGRLVTPGTPLTLTCTVSGIDLSSYAMGWVRQAPGEGLEWIGLIDAAGNTYYATWAKGRFTISKTSSTTVDLKMTSLTTEDTATYFCARESSSDWYMDIWGPGTLVTVSL

SEQ ID NO: 188 - protein, 110 aa, synthetic construct
ARVLTQTPSPVSAAVGDTVTINCQASQSVYGANQLSWYQQKPGQPPKLLIYQASKLASGVPSRLKGSGSGTQFTLTISEVQCDDAATYFCQGYYSGGIYTFGGGTEVVVK

SEQ ID NO: 189 - protein, 5 aa, synthetic construct
SYAMG

SEQ ID NO: 190 - protein, 14 aa, synthetic construct
LIDAAGNTYYATWA

SEQ ID NO: 191 - protein, 10 aa, synthetic construct
ESSSDWYMDI

SEQ ID NO: 192 - protein, 13 aa, synthetic construct
QASQSVYGANQLS

SEQ ID NO: 193 - protein, 7 aa, synthetic construct
QASKLAS

SEQ ID NO: 194 - protein, 10 aa, synthetic construct
QGYYSGGIYT

SEQ ID NO: 195 - protein, 114 aa, synthetic construct
QSLEESGGRLVTPGGSLTLTCTASGSDISPYTISWVRQAPEKGLEWIGIIGKSGGPYYATWAKGPFTISKTSTTVALKMTSLTAEDTATYFCGRYNGAGAMDIWGPGTLVTVSL

SEQ ID NO: 196 - protein, 109 aa, synthetic construct
DPVLTQTPSSVFALVGGTVSINCQSSQSVYSMGCSWYQQKPGQPPKLLIYQASTLASGVSSRFKGSGSGTQFTLTISDVQCDDAATYYCLGGYSGNIYVFGGGTEVVVK

SEQ ID NO: 197 - protein, 5 aa, synthetic construct
PYTIS

SEQ ID NO: 198 - protein, 14 aa, synthetic construct
IIGKSGGPYYATWA

SEQ ID NO: 199 - protein, 9 aa, synthetic construct
YNGAGAMDI

SEQ ID NO: 200 - protein, 12 aa, synthetic construct
QSSQSVYSMGCS

SEQ ID NO: 201 - protein, 7 aa, synthetic construct
QASTLAS

SEQ ID NO: 202 - protein, 10 aa, synthetic construct
LGGYSGNIYV

SEQ ID NO: 203 - protein, 121 aa, synthetic construct
QSVEESGGRLVTPGTPLTLTCTISGFSLSSYDMSWVRQAPGEGLEWIGTISTGGSTYYASWAKGRFTISKTSSTTVDLKITSPTIEDTATYFCARDRGRSSYYTFGAFDPWGPGTLVTVSS

SEQ ID NO: 204 - protein, 108 aa, synthetic construct
ALVMTQTPSSTSTAVGDTVTIKCQASQSITSYLSWFQQKPGQPPKLLIYLASTLASGVPSRFKGSGSGTEFTLTISGVQREDAATYYCLGSYSHSDTAFGGGTELELL

SEQ ID NO: 205 - protein, 5 aa, synthetic construct
SYDMS

SEQ ID NO: 206 - protein, 14 aa, synthetic construct
TISTGGSTYYASWA

SEQ ID NO: 207 - protein, 15 aa, synthetic construct
DRGRSSYYTFGAFDP

SEQ ID NO: 208 - protein, 11 aa, synthetic construct
QASQSITSYLS

SEQ ID NO: 209 - protein, 7 aa, synthetic construct
LASTLAS

SEQ ID NO: 210 - protein, 10 aa, synthetic construct
LGSYSHSDTA

SEQ ID NO: 211 - protein, 118 aa, synthetic construct
QLKESGGGLVQPGGSLKLSCKASGFDFSSYYMSWVRQAPGKGLEWIGWISSGGSTWYASWVNGRFTISSHNAQNTLYLQLNSLTAADTATYFCARDRDSSAWGDFNLWGPGTLVTVSS

SEQ ID NO: 212 - protein, 111 aa, synthetic construct
DPVMTQTPASVFEPVGGTVTINCQASESISNYLSWYQQKPGQPPKLLIYGASTLEFGVPSRFSGSGFGTEFSLTISGVQCDDAATYYCQGGYYSSSGTFFAFGGGTEVVVK

SEQ ID NO: 213 - protein, 5 aa, synthetic construct
SYYMS

SEQ ID NO: 214 - protein, 16 aa, synthetic construct
WISSGGSTWYASWVNG

SEQ ID NO: 215 - protein, 12 aa, synthetic construct
DRDSSAWGDFNL

SEQ ID NO: 216 - protein, 11 aa, synthetic construct
QASESISNYLS

SEQ ID NO: 217 - protein, 7 aa, synthetic construct
GASTLEF

SEQ ID NO: 218 - protein, 13 aa, synthetic construct
QGGYYSSSGTFFA

SEQ ID NO: 219 - protein, 114 aa, synthetic construct
QSXEESGGRLVTPGTPLTLTCTASGXSLSNYQMGWVRQAPGEGLEWIGIISSSGSAYYASWVNGRFTISKTSTTVDLKVTSLTTEDTATYFCARYLNNDYYDIWGPGTLVTVSL

SEQ ID NO: 220 - protein, 110 aa, synthetic construct
AQVLTQTPSSVSAAVGGTVTINCQASQSISSYLAWYQQKRGQPPKLLIYYASNLAFGVSSRFKGSGFGTEYTLTISDLECDDAATYYCQQGYSDINVDNTFGGGTEVVVK

SEQ ID NO: 221 - protein, 5 aa, synthetic construct
NYQMG

SEQ ID NO: 222 - protein, 14 aa, synthetic construct
IISSSGSAYYASWV

SEQ ID NO: 223 - protein, 9 aa, synthetic construct
YLNNDYYDI

SEQ ID NO: 224 - protein, 11 aa, synthetic construct
QASQSISSYLA

SEQ ID NO: 225 - protein, 7 aa, synthetic construct
YASNLAF

SEQ ID NO: 226 - protein, 12 aa, synthetic construct
QQGYSDINVDNT

SEQ ID NO: 227 - protein, 116 aa, synthetic construct
QSXEESGGXLVTPGTPLTLTCTVFGFSLSSNAMSWVRQAPGKGLEWIGFINRYGTTYYANWAKGRFTISKTSTTVDLKITSPTIEDTATYFCARADGVEYGYTRIWGPGTLVTVSL

SEQ ID NO: 228 - protein, 110 aa, synthetic construct
ALVMTQTPSSVFAAVGGTVTINCQASQNIYSNLAWYQQKPGQRPNLLIYEASKMESGVPSRFSGSGSGPEYTLTISSVQCDDAATYYCQSGAYSASLDNTFGGGTEVVVK

SEQ ID NO: 229 - protein, 5 aa, synthetic construct
SNAMS

SEQ ID NO: 230 - protein, 14 aa, synthetic construct
FINRYGTTYYANWA

SEQ ID NO: 231 - protein, 11 aa, synthetic construct
ADGVEYGYTRI

SEQ ID NO: 232 - protein, 11 aa, synthetic construct
QASQNIYSNLA

SEQ ID NO: 233 - protein, 7 aa, synthetic construct
EASKMES

SEQ ID NO: 234 - protein, 12 aa, synthetic construct
QSGAYSASLDNT

SEQ ID NO: 235 - protein, 121 aa, synthetic construct
QSVEESGGXLVTPGTPLTLTCTIFGFSLSSYDMSWVRQAPGEGLEWIGTISTGGSTYYASWGKGRFTISKTSSTTVDLKITSPTMEDTATYFCARDRGRSSYYTFGAFDPWGPGTLVTVSS

SEQ ID NO: 236 - protein, 108 aa, synthetic construct; identical to SEQ ID NO: 204

SEQ ID NO: 237 - protein, 5 aa, synthetic construct
SYDMS

SEQ ID NO: 238 - protein, 14 aa, synthetic construct
TISTGGSTYYASWG

SEQ ID NO: 239 - protein, 15 aa, synthetic construct
DRGRSSYYTFGAFDP

SEQ ID NO: 240 - protein, 11 aa, synthetic construct
QASQSITSYLS

SEQ ID NO: 241 - protein, 7 aa, synthetic construct
LASTLAS

SEQ ID NO: 242 - protein, 10 aa, synthetic construct
LGSYSHSDTA

SEQ ID NO: 243 - protein, 113 aa, synthetic construct
QSVEESGGRLVTPGTPLTLTCTVSGFSLSRYGMSWVRQAPGKGLEWIGMISSSGSTYNPNWAKGRFTISKTSTTVDLKITSPTTEDTATYFCAREYWYYFNIWGPGTLVTVSL

SEQ ID NO: 244 - protein, 112 aa, synthetic construct
AQVLTQTPSSVSAAVGGTVTINCQSSQSVYSTNYLAWYQQKPGQPPKLLIYDASSLASGVPSRFSGSGSGTQFTLTISGVQCDDAATYYCQGAYYASGWYHTFGGGTEVVVK

SEQ ID NO: 245 - protein, 5 aa, synthetic construct
RYGMS

SEQ ID NO: 246 - protein, 14 aa, synthetic construct
MISSSGSTYNPNWA

SEQ ID NO: 247 - protein, 8 aa, synthetic construct
EYWYYFNI

SEQ ID NO: 248 - protein, 13 aa, synthetic construct
QSSQSVYSTNYLA

SEQ ID NO: 249 - protein, 7 aa, synthetic construct
DASSLAS

SEQ ID NO: 250 - protein, 12 aa, synthetic construct
QGAYYASGWYHT

SEQ ID NO: 251 - protein, 114 aa, synthetic construct
QSGEESGGRLVTPGTPLTLTCTAFGXSLSNYQMGWVRQAPGEGLEWIGIISSSGSAYYASWVNGRFTISKTSTTVDLKVTSLTTEDTATYFCARYLNNDYYDIWGPGTLVTVSL

SEQ ID NO: 252 - protein, 110 aa, synthetic construct
AQVLTQTPSSVSAAVGGTVTINCQASQSISSYLAWYQQKRGQPPKLLIYYASNLASGVSSRFKGSGFGTEYTLTISDLECDDAATYYCQQGYSDINVDNTFGGGTEVVVK

SEQ ID NO: 253 - protein, 5 aa, synthetic construct
NYQMG

SEQ ID NO: 254 - protein, 14 aa, synthetic construct
IISSSGSAYYASWV

SEQ ID NO: 255 - protein, 9 aa, synthetic construct
YLNNDYYDI

SEQ ID NO: 256 - protein, 11 aa, synthetic construct
QASQSISSYLA

SEQ ID NO: 257 - protein, 7 aa, synthetic construct
YASNLAS

SEQ ID NO: 258 - protein, 12 aa, synthetic construct
QQGYSDINVDNT

SEQ ID NO: 259 - protein, 444 aa, synthetic construct
QSLEESGGRLVTPGGSLTLTCTVSGFSLSSRSMGWVRQAPEKGLEWIGIIGGSGDVRYATWARGRFTISSTSTTVDLKMTSLTAEDTATYFCGRYNGAGAIDIWGPGTLVTVSFASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 216 aa, synthetic construct
DPVLTQTPSSVSAVVGGTVTINCQSSQSVYSRGCSWYQQKPGQPPKLLIYQASTLASGVSSRFSGSGYGTQFALTISGVQCDDAATYYCLGGYSGSIYVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 261 - protein, 446 aa, synthetic construct
QSVEESGGRLVTPGTPLTLTCTVSGIDLSSYAMGWVRQAPGEGLEWIGLIDAAGNTYYATWAKGRFTISKTSSTTVDLKMTSLTTEDTATYFCARESSSDWYMDIWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - protein, 217 aa, synthetic construct
ARVLTQTPSPVSAAVGDTVTINCQASQSVYGANQLSWYQQKPGQPPKLLIYQASKLASGVPSRLKGSGSGTQFTLTISEVQCDDAATYFCQGYYSGGIYTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 263 - protein, 444 aa, synthetic construct
QSLEESGGRLVTPGGSLTLTCTASGSDISPYTISWVRQAPEKGLEWIGIIGKSGGPYYATWAKGPFTISKTSTTVALKMTSLTAEDTATYFCGRYNGAGAMDIWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 264 - protein, 216 aa, synthetic construct
DPVLTQTPSSVFALVGGTVSINCQSSQSVYSMGCSWYQQKPGQPPKLLIYQASTLASGVSSRFKGSGSGTQFTLTISDVQCDDAATYYCLGGYSGNIYVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 265 - protein, 451 aa, synthetic construct
QSVEESGGRLVTPGTPLTLTCTISGFSLSSYDMSWVRQAPGEGLEWIGTISTGGSTYYASWAKGRFTISKTSSTTVDLKITSPTIEDTATYFCARDRGRSSYYTFGAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 266 - protein, 215 aa, synthetic construct
ALVMTQTPSSTSTAVGDTVTIKCQASQSITSYLSWFQQKPGQPPKLLIYLASTLASGVPSRFKGSGSGTEFTLTISGVQREDAATYYCLGSYSHSDTAFGGGTELELLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 267 - protein, 448 aa, synthetic construct
QLKESGGGLVQPGGSLKLSCKASGFDFSSYYMSWVRQAPGKGLEWIGWISSGGSTWYASWVNGRFTISSHNAQNTLYLQLNSLTAADTATYFCARDRDSSAWGDFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 268 - protein, 218 aa, synthetic construct
DPVMTQTPASVFEPVGGTVTINCQASESISNYLSWYQQKPGQPPKLLIYGASTLEFGVPSRFSGSGFGTEFSLTISGVQCDDAATYYCQGGYYSSSGTFFAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 444 aa, synthetic construct
QSXEESGGRLVTPGTPLTLTCTASGXSLSNYQMGWVRQAPGEGLEWIGIISSSGSAYYASWVNGRFTISKTSTTVDLKVTSLTTEDTATYFCARYLNNDYYDIWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 270 - protein, 217 aa, synthetic construct
AQVLTQTPSSVSAAVGGTVTINCQASQSISSYLAWYQQKRGQPPKLLIYYASNLAFGVSSRFKGSGFGTEYTLTISDLECDDAATYYCQQGYSDINVDNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 446 aa, synthetic construct
QSXEESGGXLVTPGTPLTLTCTVFGFSLSSNAMSWVRQAPGKGLEWIGFINRYGTTYYANWAKGRFTISKTSTTVDLKITSPTIEDTATYFCARADGVEYGYTRIWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 272 - protein, 217 aa, synthetic construct
ALVMTQTPSSVFAAVGGTVTINCQASQNIYSNLAWYQQKPGQRPNLLIYEASKMESGVPSRFSGSGSGPEYTLTISSVQCDDAATYYCQSGAYSASLDNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 273 - protein, 451 aa, synthetic construct
QSVEESGGXLVTPGTPLTLTCTIFGFSLSSYDMSWVRQAPGEGLEWIGTISTGGSTYYASWGKGRFTISKTSSTTVDLKITSPTMEDTATYFCARDRGRSSYYTFGAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 274 - protein, 215 aa, synthetic construct; identical to SEQ ID NO: 266

SEQ ID NO: 275 - protein, 443 aa, synthetic construct
QSVEESGGRLVTPGTPLTLTCTVSGFSLSRYGMSWVRQAPGKGLEWIGMISSSGSTYNPNWAKGRFTISKTSTTVDLKITSPTTEDTATYFCAREYWYYFNIWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 276 - protein, 219 aa, synthetic construct
AQVLTQTPSSVSAAVGGTVTINCQSSQSVYSTNYLAWYQQKPGQPPKLLIYDASSLASGVPSRFSGSGSGTQFTLTISGVQCDDAATYYCQGAYYASGWYHTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 277 - protein, 444 aa, synthetic construct
QSGEESGGRLVTPGTPLTLTCTAFGXSLSNYQMGWVRQAPGEGLEWIGIISSSGSAYYASWVNGRFTISKTSTTVDLKVTSLTTEDTATYFCARYLNNDYYDIWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - protein, 217 aa, synthetic construct
AQVLTQTPSSVSAAVGGTVTINCQASQSISSYLAWYQQKRGQPPKLLIYYASNLASGVSSRFKGSGFGTEYTLTISDLECDDAATYYCQQGYSDINVDNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 279 - protein, 10 aa, synthetic construct
GGGSSGGGSG

SEQ ID NO: 280 - protein, 12 aa, synthetic construct
ESKYGPPCPSCP

SEQ ID NO: 281 - protein, 12 aa, synthetic construct
ESKYGPPCPSCP

SEQ ID NO: 282 - protein, 22 aa, synthetic construct
ESKYGPPCPPCPGGGSSGGGSG

SEQ ID NO: 283 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 284 - protein, 228 aa, synthetic construct
ESKYGPPCPSCPAPEFEGGPSVFLFPPKPKDTLMISRTPEVTCVVVDVSQEDPEVQFNWYVDGVEVHQAKTKPREEQFSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 285 - protein, 229 aa, synthetic construct
ESKYGPPCPPCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 286 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 287 - protein, 21 aa, synthetic construct
LCYLLDGILFIYGVILTALFL

SEQ ID NO: 288 - protein, 22 aa, synthetic construct
MALIVLGGVAGLLLFIGLGIFF

SEQ ID NO: 289 - protein, 24 aa, synthetic construct
IYIWAPLAGTCGVLLLSLVITLYC

SEQ ID NO: 290 - protein, 27 aa, synthetic construct
IISFFLALTSTALLFLLFFLTLRFSVV

SEQ ID NO: 291 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 292 - protein, 41 aa, synthetic construct
RSKRSRLLHSDYMNMTPRRPGPTRKHYQPYAPPRDFAAYRS

SEQ ID NO: 293 - protein, 40 aa, synthetic construct
RSKRSRQHSDYMNMTPRRPGPTRKHYQPYAPPRDFAAYRS

SEQ ID NO: 294 - protein, 42 aa, synthetic construct
KRGRKKLLYIFKQPFMRPVQTTQEEDGCSCRFPEEEEGGCEL

SEQ ID NO: 295 - protein, 42 aa, synthetic construct
ALYLLRRDQRLPPDAHKPPGGGSFRTPIQEEQADAHSTLAKI

SEQ ID NO: 296 - protein, 214 aa, synthetic construct
DVQMTQSPSYLAASPGESVSIRCKASKSISNNLAWYQEKPGKANKLLIHSGSTLQSGTPSRFSGSGSGTDFTLTIRSLEFEDFAVYYCQQYNEYPYTFGAGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 297 - protein, 437 aa, synthetic construct
QVQLKESGPGLVQPSQTLSLTCTVSGFSLTTYSVHWVRQHSGKSLEWMGRMWTDGDTSYNSAFTSRLSISRDTSKSQVFLKMNSLQTEDTGTYYCARGYWYFDFWGPGTMVTVSSAETTAPSVYPLAPGTALKSNSMVTLGCLVKGYFPEPVTVTWNSGALSSGVHTFPAVLQSGLYTLTSSVTVPSSTWSSQAVTCNVAHPASSTKVDKKIVPRECNPCGCTGSEVSSVFIFPPKTKDVLTITLTPKVTCVVVDISQNDPEVRFSWFIDDVEVHTAQTHAPEKQSNSTLRSVSELPIVHRDWLNGKTFKCKVNSGAFPAPIEKSISKPEGTPRGPQVYTMAPPKEEMTQSQVSITCMVKGFYPPDIYTEWKMNGQPQENYKNTPPTMDTDGSYFLYSKLNVKKETWQQGNTFTCSVLHEGLHNHHTEKSLSHSPGK

SEQ ID NO: 298 - protein, 22 aa, synthetic construct
MLLLVTSLLLCELPHPAFLLIP

(19) United States
(12) Patent Application Publication
(10) Pub. No.: US 2026/0365746 A1
(43) Pub. Date: Nov. 19, 2026

(54) ANTI-LYMPHOTOXIN-B RECEPTOR (LTBR) ANTIBODIES AND METHODS OF USE THEREOF

(71) Applicant: CITY OF HOPE, Duarte, CA (US)

(72) Inventors: John WILLIAMS, Duarte, CA (US); Leo David WANG, Duarte, CA (US); Nour AMWAS, Duarte, CA (US); Miso PARK, Duarte, CA (US)

(73) Assignee: CITY OF HOPE, Duarte, CA (US)

(21) Appl. No.: 19/546,246

(22) Filed: Feb. 20, 2026

Related U.S. Application Data
(60) Provisional application No. 63/761,804, filed on Feb. 21, 2025.

(51) Int. Cl. C07K 16/28; A61K 35/17; A61K 39/00; A61K 40/11; A61K 40/31; A61K 40/42; A61K 45/06; A61P 35/00; C07K 14/54; C07K 14/705; C12N 5/0783

(52) U.S. Cl. CPC C07K 16/2878; A61K 35/17; A61K 40/11; A61K 40/31; A61K 40/4205; A61K 40/4211; A61K 45/06; A61P 35/00; C07K 14/5437; C07K 14/70517; C07K 14/70521; C12N 5/0636; A61K 2039/505; A61K 2239/13; A61K 2239/47; C07K 2317/24; C07K 2317/565; C07K 2317/622; C07K 2317/75; C12N 2510/00

(57) ABSTRACT

Provided herein are antibodies and single chain variable fragments (scFv) targeting LTβR. Also provided herein are populations of human T cells comprising an scFv targeting LTβR and methods of making the same. Also provided herein are methods of treating cancer with the antibodies, scFv, and populations of human T cells provided herein.

### CROSS-REFERENCES TO RELATED APPLICATIONS

This application claims the benefit of U.S. Provisional Patent Application No. 63/761,804, filed Feb. 21, 2025, which is incorporated herein in its entirety and for all purposes.

### REFERENCE TO A “SEQUENCE LISTING,” A TABLE, OR A COMPUTER PROGRAM LISTING APPENDIX SUBMITTED ON A COMPACT DISK

This application contains a Sequence Listing submitted electronically and is hereby incorporated by reference in its entirety. The Sequence Listing .xml file is entitled “CYH-006US1_Sequence_Listing_ST26.xml” is 282,307 bytes in size and was created on Feb. 20, 2026.

### BACKGROUND

Clinical outcomes in aggressive brain tumors have scarcely improved in the past 20 years. Although significant enthusiasm exists for the application of immunotherapies such as chimeric antigen receptor T (CAR-T) cell therapy, initial results have been disappointing, in part because brain tumors are immunosuppressive or “cold”. “Cold” tumors are tumors that are surrounded by cells that suppress immune responses and prevent T cells from attacking the tumor. As such, “cold” tumors do not typically respond well to immunotherapies. Thus, the immune suppressive milieu of brain tumors hinders an endogenous immune response by preventing dendritic cell maturation, promoting abnormal angiogenesis, and enabling antigen escape, and also drives hypofunction and exhaustion of CAR-T cells. In contrast, some tumors are considered “hot” tumors, which typically respond better to immunotherapies and have higher T cell infiltration. One strategy for improving CAR T efficacy in “cold” tumors is to target the tumor microenvironment (TME) and convert it from a “cold” to a “hot” tumor. However, there is a need for developing such strategies.

### SUMMARY

Provided herein is a lymphotoxin-β receptor (LTβR) antibody comprising a light chain variable region and a heavy chain variable region. Optionally, the antibody is a single chain variable fragment (scFv), a humanized antibody, or a chimeric antibody. In another aspect, a LTβR antibody comprising a heavy chain and a light chain is provided.

A scFv that binds LTβR comprising a light chain variable region and a heavy chain variable region is also provided. The scFv can further include a hinge domain, e.g., a CD28 hinge domain or a CD8 hinge domain. The scFv can also include a transmembrane domain, e.g., a CD28 transmembrane domain or a CD8 transmembrane domain. Optionally, the scFv further comprises a linker sequence.

Also provided herein is a nucleic acid molecule encoding the LTβR antibody or scFv that binds LTβR. A vector comprising the nucleic acid molecule encoding the LTβR antibody or scFv that binds LTβR is also provided. A pharmaceutical composition comprising the LTβR antibody or scFv that binds LTβR and a pharmaceutically acceptable excipient is also provided.

Methods of treating cancer in a subject in need thereof comprising administering to a subject a therapeutically effective amount of the pharmaceutical composition comprising the LTβR antibody or scFv that binds LTβR and a pharmaceutically acceptable excipient is provided. The cancer can be a brain cancer. The methods can also include administering to the subject a second therapeutic agent, which can be, for example, an immune checkpoint inhibitor.

Also provided is a population of human T cells comprising the scFv that binds LTβR. Optionally, the population of human T cells further comprises a chimeric antigen receptor (CAR). The CAR can include a HER2 targeting sequence, a CD19 targeting sequence or an Interleukin Receptor targeting sequence. Optionally, the CAR comprises an interleukin, e.g., interleukin 13 (IL13).

Methods of treating cancer in a subject in need thereof comprising the population of human T cells is also provided. Optionally, the cancer is brain cancer.

Methods of making a population of T cells expressing a membrane bound scFv targeted to LTβR are also provided. The methods include obtaining the population of T cells from a subject, transducing the population of T cells with a nucleic acid sequence encoding the scFv, and isolating the population of T cells expressing the membrane bound scFv targeted to the LTβR. The methods of making the population of T cells can also include modifying the population of T cells to express a CAR. The CAR can comprise a HER2 targeting sequence, a CD19 targeting sequence or an Interleukin Receptor targeting sequence. Optionally, the CAR comprises an interleukin, e.g., IL13.

### BRIEF DESCRIPTION OF THE DRAWINGS

FIG. 1A-B test cross reactivity of commercially available anti-LTβR antibodies and agonistic activity. FIG. 1A is an image of a western blot assessment of noncanonical NF-κB signaling (p100 processing to p52) of NIH3T3 cells (a murine cell line) and HT1080 cells (a human cell line) treated with antibody. FIG. 1B Screening monoclonal anti-LTβR antibodies in NIH3T3 NF-κB reporter cells.

FIG. 2 is an exemplary illustration of an experimental overview to assess tertiary lymphoid structure (TLS) formation in mice treated with 5G11 antibody.

FIG. 3A are images of Hematoxylin and Eosin (H&E) staining of TLS in mice treated with 5G11 antibody versus mice treated with PBS (control). FIG. 3B is a graph showing the cell number/cluster (top) and total number of clusters/tumor area in mm² (bottom) in mice treated with 5G11 antibody versus mice treated with PBS (control), showing that treatment with 5G11 anti-mLTβR antibody in KR158 glioma-bearing mice significantly increased the number of lymphocyte clusters per tumor area and the average cell number per cluster compared to controls, indicating enhanced lymphocyte aggregation and infiltration.

FIG. 4A are opal immunofluorescence immunohistochemistry tumor section images showing B cell, T cell, and dendritic cell infiltration (B220+, CD3+, and CD11c+ cells) into tumor in mice treated with 5G11 antibody versus mice treated with PBS (control). FIG. 4B are graphs quantifying B cell, T cell, and dendritic cells per tumor area in mm² in mice treated with 5G11 antibody versus mice treated with PBS (control).

FIG. 5 shows an exemplary construct design and T cell membrane placement for (i) membrane-tethered agonist (mTa) containing heavy and light chain variable domains of an scFv binding to LTβR. mIL13CAR construct was used to evaluate the function of mTa in improving response to CAR T. mTa and IL13-CAR were on separate vectors and T cells are co-transduced on the same day to produce mTa-IL13-CAR T.

FIG. 6 are flow cytometry plots showing single and dual transduction of mTa T cells and/or IL13-CAR T cells in mTa-IL13-CAR T cells compared to mock T cells, and fluorescence minus one (FMO) GS43 and CD19 controls. Anti G4S3 and CD19-conjugated antibodies were used for the detection of mTa and CAR expression, respectively.

FIG. 7A-B shows activity of mTa alone or mTa-CART to activate NFkB pathway in reporter cell lines and WB. FIG. 7A are immunofluorescent microscopy images showing NFkB signaling in a NIH3T3 NFkB-GFP reporter cell line treated with either 5 μM of birinapant or 100 ng of 5G11, or co-cultured with mock T cells, co-cultured with mTa T cells, or co-cultured with mTa-IL13CAR T cells at a 1:1 ratio. Functional activation of LTβR was validated by coculturing mTa-expressing T cells with NIH3T3 NF-κB-GFP reporter cells. FIG. 7B shows a western blot analysis of cell lysates that demonstrates the processing of p100 to p52, confirming downstream LTβR signaling.

FIG. 8A is a graph showing LTβR expression on mTa-mIL13 CAR T cells, IL13 CAR T cells, mTa T cells, mock T cell, IL13Ra2-KLuc KR158 cells, and wildtype KLuc-KR158 cells. FIG. 8B are flow cytometry plots showing single and dual transduction efficiency of mTa T cells, IL13-CAR-T cells, and mTa-IL13-CAR T cells compared to non-transduced mock T cells. G4S3 and CD19-conjugated antibodies were used for the detection of mTa and CAR expression, respectively. FIG. 8C is a graph showing the expression of mTa on IL13-CAR T does not affect CAR mediated killing in vitro.

FIG. 9 is an exemplary illustration of an experimental overview to assess performance of mTa T cells in vivo in mice injected with KR158-luc cells.

FIGS. 10A-C are images showing tumor volume in mice treated with mTa T cells, mock T cells, 5G11 antibody, or PBS (control) at days 8 (FIG. 10A), 12 (FIG. 10B), and 18 (FIG. 10C) post KR158-luc cell injection.

FIG. 11 is a graph showing tumor volume in regions of interest from FIGS. 10A-C post KR158-luc cell injection.

FIGS. 12A-G show that mTa expression does not impair CAR-T therapeutic efficacy in vivo. FIGS. 12A-F show bioluminescence imaging-derived measurements of tumor burden across different treatment groups: 5G11 (FIG. 12A), mock T (FIG. 12B), mTa (FIG. 12C), mTa-CART (FIG. 12D), CART (FIG. 12E), and tumor burden (FIG. 12F). FIG. 12G shows the in vivo survival rates corresponding to each group.

FIGS. 13A-E show that repeated LTβR agonist administration is well tolerated and associated with reduced tumor growth, improved survival, and increased immune cell infiltration. FIG. 13A is a graph showing mice body weight over time stratified by treatment group and sex. Bold lines represent the median weight at each time point. FIG. 13B is a graph showing individual tumor growth curves for each mouse. Bold lines represent the median tumor volume per treatment group at each time point. FIG. 13C is a graph showing a Kaplan-Meier survival analysis comparing 5G11-treated and PBS-treated mice. FIG. 13D is a graph showing absolute counts of tumor-infiltrating immune cells at endpoint, normalized to tumor weight (cells per mg tumor tissue), as determined by flow cytometry. FIG. 13E shows the H&E slides with annotated TLS and lymphoid aggregates from sectioned tumors. FIG. 13F the number of TLS and aggregates per tumor was quantified and graphed according to treatment group.

FIGS. 14A-B illustrate flow cytometry analysis assessing binding of human antibodies to LTβR expressing cells. FIG. 14A depicts flow cytometry histograms showing detection of bound human antibodies using an anti-human (H+L) secondary antibody in HT1080 (LTβR⁺) and Jurkay (LTβR⁻) cells. FIG. 14B shows flow cytometry dot plots of HT1080 (LTβR⁺) and Jurkay (LTβR⁻) cells incubated with LTβR antibodies.

FIG. 15 shows the functional activity of human LTβR antibodies.

FIGS. 16A-B provide a summary of the experimental results evaluating the effect of candidate antibody treatments on LTβR-mediated non-canonical NF-κB signaling in HT1080 cells. FIG. 16A shows a western blot analysis depicting the processing of the NF-κB precursor protein P100 to its active form, P52, following treatment with either soluble antibodies or antibodies immobilized on beads. FIG. 16B shows a graph of P52/actin band intensity for soluble antibodies and immobilized antibodies on beads.

FIG. 17 shows an exemplary construct design of two human LTβR candidates converted into membrane-tethered scFv constructs for translational evaluation.

FIGS. 18A-G illustrate the production, screening and characterization of mouse LTβR (mLTβR) and human LTβR (lLTβR). FIG. 18A shows a multiple sequence alignment comparing amino acid sequences of mLTβR and hLTβR. FIG. 18B shows size-exclusion chromatography (SEC) profiles of mLTβR and hLTβR. FIG. 18C illustrates sensorgram plots showing binding of hLTβR to commercial anti-hLTβR antibody at multiple antibody concentrations as a function of time. FIG. 18D images showing positive detection of mLTβR and hLTβR reactive antibodies from memory B cells isolated from immunized rabbits. FIG. 18E shows western blot demonstrating successful production of nine hLTβR Fab fragments under reducing and non-reducing conditions and FIG. 18F depicts their respective Surface Plasmon Resonance (SPR) graphs showing binding affinity and specificity. FIG. 18G shows mLTβR-hLTβR (MH1) hybrid binding on SPR to assess species cross-reactivity and specificity of the antibodies. FIG. 18H graph shows the protein thermal shift for LTβR 2S6 Fab, LTβR 2S48 Fab, and LTβR 2S49 Fab.

### DETAILED DESCRIPTION

### Definitions

While various some aspects and aspects of the present invention are shown and described herein, such some aspects and aspects are provided by way of example only. Numerous variations, changes, and substitutions will now occur to those skilled in the art without departing from the invention. It should be understood that various alternatives to the some aspects described herein may be employed.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 3rd ed., J. Wiley & Sons (New York, NY 2006); Sambrook et al., MOLECULAR CLONING, A LABORATORY MANUAL, 4th ed., Cold Springs Harbor Press (Cold Springs Harbor, NY 2014). Any methods, devices and materials similar or equivalent to those described herein can be used. The following definitions are provided to facilitate understanding of certain terms used frequently herein and are not meant to limit the scope of the present disclosure.

“Nucleic acid” refers to deoxyribonucleotides or ribonucleotides and polymers thereof in either single- or double-stranded form and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents but retain the same basic chemical structure as the naturally occurring nucleic acid. A nucleic acid mimetic refers to chemical compounds that have a structure that is different the general chemical structure of a nucleic acid, but that functions in a manner like a naturally occurring nucleic acid. Examples of such analogues include, without limitation, phosphorothioates, phosphoramidates, methyl phosphonates, chiral-methyl phosphonates, 2-O-methyl ribonucleotides, and peptide-nucleic acids (PNAs).

The term “amino acid” refers to naturally occurring and synthetic amino acids, as well as amino acid analogs and amino acid mimetics that function in a manner like the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to”, when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. For example, a selected residue in a selected antibody (or Fab domain) corresponds to light chain threonine at Kabat position 40, when the selected residue occupies the same essential spatial or other structural relationship as a light chain threonine at Kabat position 40. In some aspects, where a selected protein is aligned for maximum homology with the light chain of an antibody (or Fab domain), the position in the aligned selected protein aligning with threonine 40 is said to correspond to threonine 40. Instead of a primary sequence alignment, a three-dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations.

Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
   (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, In some aspects 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. In some aspects, the identity exists over a region that is at least about 50 nucleotides in length, or more preferably over a region that is 100 to 500 or 1000 or more nucleotides in length.

Percentage of sequence identity is determined by comparing two optimally aligned sequences over a comparison window, wherein the portion of the polynucleotide or polypeptide sequence in the comparison window may comprise additions or deletions (z.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2: 482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) AMC. Acids Res. 25:3389-3402, and Altschul et al. (1990) Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Set. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Set. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “antibody” is used according to its commonly known meaning in the art. An antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments called the complementarity determining regions (“CDRs”). The six CDRs in an antibody variable domain (three from the light chain and three from the heavy chain) fold up together in 3-dimensional space to form the actual antibody binding site which binds the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific F(ab)2, bispecific F(ab)2, trispecific F(ab)2, monovalent IgGs, single chain variable fragments (scFv), membrane tethered agonist (mTa), bispecific antibodies, bispecific diabodies, trispecific triabodies, scFv-Fc, minibodies, IgNAR, V-NAR, hdgG, VhH, or peptibodies. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. The variable light chains provided herein can include in N-terminal to C-terminal direction a CDR LI, a CDR L2 and a CDR L3. Likewise, the terms “CDR HI”, “CDR H2” and “CDR H3” as provided herein refer to the complementarity determining regions (CDR) 1, 2, and 3 of the variable heavy (H) chain of an antibody. The variable heavy chains provided herein can include in N-terminal to C-terminal direction a CDR H1, a CDR H2 and a CDR H3.

The terms “FR LI”, “FR L2”, “FR L3” and “FR L4” as provided herein are used according to their common meaning in the art and refer to the framework regions (FR) 1, 2, 3 and 4 of the variable light (L) chain of an antibody. In some aspects, the variable light chain provided herein includes in N-terminal to C-terminal direction a FR LI, a FR L2, a FR L3 and a FR L4. Likewise, the terms “FR Hl”, “FR H2”, “FR H3” and “FR H4” as provided herein are used according to their common meaning in the art and refer to the framework regions (FR) 1, 2, 3 and 4 of the variable heavy (H) chain of an antibody. In some aspects, the variable heavy chain provided herein includes in N-terminal to C-terminal direction a FR Hl, a FR H2, a FR H3 and a FR H4.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa. An scFv may be provided as a membrane-bound or membrane-tethered version comprising a hinge and/or transmembrane domain. For example, a membrane-tethered agonist (mTa) is a membrane bound scFv agonist (i.e., an scFv which binds to and induces activity of a target).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, and the like; or (b) the variable region, or a portion thereof, is altered, replaced or exchanged with a variable region having a different or altered antigen specificity.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31 (3): 169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologies. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solidphase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “lymphotoxin beta receptor”, “LTβR” or “LTβR protein” as referred to herein includes any of the recombinant or naturally-occurring forms of the lymphotoxin beta receptor (LTβR) also known as tumor necrosis factor receptor superfamily member 3 (TNFRSF3 or TNR3) or variants or homologs thereof that maintain LTβR activity (e.g. within at least 50%, 80%, 90%, 95%, 96%, 97%, 98%, 99% or 100% activity compared to LTβR. In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring LTβR. In some aspects, the LTβR is substantially identical to the protein identified by the UniProt reference number P36941 or a variant or homolog having substantial identity thereto. In some aspects, the LTβR is substantially identical to the protein identified by the UniProt reference number F5H627 or a variant or homolog having substantial identity thereto. In some aspects, the LTβR is substantially identical to the protein identified by the NCBI reference number GI:4055 or a variant or homolog having substantial identity thereto. In some aspects, the LTβR comprises SEQ ID NO: 1 or an amino acid sequence having 90%, 95%, 96%, 97%, 98%, 99% or 100% sequence identity to SEQ ID NO: 1. In some aspects, the LTβR comprises SEQ ID NO: 2 or an amino acid sequence having 90%, 95%, 96%, 97%, 98%, 99% or 100% sequence identity to SEQ ID NO: 2.

The terms “agonist,” “activator,” “upregulator,” etc. refer to a substance capable of detectably increasing the expression or activity of a given gene or protein (e.g., LTβR). The agonist can increase expression or activity 10%, 20%, 30%, 40%, 50%, 60%, 70%, 80%, 90% or more in comparison to a control in the absence of the agonist. In some aspects, agonist can increase expression or activity 10% in comparison to a control in the absence of the agonist. In some aspects, agonist can increase expression or activity 20% in comparison to a control in the absence of the agonist. In some aspects, agonist can increase expression or activity 30% in comparison to a control in the absence of the agonist. In some aspects, agonist can increase expression or activity 40% in comparison to a control in the absence of the agonist. In some aspects, agonist can increase expression or activity 50% in comparison to a control in the absence of the agonist. In some aspects, agonist can increase expression or activity 60% in comparison to a control in the absence of the agonist. In some aspects, agonist can increase expression or activity 70% in comparison to a control in the absence of the agonist. In some aspects, agonist can increase expression or activity 80% in comparison to a control in the absence of the agonist. In some aspects, agonist can increase expression or activity 90% in comparison to a control in the absence of the agonist. In some aspects, agonist can increase expression or activity 95% in comparison to a control in the absence of the agonist. In some aspects, agonist can increase expression or activity 95% or more in comparison to a control in the absence of the agonist. In certain instances, expression or activity is 1.5-fold, 2-fold, 3-fold, 4-fold, 5-fold, 10-fold or higher than the expression or activity in the absence of the agonist. In some aspects, expression or activity is 1.5-fold higher than the expression or activity in the absence of the agonist. In some aspects, expression or activity is 2-fold higher than the expression or activity in the absence of the agonist. In some aspects, expression or activity is 3-fold higher than the expression or activity in the absence of the agonist. In some aspects, expression or activity is 4-fold higher than the expression or activity in the absence of the agonist. In some aspects, expression or activity is 5-fold higher than the expression or activity in the absence of the agonist. In some aspects, expression or activity is 10-fold higher than the expression or activity in the absence of the agonist. In some aspects, expression or activity is 10-fold or higher than the expression or activity in the absence of the agonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

A “lymphoid tissue inducer cell” as referred to herein is a cell characterized by the ability to contribute to the formation of lymphoid structures (e.g., lymph nodes and tertiary lymphoid structures). In the context of tumor associated-tertiary lymphoid structures, the lymphoid tissue inducer cells are a hematopoietic cell type and generally express that express LTα1β2 that binds to the LTβR on lymphoid tissue organizer cells. RORγt and IL-7Ra in the absence of lineage markers (e.g. CD3, CD19, B220, CD11c, Gr-1).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, synoviocytes, synovial fluid, synovial tissue, fibroblast-like synoviocytes, macrophagelike synoviocytes, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some aspects, a patient is human.

The terms “disease” or “condition” refer to a state of being or health status of a patient or subject capable of being treated with the compounds or methods provided herein. The disease may be a cancer. As used herein, the term “cancer” refers to all types of cancer, neoplasm or malignant tumors found in mammals (e.g., humans), including leukemia, carcinomas and sarcomas. Exemplary cancers that may be treated with a compound or method provided herein include breast cancer, colon cancer, kidney cancer, leukemia, lung cancer, melanoma, ovarian cancer, prostate cancer, pancreatic cancer, brain cancer, liver cancer, gastric cancer or a sarcoma. Optionally, the cancer is a brain cancer. Optionally, the cancer is a glioblastoma. Optionally, the cancer is non-small cell lung cancer, breast cancer, a melanoma, colorectal cancer, pancreatic cancer or ovarian cancer or any other cancer associated with TLS.

As used herein, “treating” or “treatment of” a condition, disease or disorder or symptoms associated with a condition (e g., cancer), disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus, in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

A “therapeutic agent” as referred to herein, is a composition useful in treating or preventing a disease such as cancer (e.g., glioblastoma). The therapeutic agent can be an anti-cancer agent. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In some aspects, an anti-cancer agent is a chemotherapeutic agent. In some aspects, an anti-cancer agent is an agent identified herein having utility in methods of treating cancer. In some aspects, an anti-cancer agent is an agent approved by the FDA or similar regulatory agency of a country other than the USA, for treating cancer.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds can be administered alone or can be coadministered to a subject. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In some aspects, the formulations of the compositions of the present invention can be delivered using liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Cnrr. Opin. Biotechnol. 6:698-708, 1995; Ostro, Am. J. EIosp. Pharm. 46:1576-1587, 1989). The compositions of the present invention can also be delivered as nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

### Antibodies

The presence of tertiary lymphoid structures (TLS) is positively associated with improved overall survival rate and response to immunotherapy correlated with the increased memory T and B cells, tumor reactive infiltrating T cells, production of tumor reactive antibodies, and maturation of dendritic cells. For example, high TLS expression is associated with longer overall survival and relapse-free survival in solid tumors, as well as a reduced risk of cancer relapse. High TLS expression is also associated with smaller tumor size, increased infiltration of TILs, lower clinical grade, and reduced N stage in tumor (Zhao et al., “Relationship between Tertiary Lymphoid Structure and the Prognosis and Clinicopathologic Characteristics in Solid Tumors.” Int J Med Sci. 2021; 18 (11): 2327-2338. Published 2021 Apr. 7). Certain TLSs exhibit features of dynamic immune functions, including clonal T and B cell expansion, generation of IgA+ and IgG+ plasma cells, and dendritic cell-T cell interactions. The presence of TLSs with active immune response features correlates with improved overall survivor (Cakmark et al., “Spatial immune profiling defines a subset of human gliomas with functional tertiary lymphoid structures.” Immunity, vol. 58, 11 (2025): 2847-2863.e8; Bruno, “New Predictors for immunotherapy responses sharpen our view of the tumour microenvironment.” Nature, 577 (7791): 474-476; January 2020). Moreover, mature TLSs can predict immune checkpoint inhibitor efficacy in solid tumors independently of PD-L1 expression. Thus, TLS detection can be used to select patients who are more likely to benefit from immune checkpoint blockade (Vanhersecke et al., “Mature tertiary lymphoid structures predict immune checkpoint inhibitor efficacy in solid tumors independently of PD-L1 expression.” Nature Cancer, 2, 794-802; 2021). Therefore, identifying strategies to augment TLS in GBM can potentially improve survival and response to CAR-T therapy. Provided herein are, inter alia, antibodies and antibody variants (e.g., humanized antibodies, scFvs, or mTas) capable of binding lymphotoxin beta receptor (LTβR). The antibodies provided herein include novel light chain and heavy chain sequences and bind LTβR with high efficiency and specificity, thereby effectively targeting LTβR expressing cells. The antibodies and antibody variants that bind LTβR provided herein can induce nuclear factor kappa-B (NF-kB) signaling to induce the expression of genes involved in lympho-neogenesis and formation of tertiary lymphoid structures. LTβR signaling is a critical regulator of lymph node organogenesis and can cooperate with antiangiogenic and immune checkpoint blockade treatment to augment tumor-associated HEV formation. Systemic activation of the LTβR pathway via agonistic antibody treatment induces tumor-specific HEV formation, upregulated the expression of TLS-related chemokines, and enhanced dendritic cell (DC) and T-cell infiltration and activation in syngeneic tumor models. In vitro studies confirmed direct effects of LTβR agonism on DC activation and maturation and associated DC-mediated T-cell activation. Single-agent LTβR agonist treatment inhibited syngeneic tumor growth in a CD8+ T-cell-dependent and HEV-dependent manner, and the LTβR agonist enhanced antitumor effects of anti-PD-1 and CAR T-cell therapies. An in vivo tumor screen for TLS-inducing cytokines revealed that the combination of LTβR agonism and lymphotoxin a expression promoted robust intratumoral TLS induction and enhanced tumor responses to anti-CTLA4 treatment (Disi An et al., “LTβR Agonism Promotes Antitumor Immune Response via Modulation of the Tumor Microenvironment.” Cancer Res., 84 (23): 3984-4001; Dec. 2, 2024). Tertiary lymphoid structures (TLS) are ectopic lymphoid aggregates that arise in response to chronic inflammation and mimic the structure and function of secondary lymphoid organs. The spontaneous presence of TLS in some solid tumors, including GBM, is associated with improved clinical outcome and responsiveness to immunotherapy. Thus, the anti-LTβR antibodies and antibody variants provided herein are, inter alia, useful for the improving clinical outcome and responsiveness to therapeutic treatments for cancer.

### Antibodies Binding Human LTβR

Provided herein are lymphotoxin beta receptor (LTβR) antibody or antibody variants which bind to human LTβR (SEQ ID NO: 2). In one aspect, a LTβR antibody or antibody variant including a light chain variable region and a heavy chain variable region is provided. The light chain variable region includes a CDR L1, a CDR L2, and a CDR L3 and the heavy chain variable region includes a CDR H1, a CDR H2, and a CDR H3. In some aspects, the CDR L1, CDR L2, and CDR L3 have amino acid sequences as set forth in SEQ ID NOs: 5, 6, and 7 and the CDR H1, CDR H2, and CDR H3 have amino acid sequences as set forth in SEQ ID NOs: 8, 9 and 10. In some aspects, the CDR L1, CDR L2, and CDR L3 have amino acid sequences as set forth in SEQ ID NOs: 13, 14 and 15 and the CDR H1, CDR H2, and CDR H3 have amino acid sequences as set forth in SEQ ID NOs: 16, 17 and 18. In some aspects, the CDR L1, CDR L2, and CDR L3 have amino acid sequences as set forth in SEQ ID NOs: 21, 22, and 23 and the CDR H1, CDR H2, and CDR H3 have amino acid sequences as set forth in SEQ ID NOs: 24, 25 and 26. In some aspects, the CDR L1, CDR L2, and CDR L3 have amino acid sequences as set forth in SEQ ID NOs: 29, 30, and 31 and the CDR H1, CDR H2, and CDR H3 have amino acid sequences as set forth in SEQ ID NOs: 32, 33 and 34. In some aspects, the CDR L1, CDR L2, and CDR L3 have amino acid sequences as set forth in SEQ ID NOs: 37, 38 and 39 and the CDR H1, CDR H2, and CDR H3 have amino acid sequences as set forth in SEQ ID NOs: 40, 41, and 42. In some aspects, the CDR L1, CDR L2, and CDR L3 have amino acid sequences as set forth in SEQ ID NOs: 45, 46 and 47 and the CDR H1, CDR H2, and CDR H3 have amino acid sequences as set forth in SEQ ID NOs: 48, 49 and 50. In some aspects, the CDR LI, CDR L2, and CDR L3 have amino acid sequences as set forth in SEQ ID NOs: 53, 54 and 55 and the CDR H1, CDR H2, and CDR H3 have amino acid sequences as set forth in SEQ ID NOs: 56, 57 and 58. In some aspects, the CDR L1, CDR L2, and CDR L3 have amino acid sequences as set forth in SEQ ID NOs: 61, 62 and 63 and the CDR H1, CDR H2, and CDR H3 have amino acid sequences as set forth in SEQ ID NOs: 64, 65 and 66. In some aspects, the CDR L1, CDR L2, and CDR L3 have amino acid sequences as set forth in SEQ ID NOs: 69, 70 and 71 and the CDR H1, CDR H2, and CDR H3 have amino acid sequences as set forth in SEQ ID NOs: 72, 73 and 74. In some aspects, the CDR L1, CDR L2, and CDR L3 have amino acid sequences as set forth in SEQ ID NOs: 77, 78 and 79 and the CDR H1, CDR H2, and CDR H3 have amino acid sequences as set forth in SEQ ID NOs: 80, 81 and 82. In some aspects, the CDR L1, CDR L2, and CDR L3 have amino acid sequences as set forth in SEQ ID NOs: 85, 86, and 87 and the CDR H1, CDR H2, and CDR H3 have amino acid sequences as set forth in SEQ ID NOs: 88, 89 and 90. In some aspects, the CDR L1, CDR L2, and CDR L3 have amino acid sequences as set forth in SEQ ID NOs: 53, 93 and 94 and the CDR H1, CDR H2, and CDR H3 have amino acid sequences as set forth in SEQ ID NOs: 95, 57 and 58. In some aspects, the CDR L1, CDR L2, and CDR L3 have amino acid sequences as set forth in SEQ ID NOs: 98, 99 and 100 and the CDR H1, CDR H2, and CDR H3 have amino acid sequences as set forth in SEQ ID NOs: 101, 102 and 103. In some aspects, the CDR L1, CDR L2, and CDR L3 have amino acid sequences as set forth in SEQ ID NOs: 108, 109 and 110 and the CDR H1, CDR H2, and CDR H3 have amino acid sequences as set forth in SEQ ID NOs: 111, 112, and 113. In some aspects, the CDR LI, CDR L2, and CDR L3 have amino acid sequences as set forth in SEQ ID NOs: 116, 117 and 118 and the CDR H1, CDR H2, and CDR H3 have amino acid sequences as set forth in SEQ ID NOs: 119, 120 and 121.

In some aspects, the light chain variable region comprises SEQ ID NO:3 and the heavy chain variable region comprises SEQ ID NO:4. In some aspects, the light chain variable region comprises SEQ ID NO:11 and the heavy chain variable region comprises SEQ ID NO: 12. In some aspects, the light chain variable region comprises SEQ ID NO: 19 and the heavy chain variable region comprises SEQ ID NO:20. In some aspects, the light chain variable region comprises SEQ ID NO:27 and the heavy chain variable region comprises SEQ ID NO:28. In some aspects, the light chain variable region comprises SEQ ID NO:35 and the heavy chain variable region comprises SEQ ID NO:36. In some aspects, the light chain variable region comprises SEQ ID NO:43 and the heavy chain variable region comprises SEQ ID NO:44. In some aspects, the light chain variable region comprises SEQ ID NO:51 and the heavy chain variable region comprises SEQ ID NO:52. In some aspects, the light chain variable region comprises SEQ ID NO:59 and the heavy chain variable region comprises SEQ ID NO:60. In some aspects, the light chain variable region comprises SEQ ID NO:67 and the heavy chain variable region comprises SEQ ID NO:68. In some aspects, the light chain variable region comprises SEQ ID NO:75 and the heavy chain variable region comprises SEQ ID NO:76. In some aspects, the light chain variable region comprises SEQ ID NO:83 and the heavy chain variable region comprises SEQ ID NO:84. In some aspects, the light chain variable region comprises SEQ ID NO:91 and the heavy chain variable region comprises SEQ ID NO:92. In some aspects, the light chain variable region comprises SEQ ID NO:96 and the heavy chain variable region comprises SEQ ID NO:97. In some aspects, the light chain variable region comprises SEQ ID NO: 104 and the heavy chain variable region comprises SEQ ID NO: 105. In some aspects, the light chain variable region comprises SEQ ID NO:106 and the heavy chain variable region comprises SEQ ID NO:107. In some aspects, the light chain variable region comprises SEQ ID NO:114 and the heavy chain variable region comprises SEQ ID NO:115.

In some aspects, the LTβR antibody or antibody variant is a humanized antibody. A humanized antibody is a genetically engineered antibody in which at least one CDR (or functional fragment thereof) from a non-human antibody (“donor antibody”, which can be mouse, rabbit, rat, hamster or other non-human species) are grafted onto a human antibody (“acceptor antibody”). In some aspects, more than one donor CDR is grafted (e.g. all six donor CDRs are grafted). The sequence of the acceptor antibody can be, for example, a mature human antibody sequence (or fragment thereof), a consensus sequence of a human antibody sequence (or fragment thereof), or a germline region sequence (or fragment thereof). Thus, a humanized antibody may be an antibody having one or more CDRs from a donor antibody and a variable region framework (FR). The FR may form part of a constant region and/or a variable region within a human antibody. In addition, in order to retain high binding affinity, amino acids in the human acceptor sequence may be replaced by the corresponding amino acids from the donor sequence, for example where: (1) the amino acid is in a CDR or (2) the amino acid is in the human framework region (e.g. the amino acid is immediately adjacent to one of the CDRs). See, U.S. Pat. Nos. 5,530,101 and 5,585,089, incorporated herein by reference, which provide detailed instructions for construction of humanized antibodies. Although humanized antibodies often incorporate all six CDRs (e.g. as defined by Kabat, but often also including hypervariable loop H1 as defined by Chothia) from a donor antibody, they can also be made with fewer donor CDRs and/or less than the complete donor CDR sequence (e.g. a functional fragment of a CDR) (e.g., Pascalis et al., J. Immunol. 169:3076, 2002; Vajdos et al., Journal of Molecular Biology, 320:415-428, 2002; Iwahashi et al., Mol. Immunol. 36:1079-1091, 1999; Tamura et al, Journal of Immunology, 164:1432-1441, 2000).

Typically, a humanized antibody as provided herein may include (i) a light chain variable region comprising at least one CDR (often three CDRs) from a donor antibody and a human variable region framework; and (ii) a heavy chain variable region comprising at least one CDR (often three CDRs) from the donor antibody and a human variable region framework (FR). The light and heavy chain variable region frameworks (FRs) may each be a mature human antibody variable region framework sequence (or fragment thereof), a germline variable region framework sequence (combined with a J region sequence) (or fragment thereof), or a consensus sequence of a human antibody variable region framework sequence (or fragment thereof). In some aspects, the humanized antibody includes a light chain variable region as described in (i), a heavy chain variable region as described in (ii) together with a light chain human constant region and a heavy chain human constant region.

Where the LTβR antibody or antibody variant provided herein is a humanized antibody, the antibody may include a humanized heavy chain variable region and/or a humanized light chain variable region. In some aspects, the humanized light chain variable region and the humanized heavy chain variable region include combined one or more donor (e.g. mouse, rat, rabbit) CDRs or functional fragments of a donor CDR. Thus, in some aspects, the humanized light chain variable region and the humanized heavy chain variable region include combined six CDRs wherein at least one (e.g., one, two, three, four, five, or all six) of the six CDRs is a donor CDR. For example, a humanized antibody may include CDR L3 derived from the donor antibody and CDR L1, CDR L2, CDR H1, CDR H2, and CDR H3 derived from the acceptor antibody (i.e. human).

Optionally, the LTβR antibody or antibody variant is a chimeric antibody. Optionally, the chimeric antibody is an antibody in which the variable region of a donor (e.g., mouse, rabbit, rat, hamster or other non-human species) antibody is combined with the constant region of a human antibody; the construction of a chimeric antibody by means of genetic engineering is well-known. Such antibodies retain the binding specificity of the donor antibody, while being about two-thirds human. The proportion of nonhuman sequence present in donor, chimeric and humanized antibodies suggests that the immunogenicity of chimeric antibodies is intermediate between donor and humanized antibodies. Other types of genetically engineered antibodies that may have reduced immunogenicity relative to donor antibodies include human antibodies made using phage display methods (Dower et al., WO91/17271; McCafferty et al., WO92/001047; Winter, WO92/20791; and Winter, FEBS Lett. 23:92, 1998, each of which is incorporated herein by reference) or using transgenic animals (Lonberg et al., WO93/12227; Kucherlapati WO91/10741, each of which is incorporated herein by reference).

Other approaches to design humanized antibodies may also be used to achieve the same result as the methods in U.S. Pat. Nos. 5,530,101 and 5,585,089 described above, for example, “superhumanization” (see Tan et al. J. Immunol. 169:1119, 2002, and U.S. Pat. No. 6,881,557) or the method of Studnicak et al., Protein Eng. 7:805, 1994. Moreover, other approaches to produce genetically engineered, reduced-immunogenicity mAbs include “reshaping”, “hyperchimerization” and veneering/resurfacing, as described, e.g., in Vaswami et al., Annals of Allergy, Asthma and Immunology 81:105, 1998; Roguska et al. Protein Eng. 9:895, 1996; and U.S. Pat. Nos. 6,072,035 and 5,639,641.

In another aspect, a chimeric LTβR antibody including a heavy chain and a light chain is provided. In some aspects, the heavy chain comprises an amino acid sequence set forth in SEQ ID NO: 122 and the light chain comprises an animo acid sequence set forth in SEQ ID NO: 123. In some aspects, the heavy chain comprises an amino acid sequence set forth in SEQ ID NO:124 and the light chain comprises an animo acid sequence set forth in SEQ ID NO:125. In some aspects, the heavy chain comprises an amino acid sequence set forth in SEQ ID NO: 126 and the light chain comprises an animo acid sequence set forth in SEQ ID NO: 127. In some aspects, the heavy chain comprises an amino acid sequence set forth in SEQ ID NO:128 and the light chain comprises an animo acid sequence set forth in SEQ ID NO:129. In some aspects, the heavy chain comprises an amino acid sequence set forth in SEQ ID NO:130 and the light chain comprises an animo acid sequence set forth in SEQ ID NO:131. In some aspects, the heavy chain comprises an amino acid sequence set forth in SEQ ID NO:132 and the light chain comprises an animo acid sequence set forth in SEQ ID NO:133. In some aspects, the heavy chain comprises an amino acid sequence set forth in SEQ ID NO:134 and the light chain comprises an animo acid sequence set forth in SEQ ID NO:135. In some aspects, the heavy chain comprises an amino acid sequence set forth in SEQ ID NO:136 and the light chain comprises an animo acid sequence set forth in SEQ ID NO: 137. In some aspects, the heavy chain comprises an amino acid sequence set forth in SEQ ID NO: 138 and the light chain comprises an animo acid sequence set forth in SEQ ID NO:139. In some aspects, the heavy chain comprises an amino acid sequence set forth in SEQ ID NO:140 and the light chain comprises an animo acid sequence set forth in SEQ ID NO: 141. In some aspects, the heavy chain comprises an amino acid sequence set forth in SEQ ID NO: 142 and the light chain comprises an animo acid sequence set forth in SEQ ID NO:143. In some aspects, the heavy chain comprises an amino acid sequence set forth in SEQ ID NO:144 and the light chain comprises an animo acid sequence set forth in SEQ ID NO:145. In some aspects, the heavy chain comprises an amino acid sequence set forth in SEQ ID NO:146 and the light chain comprises an animo acid sequence set forth in SEQ ID NO:147. In some aspects, the heavy chain comprises an amino acid sequence set forth in SEQ ID NO:148 and the light chain comprises an animo acid sequence set forth in SEQ ID NO:149. In some aspects, the heavy chain comprises an amino acid sequence set forth in SEQ ID NO:150 and the light chain comprises an animo acid sequence set forth in SEQ ID NO:151. In some aspects, the heavy chain comprises an amino acid sequence set forth in SEQ ID NO:152 and the light chain comprises an animo acid sequence set forth in SEQ ID NO: 153.

In some aspects, the LTβR antibody comprises FR regions with silent amino acid mutations. “Silent” amino acid mutations refer to a change in an amino acid sequence which does not cause a functional change in the resultant protein function. In some aspects, the LTβR antibody comprises a light chain variable domain with a FR region with a silent amino acid mutation. In some aspects, the LTβR antibody comprises a heavy chain variable domain with a FR region with a silent amino acid mutation. Alternatively, certain amino acid residues in the LTβR antibodies may be any amino acid as notated by “X” in the amino acid sequence. “X” may be any amino acid, including naturally occurring and synthetic amino acids, as well as amino acid analogs and amino acid mimetics that function in a manner similar to the naturally occurring amino acids.

In some aspects, the LTβR antibody or antibody variant is a single chain variable fragment (scFv). Typically, an scFv includes a single light chain and a single heavy chain. For an scFv, each light chain and heavy chain in turn can include two regions: a variable (“V”) region (i.e. variable light chain and variable heavy chain) involved in binding the target antigen, and a constant (“C”) region that interacts with other components of the immune system. An scFv can include a single light chain variable domain or region and a single heavy chain variable domain or region. Also, as noted above, the variable light chain and the variable heavy chain in a single chain antibody may be linked through a linker peptide. Examples for linker peptides of scFvs are provided below in Table 2 and described in Bird, R. E., Hardman, K. D., Jacobson, J. W., Johnson, S., Kaufman, B. M., Lee, S. M., Lee, T., Pope, S. H., Riordan, G. S. and Whitlow, M. (1988).

Methods of making scFv antibodies have been described. See, Huse et al., Science 246:1275-1281 (1989); Ward et al., Nature 341:544-546 (1989); and Vaughan et al., Nature Biotech. 14:309-314 (1996). Briefly, mRNA from B-cells from an immunized animal is isolated and cDNA is prepared. The cDNA is amplified using primers specific for the variable regions of heavy and light chains of immunoglobulins. The PCR products are purified and the nucleic acid sequences are joined. If a linker peptide is desired, nucleic acid sequences that encode the peptide are inserted between the heavy and light chain nucleic acid sequences. The nucleic acid which encodes the scFv is inserted into a vector and expressed in the appropriate host cell.

An scFv that binds to lymphotoxin beta receptor (LTβR) including a light chain variable region and a heavy chain variable region is provided. In some aspects, the scFv binds to human LTβR (SEQ ID NO: 2). The light chain variable region includes a CDR L1, a CDR L2, and a CDR L3 and the heavy chain variable region includes a CDR H1, a CDR H2, and a CDR H3. In some aspects, the CDR L1, CDR L2, and CDR L3 have amino acid sequences as set forth in SEQ ID NOs: 5, 6, and 7 and the CDR H1, CDR H2, and CDR H3 have amino acid sequences as set forth in SEQ ID NOs: 8, 9 and 10. In some aspects, the CDR LI, CDR L2, and CDR L3 have amino acid sequences as set forth in SEQ ID NOs: 13, 14 and 15 and the CDR H1, CDR H2, and CDR H3 have amino acid sequences as set forth in SEQ ID NOs: 16, 17 and 18. In some aspects, the CDR L1, CDR L2, and CDR L3 have amino acid sequences as set forth in SEQ ID NOs: 21, 22, and 23 and the CDR H1, CDR H2, and CDR H3 have amino acid sequences as set forth in SEQ ID NOs: 24, 25 and 26. In some aspects, the CDR L1, CDR L2, and CDR L3 have amino acid sequences as set forth in SEQ ID NOs: 29, 30, and 31 and the CDR H1, CDR H2, and CDR H3 have amino acid sequences as set forth in SEQ ID NOs: 32, 33 and 34. In some aspects, the CDR L1, CDR L2, and CDR L3 have amino acid sequences as set forth in SEQ ID NOs: 37, 38 and 39 and the CDR H1, CDR H2, and CDR H3 have amino acid sequences as set forth in SEQ ID NOs: 40, 41, and 42. In some aspects, the CDR L1, CDR L2, and CDR L3 have amino acid sequences as set forth in SEQ ID NOs: 45, 46 and 47 and the CDR H1, CDR H2, and CDR H3 have amino acid sequences as set forth in SEQ ID NOs: 48, 49 and 50. In some aspects, the CDR L1, CDR L2, and CDR L3 have amino acid sequences as set forth in SEQ ID NOs: 53, 54 and 55 and the CDR H1, CDR H2, and CDR H3 have amino acid sequences as set forth in SEQ ID NOs: 56, 57 and 58. In some aspects, the CDR L1, CDR L2, and CDR L3 have amino acid sequences as set forth in SEQ ID NOs: 61, 62 and 63 and the CDR H1, CDR H2, and CDR H3 have amino acid sequences as set forth in SEQ ID NOs: 64, 65 and 66. In some aspects, the CDR L1, CDR L2, and CDR L3 have amino acid sequences as set forth in SEQ ID NOs: 69, 70 and 71 and the CDR H1, CDR H2, and CDR H3 have amino acid sequences as set forth in SEQ ID NOs: 72, 73 and 74. In some aspects, the CDR L1, CDR L2, and CDR L3 have amino acid sequences as set forth in SEQ ID NOs: 77, 78 and 79 and the CDR H1, CDR H2, and CDR H3 have amino acid sequences as set forth in SEQ ID NOs: 80, 81 and 82. In some aspects, the CDR L1, CDR L2, and CDR L3 have amino acid sequences as set forth in SEQ ID NOs: 85, 86, and 87 and the CDR H1, CDR H2, and CDR H3 have amino acid sequences as set forth in SEQ ID NOs: 88, 89 and 90. In some aspects, the CDR L1, CDR L2, and CDR L3 have amino acid sequences as set forth in SEQ ID NOs: 53, 93 and 94 and the CDR H1, CDR H2, and CDR H3 have amino acid sequences as set forth in SEQ ID NOs: 95, 57 and 58. In some aspects, the CDR L1, CDR L2, and CDR L3 have amino acid sequences as set forth in SEQ ID NOs: 98, 99 and 100 and the CDR H1, CDR H2, and CDR H3 have amino acid sequences as set forth in SEQ ID NOs: 101, 102 and 103. In some aspects, the CDR L1, CDR L2, and CDR L3 have amino acid sequences as set forth in SEQ ID NOs: 108, 109 and 110 and the CDR H1, CDR H2, and CDR H3 have amino acid sequences as set forth in SEQ ID NOs: 111, 112, and 113. In some aspects, the CDR L1, CDR L2, and CDR L3 have amino acid sequences as set forth in SEQ ID NOs: 116, 117 and 118 and the CDR H1, CDR H2, and CDR H3 have amino acid sequences as set forth in SEQ ID NOs: 119, 120 and 121.

In some aspects, the light chain variable region comprises SEQ ID NO:3 and the heavy chain variable region comprises SEQ ID NO:4. In some aspects, the light chain variable region comprises SEQ ID NO: 11 and the heavy chain variable region comprises SEQ ID NO:12. In some aspects, the light chain variable region comprises SEQ ID NO: 19 and the heavy chain variable region comprises SEQ ID NO:20. In some aspects, the light chain variable region comprises SEQ ID NO:27 and the heavy chain variable region comprises SEQ ID NO:28. In some aspects, the light chain variable region comprises SEQ ID NO:35 and the heavy chain variable region comprises SEQ ID NO:36. In some aspects, the light chain variable region comprises SEQ ID NO:43 and the heavy chain variable region comprises SEQ ID NO:44. In some aspects, the light chain variable region comprises SEQ ID NO:51 and the heavy chain variable region comprises SEQ ID NO:52. In some aspects, the light chain variable region comprises SEQ ID NO:59 and the heavy chain variable region comprises SEQ ID NO:60. In some aspects, the light chain variable region comprises SEQ ID NO:67 and the heavy chain variable region comprises SEQ ID NO:68. In some aspects, the light chain variable region comprises SEQ ID NO:75 and the heavy chain variable region comprises SEQ ID NO:76. In some aspects, the light chain variable region comprises SEQ ID NO:83 and the heavy chain variable region comprises SEQ ID NO:84. In some aspects, the light chain variable region comprises SEQ ID NO:91 and the heavy chain variable region comprises SEQ ID NO:92. In some aspects, the light chain variable region comprises SEQ ID NO:96 and the heavy chain variable region comprises SEQ ID NO:97. In some aspects, the light chain variable region comprises SEQ ID NO: 104 and the heavy chain variable region comprises SEQ ID NO:105. In some aspects, the light chain variable region comprises SEQ ID NO: 106 and the heavy chain variable region comprises SEQ ID NO:107. In some aspects, the light chain variable region comprises SEQ ID NO: 114 and the heavy chain variable region comprises SEQ ID NO:115.

The herein provided scFv that binds LTβR is provided in a membrane-bound or membrane-tethered form (e.g., as an mTa). In some aspects, the scFv that binds LTβR further comprises a hinge domain. In some aspects, the hinge domain is a CD28 hinge domain or a CD8 hinge domain. In some aspects, the scFv that binds LTβR further comprises a transmembrane domain. In some aspects, the transmembrane domain is a CD28 transmembrane domain or a CD8 transmembrane domain. Table 1 below provides various hinges and transmembrane domains that can be used in the scFv that binds LTβR described herein. The scFv that binds LTβR can also include a leader sequence or a signal sequence located upstream from the heavy and light chain variable region sequences. For example, the scFv can include a GMCSF signal sequence having the amino acid sequence of MLLLVTSLLLCELPHPAFLLIP (SEQ ID NO: 298).

TABLE 1
Examples of hinges and transmembrane domains.

| Name | Sequence |
|---|---|
| CD28 hinge (36 amino acids) | IEFMYPPPYLDNERSNGTIIHIKEKHLCHTQSSPK |
| | (SEQ ID NO: 165) |
| CD28 hinge (39 amino acids) | IEVMYPPPYLDNEKSNGTIIHVKGKHLCPSPLFPGPS |
| | KP (SEQ ID NO: 166) |
| CD8 hinge (48 amino acids) | AKPTTTPAPRPPTPAPTIASQPLSLRPEACRPAAGGA |
| | VHTRGLDFACD (SEQ ID NO: 167) |
| CD8 hinge (45 amino acids) | TTTKPVLRTPSPVHPTGTSQPQRPEDCRPRGSVKGT |
| | GLDFACDIY (SEQ ID NO: 168) |
| CD28 transmembrane domain (28 | LFWALVVVAGVLFCYGLLVTVALCVIWT (SEQ ID |
| amino acids) | NO: 169) |
| CD28 transmembrane domain (27 | MFWVLVVVGGVLACYSLLVTVAFIIFWV (SEQ ID |
| amino acids) | NO: 170) |
| CD8 transmembrane domain (22 | IWAPLAGICVALLLSLIITLIC (SEQ ID NO: 171) |
| amino acids) | |
| CD8 transmembrane domain (21 | IYIWAPLAGTCGVLLLSLVIT (SEQ ID NO: 172) |
| amino acids) | |
| CD8 transmembrane domain (23 | IYIWAPLAGTCGVLLLSLVITLY (SEQ ID NO: 173) |
| amino acids) | |
| CD8 transmembrane domain (24 | IYIWAPLAGTCGVLLLSLVITLYC (SEQ ID NO: 174) |
| amino acids) | |

In some aspects, the scFv that binds LTβR further comprises a linker sequence. The linker may usually be rich in glycine for flexibility, as well as serine or threonine for solubility. The linker can either connect the N-terminus of the VH with the C-terminus of the VL, or vice versa. Table 2 below provides various linker sequences that can be used in the scFv that binds LTβR described herein.

TABLE 2
Examples of scFv linker sequences.

| Name | Sequence |
|---|---|
| G3 (3 amino acids) | GGG |
| (G4S)3 (15 amino acids) | GGGGSGGGGSGGGGS (SEQ ID NO: 175) |
| GGGSSGGGSG linker (10 amino | GGGSSGGGSG (SEQ ID NO: 176) |
| acids) | |
| GGSAGGG linker (7 amino | GGSAGGG (SEQ ID NO: 178) |
| acids) | |
| GGSAGPAENLYFQGGGGSGG | GGSAGPAENLYFQGGGGSGGGSG (SEQ ID NO: 179) |
| GSG linker (23 amino acids) | |

### Antibodies Binding Mouse LTβR

Also provided herein are lymphotoxin beta receptor (LTβR) antibody or antibody variants which bind to mouse LTβR (SEQ ID NO: 1). In one aspect, a LTβR antibody or antibody variant including a light chain variable region and a heavy chain variable region is provided. The light chain variable region includes a CDR L1, a CDR L2, and a CDR L3 and the heavy chain variable region includes a CDR H1, a CDR H2, and a CDR H3. In some aspects, the CDR L1, CDR L2, and CDR L3 have amino acid sequences as set forth in SEQ ID NOs: 181, 182, and 183, and the CDR H1, CDR H2, and CDR H3 have amino acid sequences as set forth in SEQ ID NOs: 184, 185, and 186. In some aspects, the CDR L1, CDR L2, and CDR L3 have amino acid sequences as set forth in SEQ ID NOs: 189, 190, and 191 and the CDR H1, CDR H2, and CDR H3 have amino acid sequences as set forth in SEQ ID NOs: 192, 193 and 194. In some aspects, the CDR L1, CDR L2, and CDR L3 have amino acid sequences as set forth in SEQ ID NOs: 197, 198, and 199 and the CDR H1, CDR H2, and CDR H3 have amino acid sequences as set forth in SEQ ID NOs: 200, 201, and 202. In some aspects, the CDR L1, CDR L2, and CDR L3 have amino acid sequences as set forth in SEQ ID NOs: 205, 206, and 207 and the CDR H1, CDR H2, and CDR H3 have amino acid sequences as set forth in SEQ ID NOs: 208, 209, 210. In some aspects, the CDR L1, CDR L2, and CDR L3 have amino acid sequences as set forth in SEQ ID NOs: 213, 214, and 215 and the CDR H1, CDR H2, and CDR H3 have amino acid sequences as set forth in SEQ ID NOs: 216, 217, and 218. In some aspects, the CDR L1, CDR L2, and CDR L3 have amino acid sequences as set forth in SEQ ID NOs: 221, 222, and 223 and the CDR H1, CDR H2, and CDR H3 have amino acid sequences as set forth in SEQ ID NOS 224, 225, and 226. In some aspects, the CDR L1, CDR L2, and CDR L3 have amino acid sequences as set forth in SEQ ID NOs: 229, 230, and 231 and the CDR H1, CDR H2, and CDR H3 have amino acid sequences as set forth in SEQ ID NOs: 232, 233, 234. In some aspects, the CDR L1, CDR L2, and CDR L3 have amino acid sequences as set forth in SEQ ID NOs: 237, 238, and 239 and the CDR H1, CDR H2, and CDR H3 have amino acid sequences as set forth in SEQ ID NOs: 240, 241, and 242. In some aspects, the CDR L1, CDR L2, and CDR L3 have amino acid sequences as set forth in SEQ ID NOs: 245, 246, and 247 and the CDR H1, CDR H2, and CDR H3 have amino acid sequences as set forth in SEQ ID NOs: 248, 249, and 250. In some aspects, the CDR L1, CDR L2, and CDR L3 have amino acid sequences as set forth in SEQ ID NOs: 253, 254, and 255 and the CDR H1, CDR H2, and CDR H3 have amino acid sequences as set forth in SEQ ID NOs: 256, 257, and 258.

In some aspects, the light chain variable region comprises SEQ ID NO: 180 and the heavy chain variable region comprises SEQ ID NO:179. In some aspects, the light chain variable region comprises SEQ ID NO:188 and the heavy chain variable region comprises SEQ ID NO: 187. In some aspects, the light chain variable region comprises SEQ ID NO: 196 and the heavy chain variable region comprises SEQ ID NO: 195. In some aspects, the light chain variable region comprises SEQ ID NO:204 and the heavy chain variable region comprises SEQ ID NO: 203. In some aspects, the light chain variable region comprises SEQ ID NO:212 and the heavy chain variable region comprises SEQ ID NO:211. In some aspects, the light chain variable region comprises SEQ ID NO:220 and the heavy chain variable region comprises SEQ ID NO: 219. In some aspects, the light chain variable region comprises SEQ ID NO:228 and the heavy chain variable region comprises SEQ ID NO:227. In some aspects, the light chain variable region comprises SEQ ID NO:236 and the heavy chain variable region comprises SEQ ID NO: 235. In some aspects, the light chain variable region comprises SEQ ID NO:243 and the heavy chain variable region comprises SEQ ID NO:244. In some aspects, the light chain variable region comprises SEQ ID NO:252 and the heavy chain variable region comprises SEQ ID NO: 251.

In another aspect, a LTβR antibody including a heavy chain and a light chain is provided. In some aspects, the light chain comprises SEQ ID NO:260 and the heavy chain comprises SEQ ID NO:259. In some aspects, the light chain comprises SEQ ID NO:262 and the heavy chain comprises SEQ ID NO:261. In some aspects, the light chain comprises SEQ ID NO: 264 and the heavy chain comprises SEQ ID NO:263. In some aspects, the light chain comprises SEQ ID NO:266 and the heavy chain comprises SEQ ID NO:265. In some aspects, the light chain comprises SEQ ID NO:268 and the heavy chain comprises SEQ ID NO:267. In some aspects, the light chain comprises SEQ ID NO:270 and the heavy chain comprises SEQ ID NO: 269. In some aspects, the light chain comprises SEQ ID NO:272 and the heavy chain comprises SEQ ID NO:271. In some aspects, the light chain comprises SEQ ID NO:274, and the heavy chain comprises SEQ ID NO:273. In some aspects, the light chain comprises SEQ ID NO: 276 and the heavy chain comprises SEQ ID NO:275. In some aspects, the light chain comprises SEQ ID NO:279 and the heavy chain comprises SEQ ID NO:278.

An scFv that binds to lymphotoxin beta receptor (LTβR) including a light chain variable region and a heavy chain variable region is provided. In some aspects, the scFv binds to mouse LTβR (SEQ ID NO: 1). Also provided herein are lymphotoxin beta receptor (LTβR) antibody or antibody variants which bind to mouse LTβR (SEQ ID NO: 1). In one aspect, a LTβR antibody or antibody variant including a light chain variable region and a heavy chain variable region is provided. The light chain variable region includes a CDR L1, a CDR L2, and a CDR L3 and the heavy chain variable region includes a CDR H1, a CDR H2, and a CDR H3. In some aspects, the CDR L1, CDR L2, and CDR L3 have amino acid sequences as set forth in SEQ ID NOs: 181, 182, and 183, and the CDR H1, CDR H2, and CDR H3 have amino acid sequences as set forth in SEQ ID NOs: 184, 185, and 186. In some aspects, the CDR L1, CDR L2, and CDR L3 have amino acid sequences as set forth in SEQ ID NOs: 189, 190, and 191 and the CDR H1, CDR H2, and CDR H3 have amino acid sequences as set forth in SEQ ID NOs: 192, 193 and 194. In some aspects, the CDR L1, CDR L2, and CDR L3 have amino acid sequences as set forth in SEQ ID NOs: 197, 198, and 199 and the CDR H1, CDR H2, and CDR H3 have amino acid sequences as set forth in SEQ ID NOs: 200, 201, and 202. In some aspects, the CDR L1, CDR L2, and CDR L3 have amino acid sequences as set forth in SEQ ID NOs: 205, 206, and 207 and the CDR H1, CDR H2, and CDR H3 have amino acid sequences as set forth in SEQ ID NOs: 208, 209, 210. In some aspects, the CDR L1, CDR L2, and CDR L3 have amino acid sequences as set forth in SEQ ID NOs: 213, 214, and 215 and the CDR H1, CDR H2, and CDR H3 have amino acid sequences as set forth in SEQ ID NOs: 216, 217, and 218. In some aspects, the CDR L1, CDR L2, and CDR L3 have amino acid sequences as set forth in SEQ ID NOs: 221, 222, and 223 and the CDR H1, CDR H2, and CDR H3 have amino acid sequences as set forth in SEQ ID NOs 224, 225, and 226. In some aspects, the CDR LI, CDR L2, and CDR L3 have amino acid sequences as set forth in SEQ ID NOs: 229, 230, and 231 and the CDR H1, CDR H2, and CDR H3 have amino acid sequences as set forth in SEQ ID NOs: 232, 233, 234. In some aspects, the CDR L1, CDR L2, and CDR L3 have amino acid sequences as set forth in SEQ ID NOs: 237, 238, and 239 and the CDR H1, CDR H2, and CDR H3 have amino acid sequences as set forth in SEQ ID NOs: 240, 241, and 242. In some aspects, the CDR L1, CDR L2, and CDR L3 have amino acid sequences as set forth in SEQ ID NOs: 245, 246, and 247 and the CDR H1, CDR H2, and CDR H3 have amino acid sequences as set forth in SEQ ID NOs: 248, 249, and 250. In some aspects, the CDR LI, CDR L2, and CDR L3 have amino acid sequences as set forth in SEQ ID NOs: 253, 254, and 255 and the CDR H1, CDR H2, and CDR H3 have amino acid sequences as set forth in SEQ ID NOs: 256, 257, and 258.

In some aspects, the light chain variable region comprises SEQ ID NO: 180 and the heavy chain variable region comprises SEQ ID NO:179. In some aspects, the light chain variable region comprises SEQ ID NO:188 and the heavy chain variable region comprises SEQ ID NO: 187. In some aspects, the light chain variable region comprises SEQ ID NO:196 and the heavy chain variable region comprises SEQ ID NO:195. In some aspects, the light chain variable region comprises SEQ ID NO:204 and the heavy chain variable region comprises SEQ ID NO: 203. In some aspects, the light chain variable region comprises SEQ ID NO:212 and the heavy chain variable region comprises SEQ ID NO:211. In some aspects, the light chain variable region comprises SEQ ID NO:220 and the heavy chain variable region comprises SEQ ID NO: 219. In some aspects, the light chain variable region comprises SEQ ID NO:228 and the heavy chain variable region comprises SEQ ID NO:227. In some aspects, the light chain variable region comprises SEQ ID NO:236 and the heavy chain variable region comprises SEQ ID NO: 235. In some aspects, the light chain variable region comprises SEQ ID NO:243 and the heavy chain variable region comprises SEQ ID NO:244. In some aspects, the light chain variable region comprises SEQ ID NO:252 and the heavy chain variable region comprises SEQ ID NO: 251.

The herein provided scFv that binds mouse LTβR is provided in a membrane-bound or membrane-tethered form (e.g., as an mTa). In some aspects, the scFv that binds LTβR further comprises a hinge domain. In some aspects, the hinge domain is a CD28 hinge domain or a CD8 hinge domain. In some aspects, the scFv that binds LTβR further comprises a transmembrane domain. In some aspects, the transmembrane domain is a CD28 transmembrane domain or a CD8 transmembrane domain. Table 1 above provides various hinges and transmembrane domains that can be used in the scFv that binds LTβR described herein. In some aspects, the scFv that binds LTβR further comprises a linker sequence. The linker may usually be rich in glycine for flexibility, as well as serine or threonine for solubility. The linker can either connect the N-terminus of the VH with the C-terminus of the VL, or vice versa. Table 2 above provides various linker sequences that can be used in the scFv that binds LTβR described herein.

### Nucleic Acids

In one aspect, a nucleic acid encoding a LTβR antibody or scFv that binds LTβR is provided herein. The LTβR antibodies and scFv that binds LTβR encoded by the nucleic acid is described in detail throughout this application (including the description above and in the examples section). Thus, the LTβR antibody or scFv that binds LTβR encoded by the isolated nucleic acid includes all of the some aspects described herein. For example, the nucleic acid may encode at least one CDR, specific residues involved in binding the epitope, or binding framework residues. For instance, the nucleic acid may encode a light chain including a sequence of SEQ ID NO:8. As another example, the nucleic acid encoding a scFv that binds LTβR may include a hinge domain. For instance, the nucleic acid may encode a hinge including a sequence of SEQ ID NO: 165. In some aspects, the nucleic acid includes the sequence of SEQ ID NO: 154.

In some aspects, the nucleic acid encoding a LTβR antibody or scFv that binds LTβR is provided in a vector. Such a vector can be chosen from viral vectors and non-viral vectors, plasmids, cosmids, transposons, and artificial chromosomes. By way of example, the vector can be a viral vector, such as a lentiviral vector, a retroviral vector, an adenoviral vector, or an adeno-associated viral vector. The vector optionally comprises nucleic acid sequences that encode transposases and/or nucleases. Non-viral vector examples include physical vectors such as electroporation and chemical vectors such as lipid nanoparticles.

### Pharmaceutical Compositions

A pharmaceutical composition including a LTβR antibody or scFv that binds LTβR provided herein and a pharmaceutically acceptable excipient is provided. The pharmaceutical composition can include a therapeutically effective amount the antibody or scFv.

A therapeutically effective amount as provided herein refers to an amount effective to achieve its intended purpose. The actual amount effective for a particular application will depend, inter alia, on the condition being treated When administered in methods to treat a disease, the pharmaceutical compositions described herein will contain an amount of active antibody or scFv that binds LTβR effective to achieve the desired result, e.g., modulating the activity of a target molecule (e.g., LTβR), inducting tertiary lymphoid structure formation, and/or reducing, eliminating, or slowing the progression of disease symptoms (e.g., cancer). Determination of a therapeutically effective amount of a LTβR antibody or scFv that binds LTβR provided herein is well within the capabilities of those skilled in the art, especially in light of the detailed disclosure herein.

Acceptable carriers, excipients or stabilizers are nontoxic to recipients at the dosages and concentrations employed, and include buffers such as phosphate, citrate, or acetate at a pH typically of 5.0 to 8.0, most often 6.0 to 7.0; salts such as sodium chloride, potassium chloride, etc. to make isotonic; antioxidants, preservatives, low molecular weight polypeptides, proteins, hydrophilic polymers such as polysorbate 80, amino acids such as glycine, carbohydrates, chelating agents, sugars, and other standard ingredients known to those skilled in the art (Remington: The Science and Practice of Pharmacy, Mack Publishing Co., 20th ed., 2020). The antibody or antibody variant is typically present at a concentration of 0.1-100 mg/ml, e.g., 1-10 mg/ml or 10-50 mg/ml, for example 5, 10, 20, 30, 40, 50 or 60 mg/ml.

A pharmaceutical composition including an antibody or antibody variant (e.g., scFv) as described herein can be administered by a variety of methods known in the art. The route and/or mode of administration vary depending upon the desired results. In some aspects, administration is intravenous, intramuscular, intraperitoneal, or subcutaneous, or administered proximal to the site of the target. Pharmaceutically acceptable excipients can be suitable for intravenous, intramuscular, subcutaneous, parenteral, spinal or epidermal administration (e.g., by injection or infusion).

Pharmaceutical compositions of the antibody or scFv that binds LTβR can be prepared in accordance with methods well known and routinely practiced in the art. See, e.g., Remington: The Science and Practice of Pharmacy, Mack Publishing Co., 20th ed., 2020; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the antibody or antibody variant is employed in the pharmaceutical compositions of the invention. The antibodies or antibody variants provided can be formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It may be advantageous to formulate the antibodies or antibody variants in combination with other therapies or agents. It can be advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of antibody or antibody variant calculated to produce the desired therapeutic effect in association with the required pharmaceutical excipient.

Actual dosage levels of the active ingredients in the pharmaceutical compositions of the present invention can be varied to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, the route of administration, the time of administration, the rate of excretion of the particular antibody being employed, the duration of the treatment, other drugs, compounds and/or materials used in combination with the particular compositions employed, the age, sex, weight, condition, general health and prior medical history of the patient being treated, and like factors.

A physician or veterinarian can start doses of the antibodies or antibody variants of the present disclosure employed in the pharmaceutical composition at levels lower than that required to achieve the desired therapeutic effect and gradually increase the dosage until the desired effect is achieved. In general, effective doses of the compositions of the present invention vary depending upon many different factors, including the specific disease or condition to be treated, means of administration, target site, physiological state of the patient, whether the patient is human or an animal, other medications administered, and whether treatment is prophylactic or therapeutic. Treatment dosages need to be titrated to optimize safety and efficacy. For administration with an antibody, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months.

The LTβR antibody or scFv that binds LTβR provided herein can be administered on multiple occasions. Intervals between single dosages can be weekly, monthly or yearly. Intervals can also be irregular as indicated by measuring blood levels of the humanized antibody in the patient. In some methods, dosage is adjusted to achieve a plasma antibody concentration of 1-1000 μg/ml and in some methods 25-300 μg/ml. Alternatively, antibody or antibody variant can be administered as a sustained release formulation, in which case less frequent administration is required. Dosage and frequency vary depending on the half-life of the antibody or antibody variant in the patient. In general, humanized antibodies show longer half-life than that of chimeric antibodies and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### Cell Compositions

Also described herein is a population of human T cells comprising the scFv that binds LTβR described herein. In some aspects, the population of human T cells are transduced by a vector comprising an expression cassette comprising a nucleic acid molecule encoding the scFv that binds LTβR described herein. Optionally, the T cells comprise CD4+ helper T cells and CD8+ memory T cells. Optionally, the T cells comprise CD4+ helper T cells and CD8+ helper T cells.

In some aspects, the population of human T cells further comprise a chimeric antigen receptor (CAR). CARs are composed of an extracellular recognition/targeting domain, an extracellular hinge domain, a transmembrane domain, and intracellular signaling and co-stimulatory domains. Table 3 below provides components which may be useful in the population of human T cells further comprising a CAR described herein.

TABLE 3
Components of CARs.

| Component | Sequence |
|---|---|
| A3 spacer | AAA |
| linker | GGGSSGGGSG (SEQ ID NO: 279) |
| IgG4 hinge (S→P) | ESKYGPPCPPCP (SEQ ID NO: 280) |
| (S228P) | |
| IgG4 hinge | ESKYGPPCPSCP (SEQ ID NO: 281) |
| IgG4 hinge (S228P) + linker | ESKYGPPCPPCPGGGSSGGGSG (SEQ ID NO |
| | 282:) |
| CD28 hinge | IEVMYPPPYLDNEKSNGTIIHVKGKHL |
| | CPSPLFPGPSKP (SEQ ID NO: 166) |
| CD8 hinge-48aa | AKPTTTPAPRPPTPAPTIASQPLSLRPEACR |
| | PAAGGAVHTRGLDFACD (SEQ ID |
| | NO: 167) |
| CD8 hinge-45aa | TTTPAPRPPTPAPTIASQPLSLRPEACR |
| | PAAGGAVHTRGLDFACD (SEQ ID |
| | NO: 168) |
| IgG4(HL-CH3) | ESKYGPPCPPCPGGGSSGGGSGGQPR |
| (includes S228P in hinge) | EPQVYTLPPSQEEMTKNQVSLTCLVK |
| | GFYPSDIAVEWESNGQPENNYKTTPP |
| | VLDSDGSFFL YSRLTVDKSRWQEGNV |
| | FSCSVMHEALHNHYTQKSLSLSLGK |
| | (SEQ ID NO: 283) |
| IgG4(L235E,N297Q) | ESKYGPPCPSCPAPEFEGGPSVFLFPPK |
| | PKDTLMISRTPEVTCVVVDVSQEDPE |
| | VQFNWYVDGVEVHQAKTKPREEQF |
| | STYRVVSVLTVLHQDWLNGKEYKCK |
| | VSNKGLPSSIEKTISKAKGQPREPQVY |
| | TLPPSQEEMTKNQVSLTCLVKGFYPS |
| | DIAVEWESNGQPENNYKTTPPVLDSD |
| | GSFFLYSRLTVDKSRWQEGNVFSCSV |
| | MHEALHNHYTQKSLSLSLGK (SEQ ID NO: |
| | 284) |
| IgG4(S228P, L235E, N297Q) | ESKYGPPCPPCPAPEFEGGPSVFLFPPK |
| | PKDTLMISRTPEVTCVVVDVSQEDPE |
| | VQFNWYVDGVEVHQAKTKPREEQFQ |
| | STYRVVSVLTVLHQDWLNGKEYKCK |
| | VSNKGLPSSIEKTISKAKGQPREPQVY |
| | TLPPSQEEMTKNQVSL TCLVKGFYPS |
| | DIAVEWESNGQPENNYKTTPPVLDSD |
| | GSFFLYSRLTVDKSRWQEGNVFSCSV |
| | MHEALHNHYTQKSLSLSLGK (SEQ ID |
| | NO: 285) |
| IgG4(CH3) | GQPREPQVYTLPPSQEEMTKNQVSLT |
| | CLVKGFYPSDIAVEWESNGQPENNYK |
| | TTPPVLDSDGSFFLYSRLTVDKSRWQ |
| | EGNVFSCSVMHEALHNHYTQKSLSLSLGK |
| | (SEQ ID NO: 286) |
| CD3zeta transmembrane domain | LCYLLDGILFIYGVILTALFL (SEQ ID NO: |
| | 287) |
| CD28 transmembrane domain (27 amino | FWVLVVVGGVLACYSLLVTVAFIIFWV (SEQ |
| acid) | ID NO: 170) |
| CD28 transmembrane domain (28 amino | MFWVLVVVGGVLACYSLLVTVAFIIFWV |
| acid) | (SEQ ID NO: 169) |
| CD4 transmembrane domain | MALIVLGGVAGLLLFIGLGIFF (SEQ ID NO: |
| | 288) |
| CD8 transmembrane domain (22 amino | IWAPLAGICVALLLSLIITLIC (SEQ ID NO: |
| acids) | 171) |
| CD8 transmembrane domain (21 amino | IYIWAPLAGTCGVLLLSLVIT (SEQ ID NO: |
| acids) | 172) |
| CD8 transmembrane domain (23 amino | IYIWAPLAGTCGVLLLSLVITLY (SEQ ID NO: |
| acids) | 173) |
| CD8 transmembrane domain (24 amino | IYIWAPLAGTCGVLLLSLVITLYC (SEQ ID |
| acids) | NO: 289) |
| 41BB transmembrane domain | IISFFLALTSTALLFLLFFLTLRFSVV (SEQ ID |
| | NO: 290) |
| CD3zeta instracellular signaling | RVKFSRSADAPAYQQGQNQLYNELNLGR |
| domain | REEYDVLDKRRGRDPEMGGKPRRKNPQ |
| | EGLYNELQKDKMAEAYSEIGMKGERRR |
| | GKGHDGLYQGLSTATKDTYDALHMQALPPR |
| | (SEQ ID NO: 291) |
| CD28 costimulatory domain | RSKRSRLLHSDYMNMTPRRPGPTRKHYQ |
| | PYAPPRDFAAYRS (SEQ ID NO: 292) |
| CD28gg* costimulatory domain | RSKRSRQHSDYMNMTPRRPGPTRKHY |
| | QPYAPPRDFAAYRS (SEQ ID NO: 293) |
| 41BB costimulatory domain | KRGRKKLLYIFKQPFMRPVQTTQEEDGC |
| | SCRFPEEEEGGCEL (SEQ ID NO: 294) |
| OX40 costimulatory domain | ALYLLRRDQRLPPDAHKPPGGGSFRTPIQ |
| | EEQADAHSTLAKI (SEQ ID NO: 295) |

The majority of CAR tumor targeting domains are single chain variable fragments (scFvs) derived from antibody sequences that exploit the specificity of antibody binding to particular antigens. There are also examples of CAR tumor targeting domains derived from normal receptor ligands, such as the IL-13 cytokine CAR that targets cells expressing the IL-13 receptor, IL13Ra2. In some aspects, the CAR comprises a HER2 targeting sequence. In some aspects, the CAR comprises a CD19 targeting sequence. In some aspects, the CAR comprises an Interleukin Receptor targeting sequence. In some aspects, the Interleukin Receptor targeting sequence targets interleukin-13 receptor subunit alpha-2 (IL13Ra2). In some aspects, the CAR comprises an interleukin. In some aspects, the interleukin is IL13.

Suitable extracellular hinge and transmembrane domains for use in the CAR are provided above in Table 1. In some aspects, the CAR comprises an extracellular hinge domain selected from CD8 or CD28. In some aspects, the CAR comprises an extracellular hinge domain comprising SEQ ID NO: 168. In some aspects, the CAR comprises a transmembrane domain selected from CD8 or CD28. In some aspects, the CAR comprises a transmembrane domain comprising SEQ ID NO: 171.

In some aspects, the extracellular hinge and transmembrane domain used in the CAR are derived from a different protein than the extracellular hinge and transmembrane domain used in the scFv that binds LTβR to prevent dimerization. For example, the scFv that binds LTβR may comprise a CD28 (SEQ ID NO: 165) extracellular hinge and CD28 transmembrane domain (SEQ ID NO: 169) and the CAR may comprise a CD8 (SEQ ID NO: 168) extracellular hinge and CD8 transmembrane domain (SEQ ID NO: 171). Alternatively, the scFv that binds LTβR may comprise a CD8 (SEQ ID NO: 168) extracellular hinge and CD8 transmembrane domain (SEQ ID NO: 171) and the CAR may comprise a CD28 (SEQ ID NO: 165) extracellular hinge and CD28 transmembrane domain (SEQ ID NO: 169).

In some aspects, the CAR comprises a linker sequence. Suitable linker sequences for use in the CAR are provided above in Table 2. In some aspects, the CAR comprises a linker sequence comprising SEQ ID NO: 174.

In some aspects, the CAR comprises an intracellular signaling domain derived from the zeta chain of the human CD3 complex (CD3ζ) and one or more costimulatory domains, e.g., a 4-1BB (CD137), CD28, or inducible T-cell costimulatory (ICOS; CD278) costimulatory domain. Intracellular signaling protein CD3ζ contains three immunoreceptor tyrosine-based activation motif (ITAM) domains. ITAM domains are conserved sequences of four amino acids in the cytoplasmic domain of signaling proteins (e.g., CD3ζ). It is thought that restricting ITAM domains to one or two functional ITAM domains enhances CAR signaling and function. Thus, in some aspects, the CAR comprises a CD3ζ intracellular signaling domain comprising one or two deactivated (i.e., nonfunctional) ITAM domains.

Exemplary CARs that can be used have been described and may comprise any of the nucleic acid or amino acid sequences described in U.S. Pat. Nos. 10,676,717 and 11,197,919, and U.S. patent application Ser. No. 18/266,443, each of which are incorporated herein by reference in their entirety.

### Methods for Treating Cancer

Also provided herein are methods for treating cancer in a subject comprising administering to the subject a therapeutically effective amount of the pharmaceutical composition described herein comprising a LTβR antibody or scFv that binds LTβR or a therapeutically effective amount of the population of human T cells described herein comprising a scFv that binds LTβR and optionally a CAR. In some aspects, the cancer is lung cancer, breast cancer, ovarian cancer, uterine cancer, prostate cancer, testicular cancer, kidney cancer, urinary tract cancer, oral cancer, head and neck cancer, esophageal cancer, gastric cancer, pancreatic cancer, colorectal cancer, skin cancer, melanoma, sarcoma, lymphoma, leukemia, or brain cancer including glioblastoma, anaplastic astrocytoma, oligodendroglioma, and medulloblastoma. In some aspects, the cancer is a glioblastoma.

In some aspects, the method comprises administering to the subject a second therapeutic agent. In some aspects, the second therapeutic agent is an immune checkpoint inhibitor. In some aspects, the immune checkpoint inhibitor is an anti-PD-1 or anti-PD-L1 antibody, an anti-CTLA4 antibody, an anti-LAG3 antibody, or the like.

The therapeutically effective amount of the pharmaceutical composition comprising a LTβR antibody or scFv that binds LTβR or the therapeutically effective amount of the population of human T cells comprising a scFv that binds LTβR and optionally a CAR may be administered by a variety of methods known in the art described herein. The route and/or mode of administration vary depending upon the desired results. For example, the therapeutically effective amount of the pharmaceutical composition comprising a LTβR antibody or scFv that binds LTβR or the therapeutically effective amount of the population of human T cells comprising a scFv that binds LTβR and optionally a CAR may be administered intravenously, in single, or multiple occasions.

In methods for treating cancer in a subject comprising administering to the subject a therapeutically effective amount of the population of human T cells described herein comprising a scFv that binds LTβR and optionally a CAR, the population of T cells may be autologous or allogeneic to the patient. In some aspects, the population of human T cells comprise CD4+TN cell and CD8+ TCM cells. The cells provided can be formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It may be advantageous to formulate the cells in combination with other therapies or agents. It can be advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of humanized antibody calculated to produce the desired therapeutic effect in association with the required pharmaceutical excipient.

By way of example, cells can be administered to a subject by absolute numbers of cells, e.g., said subject can be administered from about 1000 cells/injection to up to about 10 billion cells/injection, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) cells per injection, or any ranges between any two of the numbers, end points inclusive. Optionally, from 1×10⁶ to 1×10⁸ cells are administered to the subject. Optionally, the cells are administered one or more times weekly for one or more weeks. Optionally, the cells are administered once or twice weekly for 1, 2, 3, 4, 5, 6, 7, 8, 9, 10 or more weeks.

Optionally, subject are administered from about 1000 cells/injection/m² to up to about 10 billion cells/injection/m², such as at about, at least about, or at most about, 1×10⁸/m², 1×10⁷/m², 5×10⁷/m², 1×10⁶/m², 5×10⁶/m², 1×10⁵/m², 5×10⁵/m², 1×10⁴/m², 5×10⁴/m², 1×10³/m², 5×10³/m² (and so forth) cells per injection, or any ranges between any two of the numbers, end points inclusive.

Optionally, the total dose may calculated by m² of body surface area, including about 1×10¹¹, 1×10¹⁰, 1×10⁹, 1×10⁸, 1×10⁷, per m², or any ranges between any two of the numbers, end points inclusive. Optionally, between about 1 billion and about 3 billion cells are administered to a patient. Optionally, the amount of cells injected per dose may calculated by m² of body surface area, including 1×10¹¹, 1×10¹⁰, 1×10⁹, 1×10⁸, 1×10⁷, per m².

Optionally, cells are administered in a composition comprising the cells and a medium, such as human serum or an equivalent thereof. Optionally, the medium comprises human serum albumin. Optionally, the medium comprises human plasma. Optionally, the medium comprises about 1% to about 15% human serum or human serum equivalent. Optionally, the medium comprises about 1% to about 10% human serum or human serum equivalent. Optionally, the medium comprises about 1% to about 5% human serum or human serum equivalent. Optionally, the medium comprises about 2.5% human serum or human serum equivalent. Optionally, the serum is human AB serum. Optionally, a serum substitute that is acceptable for use in human therapeutics is used instead of human serum. Such serum substitutes may be known in the art. Optionally, the cells are administered in a composition comprising the cells and an isotonic liquid solution that supports cell viability. Optionally, the cells are administered in a composition that has been reconstituted from a cryopreserved sample.

### Methods for Making Cells

Also provided herein are methods for making a population of T cells expressing a scFv that binds LTβR described herein. In some aspects, the methods for making a population of T cells expressing a scFv that binds LTβR comprise (i) obtaining a population of T cells from a subject, (ii) transducing the population of T cells with a nucleic acid sequence encoding the scFv that binds LTβR, and (iii) isolating the population of T cells expressing the scFv that binds LTβR. In some aspects, the population of T cells may comprise CD4+TN cell and CD8+ TCM cells. Also provided are cells made by the methods of making described herein.

In some aspects, the population of T cells are transduced with a vector comprising a nucleic acid sequence encoding the scFv that binds LTβR. In some aspects, the vector is chosen from viral vectors and non-viral vectors, plasmids, cosmids, transposons, and artificial chromosomes. By way of example, the vector can be a viral vector, such as a lentiviral vector, a retroviral vector, an adenoviral vector, or an adeno-associated viral vector. The vector optionally comprises nucleic acid sequences that encode transposases and/or nucleases. Non-viral vector examples include physical vectors such as electroporation and chemical vectors such as lipid nanoparticles.

In some aspects, the scFv that binds LTβR further comprises a hinge domain as discussed above. In some aspects, the scFv that binds LTβR further comprises a transmembrane domain as discussed above. In some aspects, the scFv that binds LTβR further comprises a linker as discussed above. In some aspects, the scFv that binds LTβR comprises the nucleic acid sequence of SEQ ID NO: 154. In some aspects, the scFv that binds LTβR comprises the amino acid sequence of SEQ ID NO: 155.

In some aspects, the methods for making a population of T cells expressing a scFv that binds LTβR further comprise modifying the population of T cells to express a CAR described above and elsewhere herein. In some aspects, the CAR comprises a HER2 targeting sequence. In some aspects, the CAR comprises a CD19 targeting sequence. In some aspects, the CAR comprises an Interleukin Receptor targeting sequence. In some aspects, the Interleukin Receptor targeting sequence targets interleukin-13 receptor subunit alpha-2 (IL13Ra2). In some aspects, the CAR comprises an Interleukin Receptor targeting sequence comprises SEQ ID NO: 160. In some aspects, the CAR comprises an interleukin. In some aspects, the interleukin is IL13. In some aspects, the IL13 comprises SEQ ID NO: 160.

### EXAMPLES

### Example 1: LTβR Ligation Activates NFκB Processing

LTβR ligation activates the non-canonical NFkB pathway to induce the expression of genes vital for lympho-neogenesis. These include C-C motif chemokine ligand 19 (CCL19), CCL21, and C-X-C motif chemokine ligand 13 (CXCL13). The cognate receptor for both CCL19 and CCL21 is C-C chemokine receptor type 7 (CCR7) expressed by naïve T cells and DC. Contrarily, the cognate receptor of CXCL13 is C-X-C chemokine receptor type 5 (CXCR5) expressed on follicular T helper cells and B cells. Therefore, TLS formation requires CXCL13 to recruit and mediate the organization of B cell follicles, while CCL19 and CCL21 to recruit and organize T cell zones surrounding these follicles. To optimize a model for tertiary lymphoid structure (TLS) induction via LTβR ligation, commercially available antibodies were screened. Recombinant rat anti-mouse 5G11 antibody (Creative Biolabs) and anti-human 1D1C5C8 (Thermofisher) were crosslinked onto Dynabeads Protein G. Murine cell line NIH3T3 cells and human cell line HT1080 cells were treated with antibody-coated beads for 24 hours. Birinapant was used as a positive control for NFkB processing. Cell lysate was collected and stained for P100/P52 to measure NFkB Signaling. Secondary antibody staining was detected using ChemiDoc Imaging System (Bio-Rad).

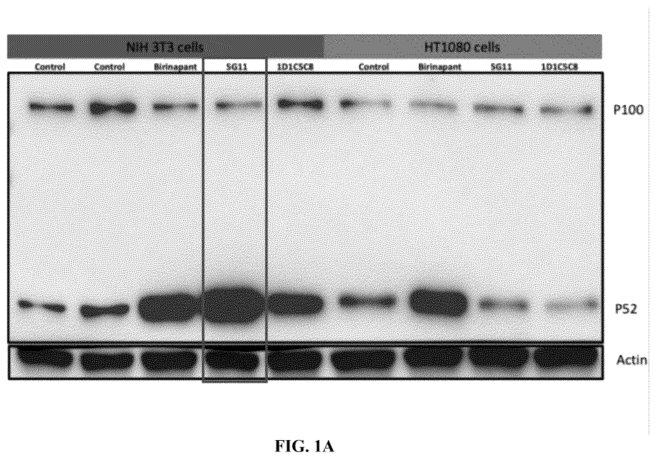

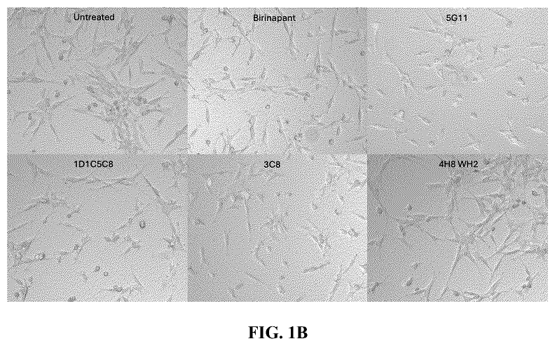

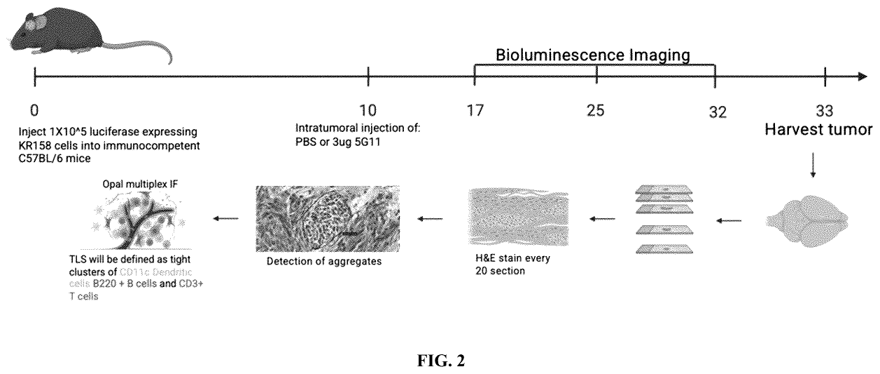

Neither 5G11 antibody nor 1D1C5C8 were cross reactive. The experiment was repeated without crosslinking antibodies to beads and instead treating NIH3T3 and HT1080 cells with 100 ng of antibody for 24 hours. As shown in FIG. 1, only 5G11 processed P100 in NIH3T3 cells highlighted by a rectangle. 5G11 was used for later in vivo experiments.

### Example 2: Detecting and Analyzing Tertiary Lymphoid Structures

Emerging evidence underscores the prognostic value of TLS in certain solid tumors, including brain tumors, in correlation to improved outcome and response to immunotherapy. These correlative studies depended on nuclear based morphological features to detect and quantify TLS from Hematoxylin and Eosin (H&E) staining of patient samples. TLS from H&E stains are defined as tight and dense clusters of more than 50 lymphocytes. However, H&E staining does not accurately depict the maturity and composition of TLS. To this end, both H&E and opal multiplex Immunohistochemistry (IHC) were used to detect and distinguish lymphoid aggregates (as clusters of B and T cells without any defined zones), primary TLS (organized and dense B and T zones), and secondary TLS (presentation of germinal center).

The experimental design is depicted in FIG. 2. Briefly, C57BL/6 mice were treated with PBS (control) or 0.15 ug/g of 5G11 antibody (treated) 10 days after orthotopic KR158-Luciferase tumor engraftment via intracranial delivery. To monitor tumor growth, the mice were imaged every week. 23 days post treatment, mice were euthanized, and brains were harvested after cardiac perfusion with PBS followed by fixation in 4% PFA. 200 Formalin-fixed, paraffin-embedded 10 μm axial brain sections were prepared and every 20th section was H&E stained. Immune infiltration and aggregation in several H&E slides were annotated and used to train the pixel classifier in QuPath to define lymphoid aggregates. Subsequent slides of aggregate positive H&E slides were IHC stained for CD45 (leukocytes), CD3 (T cells), B220 (B cells), and CD11c (dendritic cells).

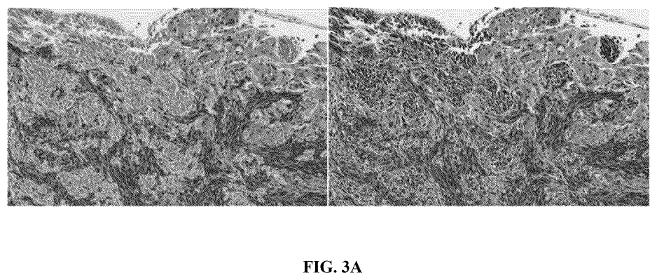

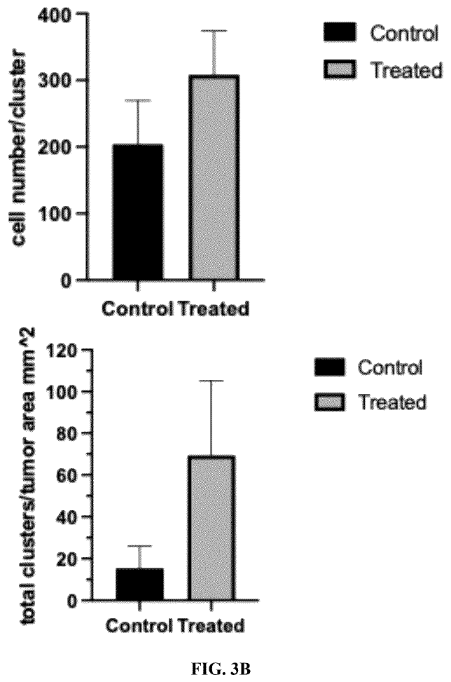

The trained pixel classifier was applied on all H&E stains from both the PBS treated control group and antibody treated group (FIG. 3A). Aggregates were defined as a minimum of 50 lymphoid cells and the total number of aggregates/tumor area in mm², and the cell number/cluster were graphed. The treated group had a 4.6-fold difference in total number of aggregates (FIG. 3B).

### Example 3: Locoregional 5G11 Treatment Improves B and T Cell Infiltration

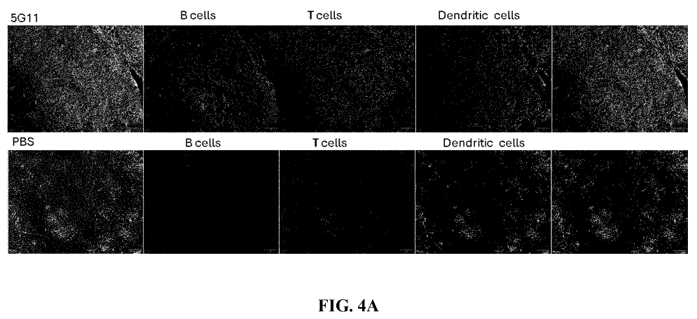

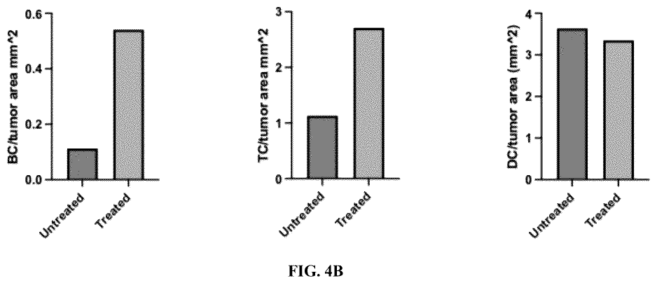

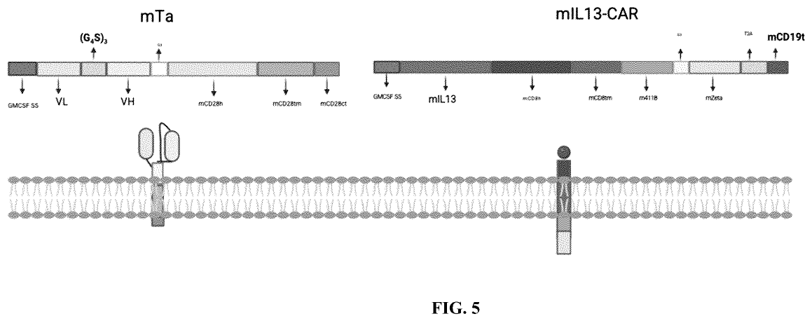

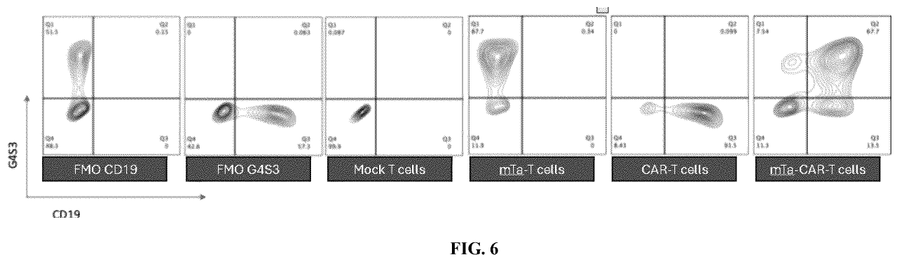

The experiments were according to the methods of Example 2, depictured in FIG. 2. Using the object classifier in Qupath, the total number of B cells, T cells, and dendritic cells were quantified from the IHC stained slides (FIG. 4A). Comparing tumors between the control and treated group, the antibody treated group had greater B and T cell infiltration/tumor area (mm²) (FIG. 4B).

### Example 4: Sequencing 5G11

Lymphotoxin-α1β2 (LTα1β2)-LTβR ligation signaling axis reinforces lymphoid inducer cells (LTi cells), those that express LTα1β2, to maintain a chemokine gradient needed for the organization and maturation of TLS. As such, a membrane-tethered scFv that binds LTβR was formed. As 5G11 was the only commercially available antibody that processed P100 in NIH3T3 cells (FIG. 1), 5G11 was sequenced for later scFv construction. 5G11 was sequenced by The Translational Genomics Research Institute (TGen) using multiple enzyme digestion, LC-MS/MS, de-novo sequence interpretation, and assembly of the sequence on a scaffold from existing mouse monoclonal antibodies.

The light chain and heavy chain sequences are shown below, with confirmed CDR sequences in bold.

LC Light Chain IGKV16S1*01 IGKJ2-3*01 IGKC*01
(SEQ ID NO: 296)
DVQMTQSPSYLAASPGESVSIRCKASKSISNNLAWYQEKPGKANKLLIHS
GSTLQSGTPSRFSGSGSGTDFTLTIRSLEFEDFAVYYCQQYNEYPYTFGA
GTKLELKRADAAPTVSIFPPSTEQLATGGASVVCLMNNFYPRDISVKWKI
DGTERRDGVLDSVTDQDSKDSTYSMSSTLSLTKADYESHNLYTCEVVHKT
SSSPVVKSFNRNEC
HC Heavy Chain IGHV2S18*01 IGHJ1*01 IGHG2A*01
(SEQ ID NO: 297)
QVQLKESGPGLVQPSQTLSLTCTVSGFSLTTYSVHWVRQHSGKSLEWMGR
MWTDGDTSYNSAFTSRLSISRDTSKSQVFLKMNSLQTEDTGTYYCARGYW
YFDFWGPGTMVTVSSAETTAPSVYPLAPGTALKSNSMVTLGCLVKGYFPE
PVTVTWNSGALSSGVHTFPAVLQSGLYTLTSSVTVPSSTWSSQAVTCNVA
HPASSTKVDKKIVPRECNPCGCTGSEVSSVFIFPPKTKDVLTITLTPKVT
CVVVDISQNDPEVRFSWFIDDVEVHTAQTHAPEKQSNSTLRSVSELPIVH
RDWLNGKTFKCKVNSGAFPAPIEKSISKPEGTPRGPQVYTMAPPKEEMTQ
SQVSITCMVKGFYPPDIYTEWKMNGQPQENYKNTPPTMDTDGSYFLYSKL
NVKKETWQQGNTFTCSVLHEGLHNHHTEKSLSHSPGK

### Example 5: LTβR Antibody Generation

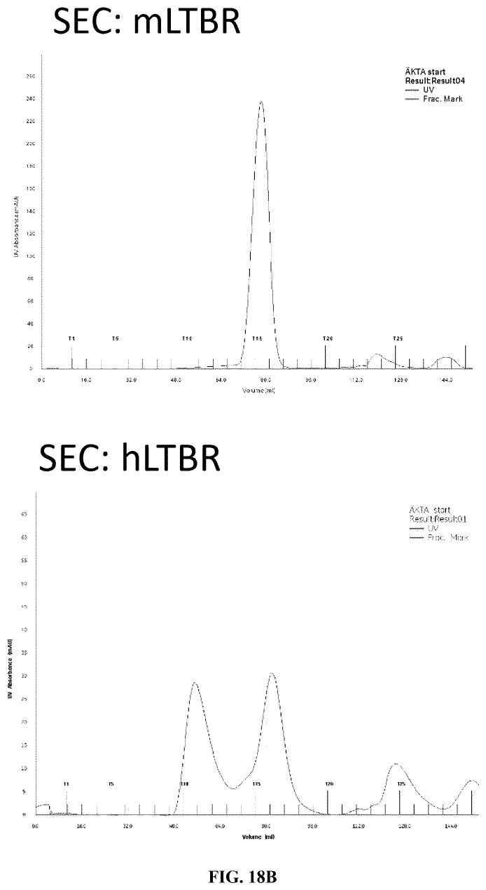

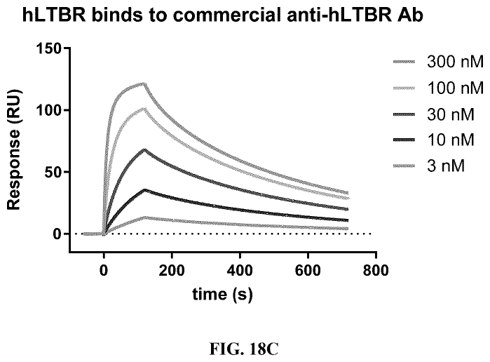

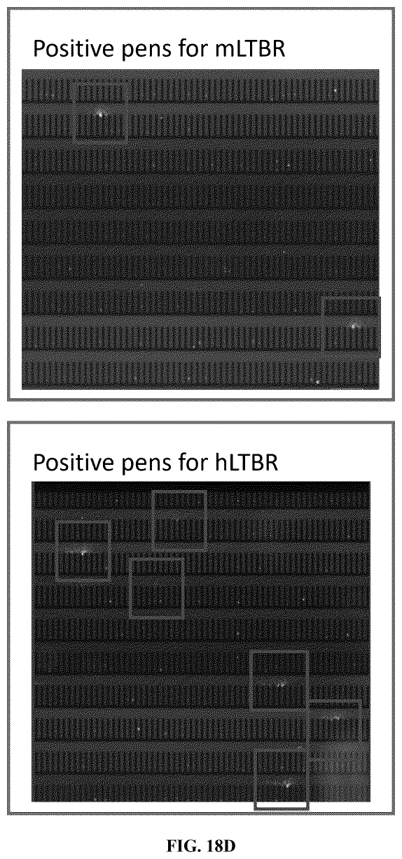

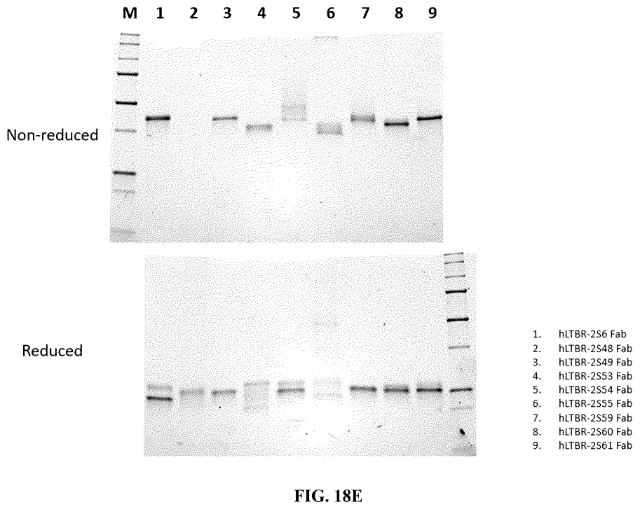

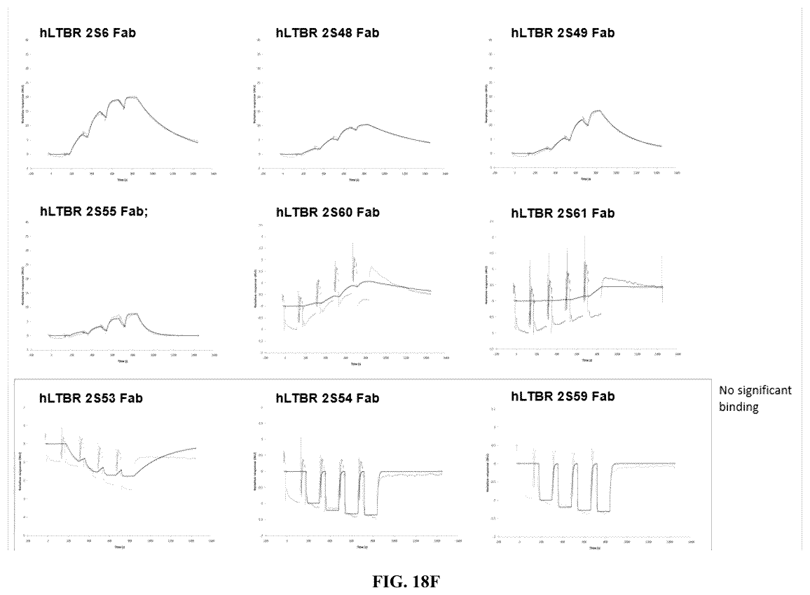

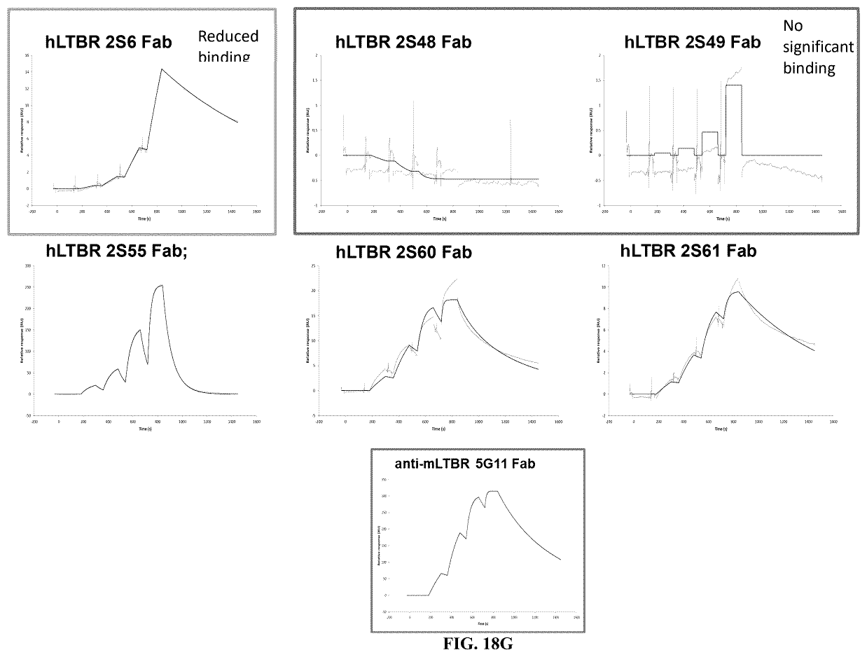

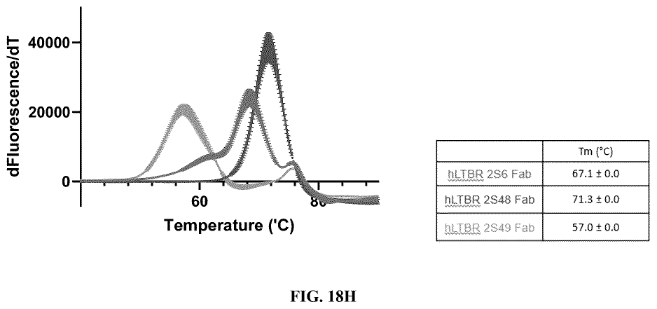

To develop an anti-LTβR antibody, rabbits were immunized with the mouse or human LTβR amino acid sequence (SEQ ID NO: 1 or 2) (FIG. 18A-C). Memory B cells from spleens of rabbits were screened for LTβR antibody production using the Beacon® optofluidic system. Cells secreting anti-LTβR antibodies were identified, and their variable heavy (VH) and light (VL) chain sequences were determined from reverse transcription and cDNA synthesis. Ten sets of mouse LTβR VH/VL sequences and sixteen sets of human LTβR VH/VL sequences were identified (FIG. 18D). VH/VL sequences were cloned into a vector containing human constant domains to create rabbit-human and mouse-human chimeric anti-LTβR antibodies.

### Example 6: Design and Validation of mTa and Murine mTa-CAR T Cells

As shown in FIG. 5, a membrane-tethered agonist (mTa) using an scFv from VH/VL sequences 5G11 was constructed. IL13Ra2-CAR construct was co-transduced with mTa to produce mTA-IL13-CAR T cells. The mTa was constructed with murine CD28 hinge and murine CD28 transmembrane to avoid dimerization with CAR constructs which used a murine CD8 hinge and murine CD8 transmembrane domain.

Isolated T cells from C57BL/6 mouse spleen were stimulated with CD3/CD28 beads for co-transduction of the mTa and mIL13CAR viral supernatant. Cells were de-beaded 48 hours after transduction and stained for anti G4S3 and CD19-conjugated antibodies for the detection of mTa and CAR expression, respectively, using flow cytometry. As shown in FIG. 6, high transduction efficiency and stable co-expression was observed in murine T cells co-transduced with mTa and mIL13CAR.

### Example 7: mTa-mIL13 CAR T Cells Activate NFkB Signaling

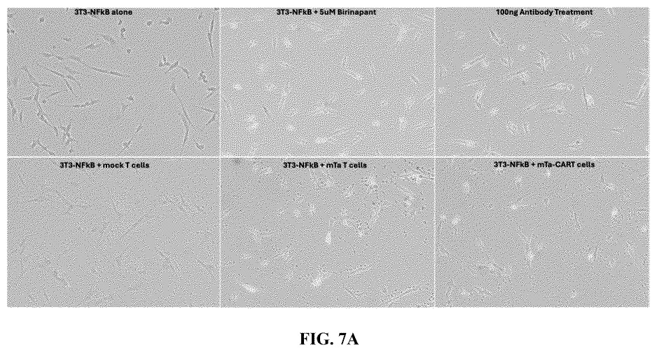

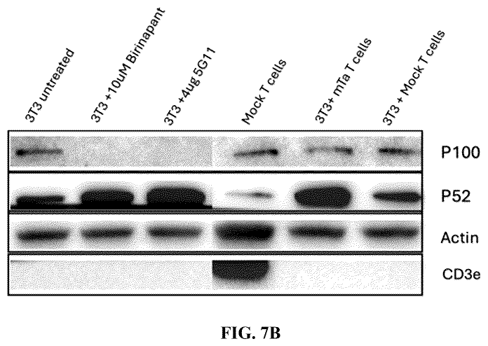

To determine whether mTa-mIL13 CAR T cells activate NFkB signaling, a NIH3T3 NFkB-GFP reporter cell line was generated to screen for mouse LTβR-agonistic activity. 1×10⁵ cells were plated in a 6 well plate and treated with either 5 μM of birinapant or 100 ng of 5G11, or co-cultured with mock T cells, co-cultured with mTa T cells, or co-cultured with mTa-IL13CAR T cells at a 1:1 ratio. Brightfield and GFP images were captured 24 hrs post co-culture using Nikon Eclipse Ti2-E motorized immunofluorescence microscope and merged using ImageJ. As shown in FIG. 7A, mTa-mIL13 CAR T cells activate NFkB signaling. Further, mTa functions like 5G11 in activating NFkB signaling. FIG. 7B shows a western blot of cell lysates demonstrating processing of p100 to p52, thereby confirming downstream LTβR activation.

### Example 8: CAR-T Cytotoxic Functions are Unaffected by mTa Expression

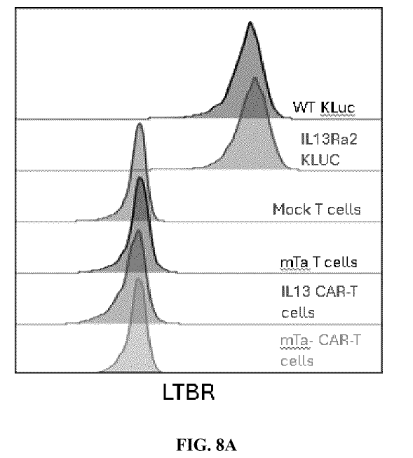

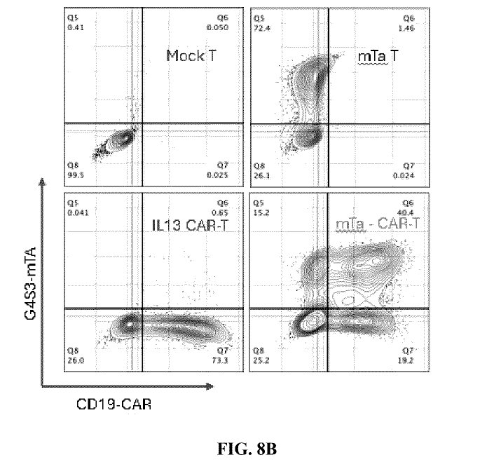

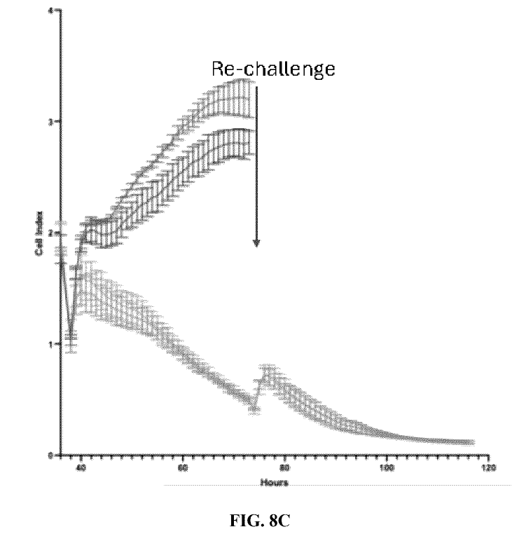

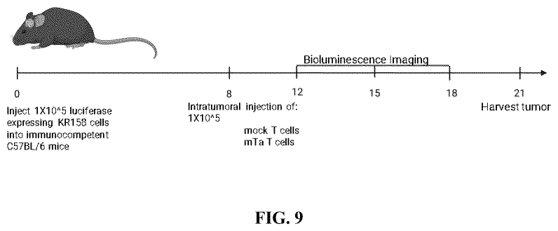

To confirm mTa do not impact CAR functions, an xCELLigence Killing Assay (Agilent) was performed using KR158 cells that express both LTβR and IL13Ra2. 1500 IL13Ra2-KLuc cells were plated in a 96 well xCELLigence plate and cell index was monitored for 36 hours before adding the effector cells at a 1:1 ratio near the 36 hour mark. To rechallenge the effector cells, an additional round of target cells was added 38 hours after the first round of killing. As shown in FIGS. 8A-C, CAR-T cytotoxic function is unaffected by mTa expression.

### Example 9: Assessing mTa T Cells In Vivo

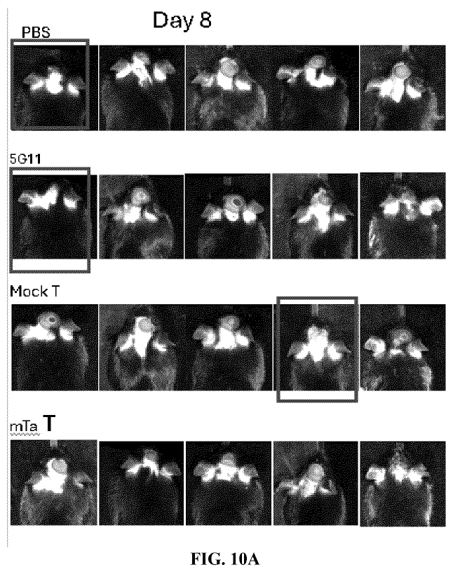

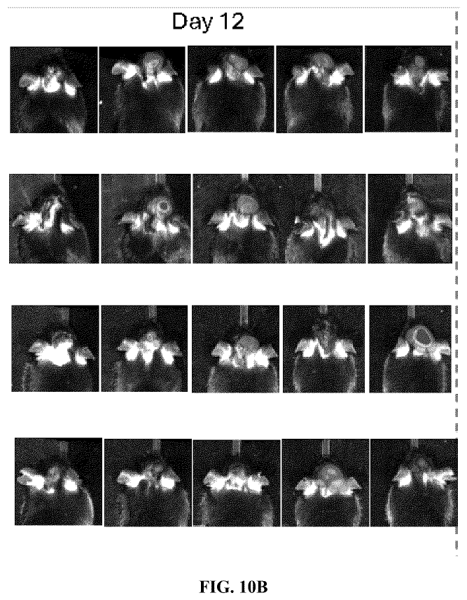

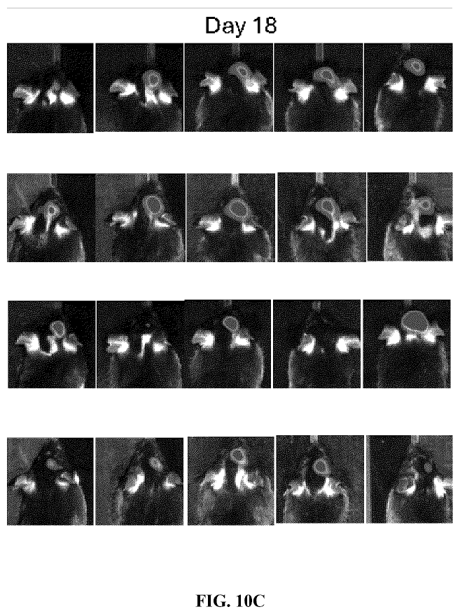

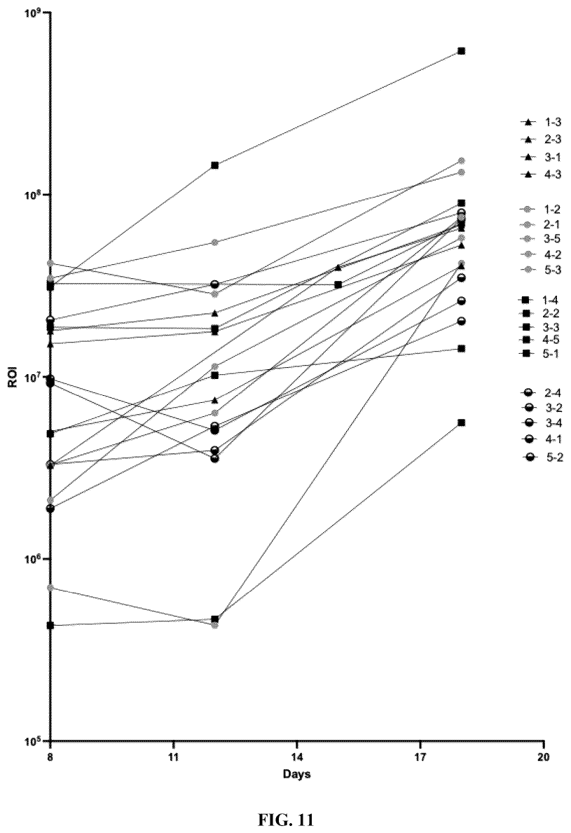

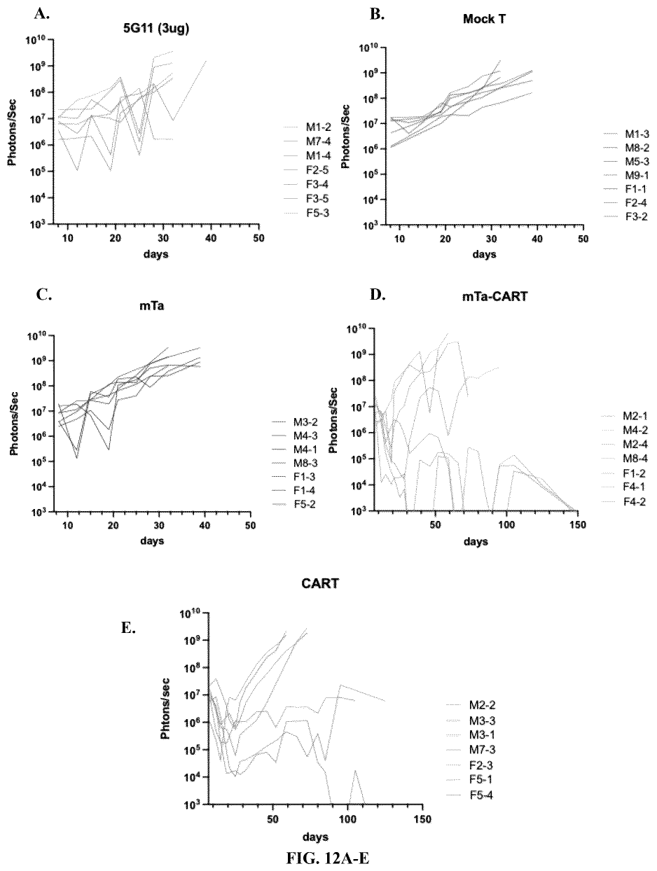

The experimental design used in the example is depicted in FIG. 9. Briefly, immunocompetent C57BL/6 mice were injected with 1×10⁵ KR158-luc cells. 8 days after KR158-luc injection, either 1×10⁵ mock T cells or 1×10⁵ mTa T cells were injected intratumorally. Bioluminescence imaging was performed at days 12-18 post-KR158-luc cell injection to assess tumor growth (FIGS. 10A-C). Mice were euthanized after 21 days, and brains were harvested after cardiac perfusion with PBS followed by fixation in 4% PFA. 200 Formalin-fixed, paraffin-embedded 10 μm axial brain sections were prepared for Hematoxylin and eosin (H&E) and immunohistochemistry (IHC) staining as described above in Example 2.

Between 8 and 18 days post-KR158-luc cell injection, mice treated with mTa T cells exhibited decreased tumor volume compared to 5G11, mock T cell, and PBS control treated mice (FIGS. 10A-C). As shown in FIG. 11, graphed regions of interest (ROIs) at day 18 confirm these data.

### Example 10: MTa Expression does not Impair CAR-T Therapeutic Efficacy

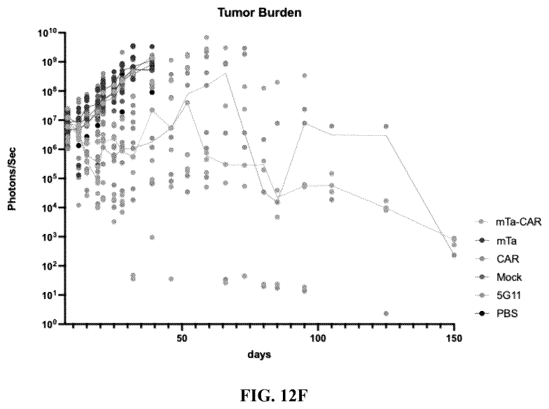

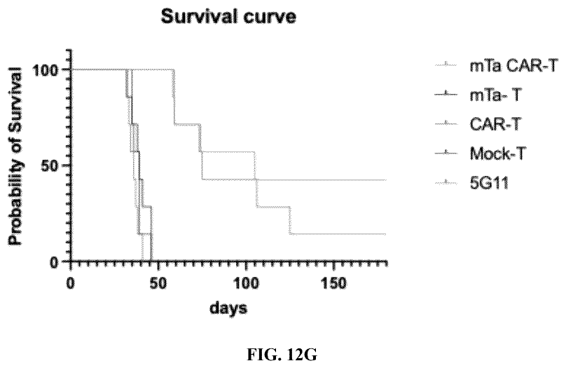

Bioluminescence imaging was performed to assess tumor burden and survival rate across different treatment groups. mTa cells or Mock T cells alone do not reduce tumor growth. However, CAR-T and mTa-CAR T both reduce tumor burden and improve survival, demonstrating that mTa does not negatively impact CAR efficiency in vivo (FIGS. 12A-G).

### Example 11: Repeated LTβR Agonist Administration In Vivo

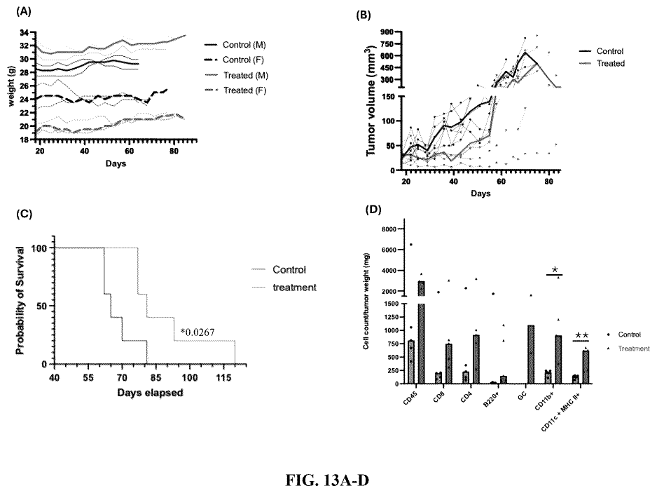

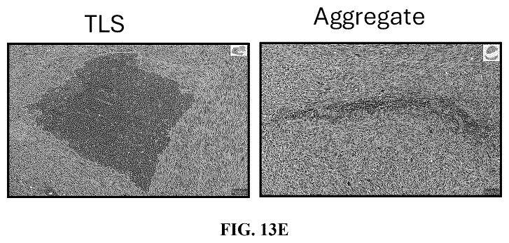

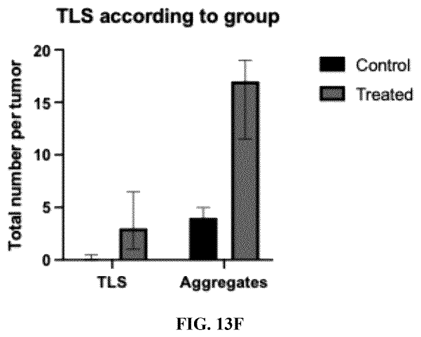

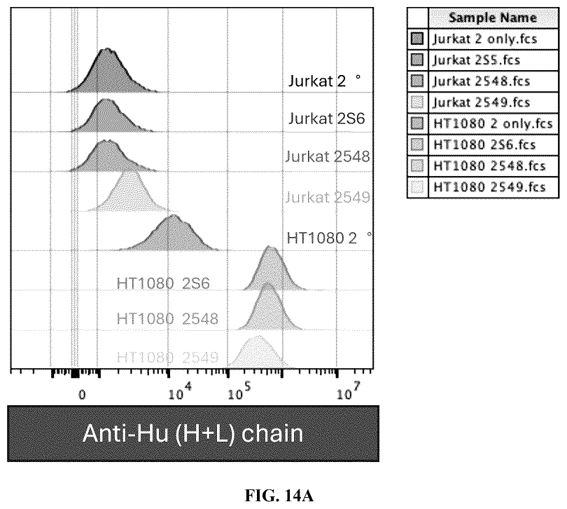

C57BL/6 mice were implanted subcutaneously with 5×10⁵ KR158 glioma cells. On day 18 post-implantation, mice were randomized (n=5 per group) to receive intratumoral injections of either the agonistic anti-LTβR antibody 5G11 (10 μg per dose) or PBS control, administered biweekly. Body weight and tumor volume were measured twice weekly (FIG. 13A-B). Tumor volume was calculated using the formula 0.5×(length× width²) (FIG. 13B). Mice were euthanized at humane endpoint, defined as development of ulceration and/or tumor length or width exceeding 15 mm. Tumors were harvested at endpoint for flow cytometric analysis of immune cell infiltration (FIG. 13C-D). As shown in FIGS. 13A-D, repeated intratumoral dosing of 5G11 in a subcutaneous KR158 model is safe, reduces tumor volume, and provides a survival benefit (n=5/group 5G11 vs PBS). Immune infiltration at humane endpoint is increased, and CD23⁺ germinal center B cells (indicative of functional TLS) were observed only in mice treated the longest. Histological analysis of tumor samples were performed. H&E slides show annotated TLS and lymphoid aggregates from sectioned tumors (FIG. 13E). Number of TLS and aggregates per tumor was quantified and graphed according to treatment group (FIG. 13F).

### Example 12: Human LTβR Antibody Generation and Validation

Three human LTβR antibodies, hLTβR-2S6 FcSTR, hLTβR-2S48 FcSTR, and hLTβR-2S49 FcSTR, were generated and tested for functional activity.

TABLE 4
Examples of Hu-LTBR antibody screening.

| | | | | Con- | | |
|---|---|---|---|---|---|---|
| | | | Amount | centration | Volume | |
| Name | Target | Description | (mg) | (mg/mL) | (mL) | Buffer |
| hLTBR-2S6 | hLTBR | Rabbit-human | 1 | 1 | 1 | PBS, |
| FcSTR | | chimeric anti-hLTBR- | | | | pH7.4 |
| | | 2S6 IgG, with Fc null | | | | |
| hLTBR- | hLTBR | Rabbit-human | 1.3 | 1.3 | 1 | PBS, |
| 2S48 | | chimeric anti-hLTBR- | | | | pH7.4 |
| FcSTR | | 2S48 IgG, with Fc null | | | | |
| hLTBR- | hLTBR | Rabbit-human | 0.3 | 1 | 0.3 | PBS, |
| 2S49 | | chimeric anti-hLTBR- | | | | pH7.4 |
| FcSTR | | 2S49 IgG, with Fc null | | | | |

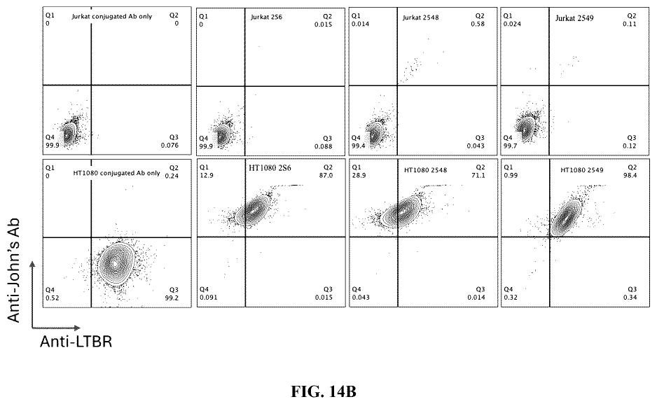

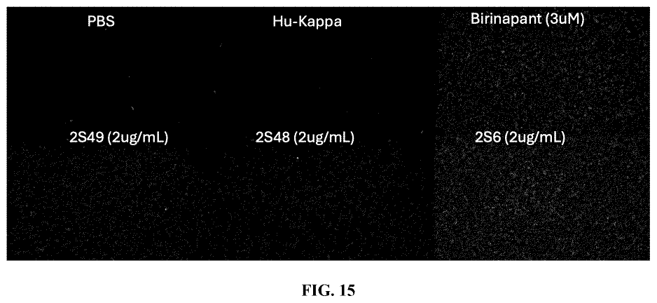

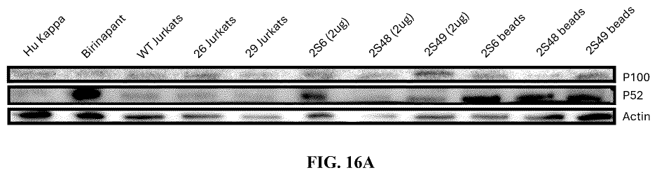

To confirm antibody binding to LTβR, HT1080 (LTβR⁺) and Jurkat (LTβR⁻) cells were incubated with primary antibodies against human LTβR (1:100 dilution, 1.5 hrs). Bound antibodies were detected with a secondary anti-human (H+L) conjugated antibody (1.5 hrs). A PE-conjugated anti-LTβR antibody was used as a positive control. Flow cytometry analysis confirmed target binding for all three antibodies, with clone 2549 displaying weaker binding compared to 256 and 2548 (FIGS. 14A-B).

To identify LTβR agonists that can activate NFkB signaling, an NF-κB reporter assay was used. HT1080 NF-κB reporter cells were seeded on day 0 and treated on day 1 with candidate anti-LTβR agonistic antibodies to assess receptor-mediated NF-κB activation. Birinapant was included as a positive control for NF-κB pathway activation, and human IgG kappa (Hu-Kappa) was used as an isotype control. Among the antibodies tested, clone 2S6 induced the highest GFP (FIG. 15).

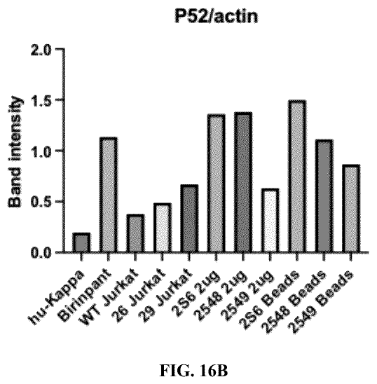

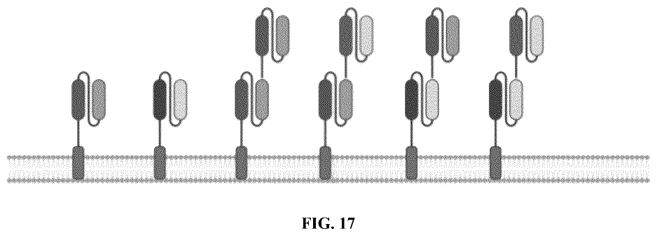

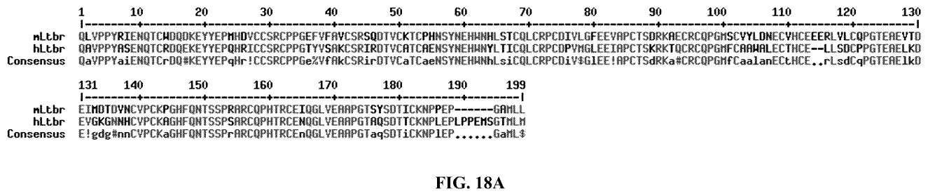

To assess the effects of antibody treatment on LTβR-mediated non-canonical NF-κB signaling, Western blot analysis was performed to detect the processing of the NF-κB precursor protein P100 to its active form, P52. HT1080 cells were plated on day 0 and treated after 24 hours with free antibody or antibody cross-linked to beads. Controls included untreated cells (negative control), Hu Kappa (negative/isotype control), and Birinapant (positive control for NFkB activation) (FIGS. 16A-B). The results demonstrated a dose-dependent and cross-linking-dependent processing of P100 to P52 following LTβR engagement. All tested antibodies, when cross-linked to beads, induced robust P100 to P52 conversion, indicating that receptor clustering significantly enhances LTβR agonist activity and further supports the utility of membrane-tethering strategies for optimizing LTβR activation. The Jurkat cells showed minimal processing of P100 to P52. In the soluble form, antibody hLTβR-2S6 FcSTR exhibited the strongest agonistic activity.

### Example 13: Production, Screening and Characterization of Mouse and Human LTβR

Sequence alignment comparing amino acid sequences of mLTβR and hLTβR was used for antibody generation (FIG. 18A). Size-exclusion chromatography was performed to confirm expression of mLTβR and hLTβR (FIG. 18B). Surface Plasmon Resonance (SPR) was used to assess the binding of hLTβR to commercial anti-hLTβR antibody (FIG. 18C). Western blot analysis was performed to confirm successful expression and purification of nine hLTβR Fab fragments (FIG. 18E). SPR was performed to select best candidates for further studies by assessing binding affinity and specificity of hLTβR Fab fragments (FIG. 18E-F). Thermal shift analysis was performed to determine thermal stability of LTβR 2S6 Fab, LTβR 2S48 Fab, and LTβR 2S49 Fab fragments (FIG. 18G).

INFORMAL SEQUENCE LISTING

LTBR Mouse Amino Acid Sequence
SEQ ID NO: 1
QLVPPYRIENQTCWDQDKEYYEPMHDVCCSRCPPGEFVFAVCSRSQDTVCKTCPHNSYN
EHWNHLSTCQLCRPCDIVLGFEEVAPCTSDRKAECRCQPGMSCVYLDNECVHCEEERLV
LCQPGTEAEVTDEIMDTDVNCVPCKPGHFQNTSSPRARCQPHTRCEIQGLVEAAPGTSYS
DTICKNPPEPGAMLL
LTBR Human Amino Acid Sequence
SEQ ID NO: 2
QAVPPYASENQTCRDQEKEYYEPQHRICCSRCPPGTYVSAKCSRIRDTVCATCAENSYNE
HWNYLTICQLCRPCDPVMGLEEIAPCTSKRKTQCRCQPGMFCAAWALECTHCELLSDCP
PGTEAELKDEVGKGNNHCVPCKAGHFQNTSSPSARCQPHTRCENQGLVEAAPGTAQSD
TTCKNPLEPLPPEMSGTMLM
Anti-hLTBR Heavy Chain Variable Domain (6)
SEQ ID NO: 3
QSVEESGGRLVTPGTPLTLTCTVSGFSLSNYAMSWVRQAPGKGLEWIGTITTGGSTYYAS
WAKGRFTISKTSSTTVDLKMTSLTIEDTATYFCARVDSGWRTGGIWGPGTLVTVSL
Anti-hLTBR Light Chain Variable Domain (6)
SEQ ID NO: 4
AYDMTQTPASVEVAVGGTVTINCQASQNIYNNLAWFQQKPGQPPKLLIYDTSDLASGVP
SRFKGSGSGTEYTLTISGVECADAATYYCQQGFTASGVDYTFGGGTEVVVK
Anti-hLTBR HC CDR1 (6)
SEQ ID NO: 5
NYAMS
Anti-hLTBR HC CDR2 (6)
SEQ ID NO: 6
TITTGGSTYYASWA
Anti-hLTBR HC CDR3 (6)
SEQ ID NO: 7
VDSGWRTGGI
Anti-hLTBR LC CDR1 (6)
SEQ ID NO: 8
QASQNIYNNLA
Anti-hLTBR LC CDR2 (6)
SEQ ID NO: 9
DTSDLAS
Anti-hLTBR LC CDR3 (6)
SEQ ID NO: 10
QQGFTASGVDYT
Anti-hLTBR Heavy Chain Variable Domain (8)
SEQ ID NO: 11
QSLEESGGDLVKPGGTLTLTCTASGFSFSNIYWICWVRQAPGKGLEWVACIDGGSSGTTY
YASWAKGRFTISKTSSTTVTLQMTSLTAADTATYFCAREFVDDANVYFDLWGPGTLVTV
SS
Anti-hLTBR Light Chain Variable Domain (8)
SEQ ID NO: 12
AQVLTQTASPVSAAVGGTVTINCQSSQSVASNYLSWYQQKSGQPPKLLIYSASTLASGVP
SRFKGSGSGTQFTLTISDLECDDAATYYCAGAYSDSSDIWAFGGGTELVVR
Anti-hLTBR HC CDR1 (8)
SEQ ID NO: 13
IYWIC
Anti-hLTBR HC CDR2 (8)
SEQ ID NO: 14
CIDGGSSGTTYYASWA
Anti-hLTBR HC CDR3 (8)
SEQ ID NO: 15
EFVDDANVYFDL
Anti-hLTBR LC CDR1 (8)
SEQ ID NO: 16
QSSQSVASNYLS
Anti-hLTBR LC CDR2 (8)
SEQ ID NO: 17
SASTLAS
Anti-hLTBR LC CDR3 (8)
SEQ ID NO: 18
AGAYSDSSDIWA
Anti-hLTBR Heavy Chain Variable Domain (20)
SEQ ID NO: 19
QSLEESGGDLVKPGASLTLTCTASGFSFSGYFFMCWVRQAPGKGLEWSACIYAGSSGNT
YSASWAKGRFTISKTSSTTVTLQMTSLTAADTATYFCARLDQNAAYVDLWGPGTLVTVS
S
Anti-hLTBR Light Chain Variable Domain (20)
SEQ ID NO: 20
AFALTQTPASVEAAVGGTVTIKCQASQTFSSYLSWYQQKPGQPPKLLIYRASTLASGVPS
RFSGSGFGTEFTLTISDLECADAATYFCQSYYNSINIDFTFGGGTEVVVR
Anti-hLTBR HC CDR1 (20)
SEQ ID NO: 21
YFFMC
Anti-hLTBR HC CDR2 (20)
SEQ ID NO: 22
CIYAGSSGNTYSASWA
Anti-hLTBR HC CDR3 (20)
SEQ ID NO: 23
LDQNAAYVDL
Anti-hLTBR LC CDR1 (20)
SEQ ID NO: 24
QASQTFSSYLS
Anti-hLTBR LC CDR2 (20)
SEQ ID NO: 25
RASTLAS
Anti-hLTBR LC CDR3 (20)
SEQ ID NO: 26
QSYYNSINIDFT
Anti-hLTBR Heavy Chain Variable Domain (48)
SEQ ID NO: 27
QSLEESGGDLVEPGASLTLTCTASGIDFSSYYYMCWVRQAPGKGLELSACIYDVSGSTW
YASGAKGRFTISKTSSTTVTLQMTSLTAADTATYFCARGYDSGSDYYYYGMDLWGPGTL
VTVSS
Anti-hLTBR Light Chain Variable Domain (48)
SEQ ID NO: 28
ALVMTQXASPVSAAVGDTVTINCQASQSIGSNLAWFQQKPGQPPKLLIYAASNLAFGVPS
RFSGSRFGTEYTLTISGVQCEDAATYYCLGTYSSSESAFGGGTELEIL
Anti-hLTBR HC CDR1 (48)
SEQ ID NO: 29
YYYMC
Anti-hLTBR HC CDR2 (48)
SEQ ID NO: 30
CIYDVSGSTWYASGA
Anti-hLTBR HC CDR3 (48)
SEQ ID NO: 31
GYDSGSDYYYYGMDL
Anti-hLTBR LC CDR1 (48)
SEQ ID NO: 32
QASQSIGSNLA
Anti-hLTBR LC CDR2 (48)
SEQ ID NO: 33
AASNLAF
Anti-hLTBR LC CDR3 (48)
SEQ ID NO: 34
LGTYSSSESA
Anti-hLTBR Heavy Chain Variable Domain (49)
SEQ ID NO: 35
QSLEESGGRLVTPGTPLTLTCTVSGFTISNYLMCWVRQAPGKGLEYIGIINTGSRTYYASW
AKGRFTISKTSTTVDLKITSPTTEDTATYFCARSYGSDIDAFAPWGPGTLVTVSS
Anti-hLTBR Light Chain Variable Domain (49)
SEQ ID NO: 36
AQVLTQTASSVXAAVGGTVTINCQASQSVYGNNYLSWYQQKPGQPPKLLIYQASVLAF
GVPSRFKGSGFGKQFTLTISGVQCEDAATYYCAGGYVCSSFDCSAFGGGTEVVVK
Anti-hLTBR HC CDR1 (49)
SEQ ID NO: 37
NYLMC
Anti-hLTBR HC CDR2 (49)
SEQ ID NO: 38
IINTGSRTYYASWA
Anti-hLTBR HC CDR3 (49)
SEQ ID NO: 39
SYGSDIDAFAP
Anti-hLTBR LC CDR1 (49)
SEQ ID NO: 40
QASQSVYGNNYLS
Anti-hLTBR LC CDR2 (49)
SEQ ID NO: 41
QASVLAF
Anti-hLTBR LC CDR3 (49)
SEQ ID NO: 42
AGGYVCSSFDCSA
Anti-hLTBR Heavy Chain Variable Domain (50)
SEQ ID NO: 43
QSLQESGGGLFQPGGSLALTCTSSGFSLTDNYVMCWVRQAPGKGLEWIACIITGSSGGTY
YASWAKGRFTISKTSSTTLTLQMTSLTAADTATYFCARSGWELFNLWGPGTLVTVSS
Anti-hLTBR Light Chain Variable Domain (50)
SEQ ID NO: 44
AIVMTQTPSSTSAAVQGTVTISCQASESLISNYLSWYQQKPGQPPKLLIYDASTLASGVPS
RFKGSGSGTQFTLTITNVQREDAATYYCAGYKTSSDDGWAFGAGTNVEIK
Anti-hLTBR HC CDR1 (50)
SEQ ID NO: 45
NYVMC
Anti-hLTBR HC CDR2 (50)
SEQ ID NO: 46
CIITGSSGGTYYASWA
Anti-hLTBR HC CDR3 (50)
SEQ ID NO: 47
SGWELFNL
Anti-hLTBR LC CDR1 (50)
SEQ ID NO: 48
QASESLISNYLS
Anti-hLTBR LC CDR2 (50)
SEQ ID NO: 49
DASTLAS
Anti-hLTBR LC CDR3 (50)
SEQ ID NO: 50
AGYKTSSDDGWA
Anti-hLTBR Heavy Chain Variable Domain (51)
SEQ ID NO: 51
QSVEESGGRLVTPGTPLTLTCTASGFSLSGYYLSWLRQAPGKGLEWIGSIWNDNTYYAN
WARGRFTISKTSTTVDLKITSPTTEDTATYFCARDAAYGGVGGAFDPWGPGTLVTVSS
Anti-hLTBR Light Chain Variable Domain (51)
SEQ ID NO: 52
AAVMTQTPSSVSAAVGGTITINCQSSPSVYSNYLSWFQQKPGQPPKLLIYGASTLASGVPS
RFKGSGSGLQFTLSISDVQCDDAATYYCAGGYSGTSDTFAFGGGTEVVVE
Anti-hLTBR HC CDR1 (51)
SEQ ID NO: 53
GYYLS
Anti-hLTBR HC CDR2 (51)
SEQ ID NO: 54
SIWNDNTYYANWA
Anti-hLTBR HC CDR3 (51)
SEQ ID NO: 55
DAAYGGVGGAFDP
Anti-hLTBR LC CDR1 (51)
SEQ ID NO: 56
QSSPSVYSNYLS
Anti-hLTBR LC CDR2 (51)
SEQ ID NO: 57
GASTLAS
Anti-hLTBR LC CDR3 (51)
SEQ ID NO: 58
AGGYSGTSDTFA
Anti-hLTBR Heavy Chain Variable Domain (52)
SEQ ID NO: 59
QSLEESGGDLVKPGASLTLTCTASGFDFSSVAMCWVRQAPGKGLEWIGCIYPISATTYYPS
WAKGRFTISKTSSTTVTLQMTSLTAADTATYFCATYNGAPYYYINLWGPGTLVTVSS
Anti-hLTBR Light Chain Variable Domain (52)
SEQ ID NO: 60
DVVMPQTPSSTSTAVGGTVTIKCQASQNIGNYLAWYQQKPGQPPKLLIYYAATLASGVPS
RFKGGRSGTQFTLTISDVQREDAATYYCLGGTASGSSRTAFGGGTELEIL
Anti-hLTBR HC CDR1 (52)
SEQ ID NO: 61
SVAMC
Anti-hLTBR HC CDR2 (52)
SEQ ID NO: 62
CIYPISATTYYPSWA
Anti-hLTBR HC CDR3 (52)
SEQ ID NO: 63
YNGAPYYYINL
Anti-hLTBR LC CDR1 (52)
SEQ ID NO: 64
QASQNIGNYLA
Anti-hLTBR LC CDR2 (52)
SEQ ID NO: 65
YAATLAS
Anti-hLTBR LC CDR3 (52)
SEQ ID NO: 66
LGGTASGSSRTA
Anti-hLTBR Heavy Chain Variable Domain (53)
SEQ ID NO: 67
QEQVVESGGDLVQPAGSLTLTCTAXGFSLSSAYWICWVRQAPGKGLEWIACIYTGTSGSS
DYASWAKGRFTISKTSSTTVTLQMTSLTAADTATYFCARGLLGYGDFGYVFKLWGPGTL
VTVSS
Anti-hLTBR Light Chain Variable Domain (53)
SEQ ID NO: 68
ADIVMTQTPASVEAAVGGTVTIKCQASQNIYSNLLAWYQQKPGQPPKLLIYRASTLEFGV
PSRFKGSGFGTEFTLTISDLECADAATYYCQSYYYYSGSSVNDFGGGTEVVVK
Anti-hLTBR HC CDR1 (53)
SEQ ID NO: 69
AYWIC
Anti-hLTBR HC CDR2 (53)
SEQ ID NO: 70
CIYTGTSGSSDYASWAK
Anti-hLTBR HC CDR3 (53)
SEQ ID NO: 71
GLLGYGDFGYVFKL
Anti-hLTBR LC CDR1 (53)
SEQ ID NO: 72
QASQNIYSNLLA
Anti-hLTBR LC CDR2 (53)
SEQ ID NO: 73
RASTLEF
Anti-hLTBR LC CDR3 (53)
SEQ ID NO: 74
QSYYYYSGSSVND
Anti-hLTBR Heavy Chain Variable Domain (54)
SEQ ID NO: 75
QSXEESGGRLVTPGGSLTLTCTVSGIDLNADHMFWVRQSPKKGLEWIGIIGNNGNTYYAS
WAKGRFTISKTSTTVSLQVSSPTTEDTATYFCARPIQWLAGGYYFNMWGPGTLVTVSL
Anti-hLTBR Light Chain Variable Domain (54)
SEQ ID NO: 76
AIEMTQFPPSLFAFVGDTVTINCLGSENVYSAVAWYQQKPGKPPTLLIYGASNLEFGVPPR
FSGSGFGTDYTLTIGGVQAEDAATYVCQGYSNTPTFGAGTKVEIK
Anti-hLTBR HC CDR1 (54)
SEQ ID NO: 77
ADHMF
Anti-hLTBR HC CDR2 (54)
SEQ ID NO: 78
IIGNNGNTYYASWA
Anti-hLTBR HC CDR3 (54)
SEQ ID NO: 79
PIQWLAGGYYFNM
Anti-hLTBR LC CDR1 (54)
SEQ ID NO: 80
LGSENVYSAVA
Anti-hLTBR LC CDR2 (54)
SEQ ID NO: 81
GASNLEF
Anti-hLTBR LC CDR3 (54)
SEQ ID NO: 82
QGYSNTPT
Anti-hLTBR Heavy Chain Variable Domain (55)
SEQ ID NO: 83
QSVEESGGRLVTPGGSLTLTCTVSGFSLSNYGMGWVRQAPGKGLEYIGIINAYNTIYYAS
WAKGRFTISKTSTTVTLKMTSLTAADTATYFCARNLGWSSINVWGPGTLVTVSL
Anti-hLTBR Light Chain Variable Domain (55)
SEQ ID NO: 84
AVVMTQTPSSTSAVVGGTVTIKCQASRSIGSSLSWYQQKPGQPPKLLIYETSELTSGVPSR
FSGSGSGTDYTLTISGVQCDDAATYYCLGYFGNTDDGAAFGGGTDLEILC
Anti-hLTBR HC CDR1 (55)
SEQ ID NO: 85
NYGMG
Anti-hLTBR HC CDR2 (55)
SEQ ID NO: 86
IINAYNTIYYASWA
Anti-hLTBR HC CDR3 (55)
SEQ ID NO: 87
NLGWSSINV
Anti-hLTBR LC CDR1 (55)
SEQ ID NO: 88
QASRSIGSSLS
Anti-hLTBR LC CDR2 (55)
SEQ ID NO: 89
ETSELTS
Anti-hLTBR LC CDR3 (55)
SEQ ID NO: 90
LGYFGNTDDGAA
Anti-hLTBR Heavy Chain Variable Domain (57)
SEQ ID NO: 91
QSXEESGGRLVTPGTPLTLTCTAFGFSLSGYYLSWVRQAPGKGLEWIGSIWNDNIYYATW
ARGRFTISKTSTTVDLKITSPTTEDTATYFCARDAAYGGYGGAFDPWGPGTLVTVSS
Anti-hLTBR Light Chain Variable Domain (57)
SEQ ID NO: 92
AAVMTQTPSSVSAAVGGTVTINCQSSPSVYNNYLSWFQQKPGQPPKLLIYGASTLASGVP
SRFKGSGSGTQFTLTISDLECDDAATYYCAGGYSGTSDTFAFGGGTEVVVK
Anti-hLTBR HC CDR1 (57)
SEQ ID NO: 53
GYYLS
Anti-hLTBR HC CDR2 (57)
SEQ ID NO: 93
SIWNDNIYYATWA
Anti-hLTBR HC CDR3 (57)
SEQ ID NO: 94
DAAYGGYGGAFDP
Anti-hLTBR LC CDR1 (57)
SEQ ID NO: 95
QSSPSVYNNYLS
Anti-hLTBR LC CDR2 (57)
SEQ ID NO: 57
GASTLAS
Anti-hLTBR LC CDR3 (57)
SEQ ID NO: 58
AGGYSGTSDTFA
Anti-hLTBR Heavy Chain Variable Domain (58)
SEQ ID NO: 96
QSXEESGGRLVTPGTPLTLTCTAFGFSLSTYAMTWVRQAPGKGLEWIGIIYASGSVVYYAT
WARGRFTISKTSTTVDLKITSPTTEDTATYFCARGGAGTNSWNLGIWGPGTLVTVSL
Anti-hLTBR Light Chain Variable Domain (58)
SEQ ID NO: 97
ALVMTQTPSSTSTAVGDTVTIKCQASQSIDSVYLSWYQQKPGQRPKLLIYEASTLAFGVP
SRFKGSGSGKQFTLTISGVQRDDAATYYCLGSYSSNDSAFGGGTELEIL
Anti-hLTBR HC CDR1 (58)
SEQ ID NO: 98
TYAMT
Anti-hLTBR HC CDR2 (58)
SEQ ID NO: 99
IIYASGSVVYYATWA
Anti-hLTBR HC CDR3 (58)
SEQ ID NO: 100
GGAGTNSWNLGI
Anti-hLTBR LC CDR1 (58)
SEQ ID NO: 101
QASQSIDSVYLS
Anti-hLTBR LC CDR2 (58)
SEQ ID NO: 102
EASTLAF
Anti-hLTBR LC CDR3 (58)
SEQ ID NO: 103
LGSYSSNDSA
Anti-hLTBR Heavy Chain Variable Domain (59)
SEQ ID NO: 104
QSXEESGGRLVTPGTPLTLTCTAFGFSLSGYYLSWLRQAPGKGLEWIGSIWNDNTYYAN
WARGRFTISKTSTTVDLKITSPTTEDTATYFCARDAAYGGVGGAFDPWGPGTLVTVSS
Anti-hLTBR Light Chain Variable Domain (59)
SEQ ID NO: 105
AAVMTQTPSSVFAAVGGTITINCQSSPSVYSNYLSWFQQKPGQPPKLLIYGASTLASGVPS
RFKGSGSGLQFTLSISDVQCDDAATYYCAGGYSGTSDTFAFGGGTEVVVE
Anti-hLTBR HC CDR1 (59)
SEQ ID NO: 53
GYYLS
Anti-hLTBR HC CDR2 (59)
SEQ ID NO: 54
SIWNDNTYYANWA
Anti-hLTBR HC CDR3 (59)
SEQ ID NO: 55
DAAYGGVGGAFDP
Anti-hLTBR LC CDR1 (59)
SEQ ID NO: 56
QSSPSVYSNYLS
Anti-hLTBR LC CDR2 (59)
SEQ ID NO: 57
GASTLAS
Anti-hLTBR LC CDR3 (59)
SEQ ID NO: 58
AGGYSGTSDTFA
Anti-hLTBR Heavy Chain Variable Domain (60)
SEQ ID NO: 106
QSVEESGGRLVTPGTPLTLTCTASGFSLNDYDWNWVRQAPGEELKWVATIHTYHTTYYA
NWAKGRFTISRTSTTVDLKMTSPTTEDTATYFCGANNVWGPGTLVTVSL
Anti-hLTBR Light Chain Variable Domain (60)
SEQ ID NO: 107
AQVXTQTPSXVXAXVGGTVTINCQASQSVYKNNXLSWFQQKPGQPPKRLIYYVSTLNF
GVSSRFKGSGSGTQFTLTISDVRCDDAATYYCLGNYDCRSADCLVFGGGTEVLVK
Anti-hLTBR HC CDR1 (60)
SEQ ID NO: 108
DYDWN
Anti-hLTBR HC CDR2 (60)
SEQ ID NO: 109
TIHTYHTTYYANWA
Anti-hLTBR HC CDR3 (60)
SEQ ID NO: 110
NNV
Anti-hLTBR LC CDR1 (60)
SEQ ID NO: 111
TBD
Anti-hLTBR LC CDR2 (60)
SEQ ID NO: 112
YVSTLN
Anti-hLTBR LC CDR3 (60)
SEQ ID NO: 113
LGNYDCRSADCLV
Anti-hLTBR Heavy Chain Variable Domain (61)
SEQ ID NO: 114
QSVEESGGRLVTPGTPLTLTCTVFGFSLSNYDMSWVRQAPGKGLEWIGIISTGDIKYYAN
WARGRFTISKTSTTVDLQITSLTTEDTATYFCARESHWGIENVWGPGTLVTVSL
Anti-hLTBR Light Chain Variable Domain (61)
SEQ ID NO: 115
AQVLTQTPSSVSAAVGGTVTINCQSSPSVYRNNLLSWFQQKSGQPPKLLIYQVSELAFGV
PSRFSGSGSGTEFTLTISDVQCDDGATYYCAGGYSRNSDRRAFGGGTEVVV
Anti-hLTBR HC CDR1 (61)
SEQ ID NO: 116
NYDMS
Anti-hLTBR HC CDR2 (61)
SEQ ID NO: 117
IISTGDIKYYANWA
Anti-hLTBR HC CDR3 (61)
SEQ ID NO: 118
ESHWGIENV
Anti-hLTBR LC CDR1 (61)
SEQ ID NO: 119
QSSPSVYRNNLLS
Anti-hLTBR LC CDR2 (61)
SEQ ID NO: 120
QVSELAF
Anti-hLTBR LC CDR3 (61)
SEQ ID NO: 121
GGYSRNSDRRA
Rabbit-Human chimeric sequences for anti-hLTBR Abs with Fc silent mutation
Anti-hLTBR Heavy Chain (6)
SEQ ID NO: 122
QSVEESGGRLVTPGTPLTLTCTVSGFSLSNYAMSWVRQAPGKGLEWIGTITTGGSTYYAS
WAKGRFTISKTSSTTVDLKMTSLTIEDTATYFCARVDSGWRTGGIWGPGTLVTVSLASTK
GPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPESTRGPSVFLF
PPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQ
GNVFSCSVMHEALHNHYTQKSLSLSPGK
Anti-hLTBR Light Chain (6)
SEQ ID NO: 123
AYDMTQTPASVEVAVGGTVTINCQASQNIYNNLAWFQQKPGQPPKLLIYDTSDLASGVP
SRFKGSGSGTEYTLTISGVECADAATYYCQQGFTASGVDYTFGGGTEVVVKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Anti-hLTBR Heavy Chain (8)
SEQ ID NO: 124
QSLEESGGDLVKPGGTLTLTCTASGFSFSNIYWICWVRQAPGKGLEWVACIDGGSSGTTY
YASWAKGRFTISKTSSTTVTLQMTSLTAADTATYFCAREFVDDANVYFDLWGPGTLVTV
SSASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPESTR
GPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDK
SRWQQGNVFSCSVMHEALHNHYTQKSLSLSPGK
Anti-hLTBR Light Chain (8)
SEQ ID NO: 125
AQVLTQTASPVSAAVGGTVTINCQSSQSVASNYLSWYQQKSGQPPKLLIYSASTLASGVP
SRFKGSGSGTQFTLTISDLECDDAATYYCAGAYSDSSDIWAFGGGTELVVRRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Anti-hLTBR Heavy Chain (20)
SEQ ID NO: 126
QSLEESGGDLVKPGASLTLTCTASGFSFSGYFFMCWVRQAPGKGLEWSACIYAGSSGNT
YSASWAKGRFTISKTSSTTVTLQMTSLTAADTATYFCARLDQNAAYVDLWGPGTLVTVS
SASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPESTRG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKS
RWQQGNVFSCSVMHEALHNHYTQKSLSLSPGK
Anti-hLTBR Light Chain (20)
SEQ ID NO: 127
AFALTQTPASVEAAVGGTVTIKCQASQTFSSYLSWYQQKPGQPPKLLIYRASTLASGVPS
RFSGSGFGTEFTLTISDLECADAATYFCQSYYNSINIDFTFGGGTEVVVRRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC
Anti-hLTBR Heavy Chain (48)
SEQ ID NO: 128
QSLEESGGDLVEPGASLTLTCTASGIDFSSYYYMCWVRQAPGKGLELSACIYDVSGSTW
YASGAKGRFTISKTSSTTVTLQMTSLTAADTATYFCARGYDSGSDYYYYGMDLWGPGTL
VTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAP
ESTRGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPGK
Anti-hLTBR Light Chain (48)
SEQ ID NO: 129
ALVMTQXASPVSAAVGDTVTINCQASQSIGSNLAWFQQKPGQPPKLLIYAASNLAFGVPS
RFSGSRFGTEYTLTISGVQCEDAATYYCLGTYSSSESAFGGGTELEILRTVAAPSVFIFPPS
DEQLKSGTASVVCLLNNFYPREAKVQWKVDNALQSGNSQESVTEQDSKDSTYSLSSTLT
LSKADYEKHKVYACEVTHQGLSSPVTKSFNRGEC
Anti-hLTBR Heavy Chain (49)
SEQ ID NO: 130
QSLEESGGRLVTPGTPLTLTCTVSGFTISNYLMCWVRQAPGKGLEYIGIINTGSRTYYASW
AKGRFTISKTSTTVDLKITSPTTEDTATYFCARSYGSDIDAFAPWGPGTLVTVSSASTKGPS
VFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPESTRGPSVFLFPPK
PKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVF
SCSVMHEALHNHYTQKSLSLSPGK
Anti-hLTBR Light Chain (49)
SEQ ID NO: 131
AQVLTQTASSVXAAVGGTVTINCQASQSVYGNNYLSWYQQKPGQPPKLLIYQASVLAF
GVPSRFKGSGFGKQFTLTISGVQCEDAATYYCAGGYVCSSFDCSAFGGGTEVVVKRTVA
APSVFIFPPSDEQLKSGTASVVCLLNNFYPREAKVQWKVDNALQSGNSQESVTEQDSKD
STYSLSSTLTLSKADYEKHKVYACEVTHQGLSSPVTKSFNRGEC
Anti-hLTBR Heavy Chain (50)
SEQ ID NO: 132
QSLQESGGGLFQPGGSLALTCTSSGFSLTDNYVMCWVRQAPGKGLEWIACIITGSSGGTY
YASWAKGRFTISKTSSTTLTLQMTSLTAADTATYFCARSGWELFNLWGPGTLVTVSSAST
KGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPESTRGPSVFL
FPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQ
GNVFSCSVMHEALHNHYTQKSLSLSPGK
Anti-hLTBR Light Chain (50)
SEQ ID NO: 133
AIVMTQTPSSTSAAVQGTVTISCQASESLISNYLSWYQQKPGQPPKLLIYDASTLASGVPS
RFKGSGSGTQFTLTITNVQREDAATYYCAGYKTSSDDGWAFGAGTNVEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Anti-hLTBR Heavy Chain (51)
SEQ ID NO: 134
QSVEESGGRLVTPGTPLTLTCTASGFSLSGYYLSWLRQAPGKGLEWIGSIWNDNTYYAN
WARGRFTISKTSTTVDLKITSPTTEDTATYFCARDAAYGGVGGAFDPWGPGTLVTVSSAS
TKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPESTRGPS
VFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSR
WQQGNVFSCSVMHEALHNHYTQKSLSLSPGK
Anti-hLTBR Light Chain (51)
SEQ ID NO: 135
AAVMTQTPSSVSAAVGGTITINCQSSPSVYSNYLSWFQQKPGQPPKLLIYGASTLASGVPS
RFKGSGSGLQFTLSISDVQCDDAATYYCAGGYSGTSDTFAFGGGTEVVVERTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Anti-hLTBR Heavy Chain (52)
SEQ ID NO: 136
QSLEESGGDLVKPGASLTLTCTASGFDFSSVAMCWVRQAPGKGLEWIGCIYPISATTYYPS
WAKGRFTISKTSSTTVTLQMTSLTAADTATYFCATYNGAPYYYINLWGPGTLVTVSSAST
KGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPESTRGPSVFL
FPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQ
GNVFSCSVMHEALHNHYTQKSLSLSPGK
Anti-hLTBR Light Chain (52)
SEQ ID NO: 137
DVVMPQTPSSTSTAVGGTVTIKCQASQNIGNYLAWYQQKPGQPPKLLIYYAATLASGVPS
RFKGGRSGTQFTLTISDVQREDAATYYCLGGTASGSSRTAFGGGTELEILRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC
Anti-hLTBR Heavy Chain (53)
SEQ ID NO: 138
QEQVVESGGDLVQPAGSLTLTCTAXGFSLSSAYWICWVRQAPGKGLEWIACIYTGTSGSS
DYASWAKGRFTISKTSSTTVTLQMTSLTAADTATYFCARGLLGYGDFGYVFKLWGPGTL
VTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAP
ESTRGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPGK
Anti-hLTBR Light Chain (53)
SEQ ID NO: 139
ADIVMTQTPASVEAAVGGTVTIKCQASQNIYSNLLAWYQQKPGQPPKLLIYRASTLEFGV
PSRFKGSGFGTEFTLTISDLECADAATYYCQSYYYYSGSSVNDFGGGTEVVVKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
Anti-hLTBR Heavy Chain (54)
SEQ ID NO: 140
QSXEESGGRLVTPGGSLTLTCTVSGIDLNADHMFWVRQSPKKGLEWIGIIGNNGNTYYAS
WAKGRFTISKTSTTVSLQVSSPTTEDTATYFCARPIQWLAGGYYFNMWGPGTLVTVSLAS
TKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPESTRGPS
VFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSR
WQQGNVFSCSVMHEALHNHYTQKSLSLSPGK
Anti-hLTBR Light Chain (54)
SEQ ID NO: 141
AIEMTQFPPSLFAFVGDTVTINCLGSENVYSAVAWYQQKPGKPPTLLIYGASNLEFGVPPR
FSGSGFGTDYTLTIGGVQAEDAATYVCQGYSNTPTFGAGTKVEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
Anti-hLTBR Heavy Chain (55)
SEQ ID NO: 142
QSVEESGGRLVTPGGSLTLTCTVSGFSLSNYGMGWVRQAPGKGLEYIGIINAYNTIYYAS
WAKGRFTISKTSTTVTLKMTSLTAADTATYFCARNLGWSSINVWGPGTLVTVSLASTKGP
SVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPESTRGPSVFLFPP
KPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGN
VFSCSVMHEALHNHYTQKSLSLSPGK
Anti-hLTBR Light Chain (55)
SEQ ID NO: 143
AVVMTQTPSSTSAVVGGTVTIKCQASRSIGSSLSWYQQKPGQPPKLLIYETSELTSGVPSR
FSGSGSGTDYTLTISGVQCDDAATYYCLGYFGNTDDGAAFGGGTDLEILCRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Anti-hLTBR Heavy Chain (57)
SEQ ID NO: 144
QSXEESGGRLVTPGTPLTLTCTAFGFSLSGYYLSWVRQAPGKGLEWIGSIWNDNIYYATW
ARGRFTISKTSTTVDLKITSPTTEDTATYFCARDAAYGGYGGAFDPWGPGTLVTVSSAST
KGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPESTRGPSVFL
FPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQ
GNVFSCSVMHEALHNHYTQKSLSLSPGK
Anti-hLTBR Light Chain (57)
SEQ ID NO: 145
AAVMTQTPSSVSAAVGGTVTINCQSSPSVYNNYLSWFQQKPGQPPKLLIYGASTLASGVP
SRFKGSGSGTQFTLTISDLECDDAATYYCAGGYSGTSDTFAFGGGTEVVVKRTVAAPSVF
IFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
Anti-hLTBR Heavy Chain (58)
SEQ ID NO: 146
QSXEESGGRLVTPGTPLTLTCTAFGFSLSTYAMTWVRQAPGKGLEWIGIIYASGSVVYYAT
WARGRFTISKTSTTVDLKITSPTTEDTATYFCARGGAGTNSWNLGIWGPGTLVTVSLAST
KGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPESTRGPSVFL
FPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQ
GNVFSCSVMHEALHNHYTQKSLSLSPGK
Anti-hLTBR Light Chain (58)
SEQ ID NO: 147
ALVMTQTPSSTSTAVGDTVTIKCQASQSIDSVYLSWYQQKPGQRPKLLIYEASTLAFGVP
SRFKGSGSGKQFTLTISGVQRDDAATYYCLGSYSSNDSAFGGGTELEILRTVAAPSVFIFPP
SDEQLKSGTASVVCLLNNFYPREAKVQWKVDNALQSGNSQESVTEQDSKDSTYSLSSTL
TLSKADYEKHKVYACEVTHQGLSSPVTKSFNRGEC
Anti-hLTBR Heavy Chain (59)
SEQ ID NO: 148
QSXEESGGRLVTPGTPLTLTCTAFGFSLSGYYLSWLRQAPGKGLEWIGSIWNDNTYYAN
WARGRFTISKTSTTVDLKITSPTTEDTATYFCARDAAYGGVGGAFDPWGPGTLVTVSSAS
TKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPESTRGPS
VFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSR
WQQGNVFSCSVMHEALHNHYTQKSLSLSPGK
Anti-hLTBR Light Chain (59)
SEQ ID NO: 149
AAVMTQTPSSVFAAVGGTITINCQSSPSVYSNYLSWFQQKPGQPPKLLIYGASTLASGVPS
RFKGSGSGLQFTLSISDVQCDDAATYYCAGGYSGTSDTFAFGGGTEVVVERTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Anti-hLTBR Heavy Chain (60)
SEQ ID NO: 150
QSVEESGGRLVTPGTPLTLTCTASGFSLNDYDWNWVRQAPGEELKWVATIHTYHTTYYA
NWAKGRFTISRTSTTVDLKMTSPTTEDTATYFCGANNVWGPGTLVTVSLASTKGPSVFPL
APSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPESTRGPSVFLFPPKPKD
TLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCS
VMHEALHNHYTQKSLSLSPGK
Anti-hLTBR Light Chain (60)
SEQ ID NO: 151
AQVXTQTPSXVXAXVGGTVTINCQASQSVYKNNXLSWFQQKPGQPPKRLIYYVSTLNF
GVSSRFKGSGSGTQFTLTISDVRCDDAATYYCLGNYDCRSADCLVFGGGTEVLVKRTVA
APSVFIFPPSDEQLKSGTASVVCLLNNFYPREAKVQWKVDNALQSGNSQESVTEQDSKD
STYSLSSTLTLSKADYEKHKVYACEVTHQGLSSPVTKSFNRGEC
Anti-hLTBR Heavy Chain (61)
SEQ ID NO: 152
QSVEESGGRLVTPGTPLTLTCTVFGFSLSNYDMSWVRQAPGKGLEWIGIISTGDIKYYAN
WARGRFTISKTSTTVDLQITSLTTEDTATYFCARESHWGIENVWGPGTLVTVSLASTKGPS
VFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPESTRGPSVFLFPPK
PKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVF
SCSVMHEALHNHYTQKSLSLSPGK
Anti-hLTBR Light Chain (61)
SEQ ID NO: 153
AQVLTQTPSSVSAAVGGTVTINCQSSPSVYRNNLLSWFQQKSGQPPKLLIYQVSELAFGV
PSRFSGSGSGTEFTLTISDVQCDDGATYYCAGGYSRNSDRRAFGGGTEVVVRTVAAPSVF
IFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
Anti-hLTBR Light Chain (61)
SEQ ID NO: 153
AQVLTQTPSSVSAAVGGTVTINCQSSPSVYRNNLLSWFQQKSGQPPKLLIYQVSELAFGV
PSRFSGSGSGTEFTLTISDVQCDDGATYYCAGGYSRNSDRRAFGGGTEVVVRTVAAPSVF
IFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
m Ta sequence (GMCSF ss+ LC + (G4S)3 + HC + G3 + CD28h + CD28tm + CD28tcy)
SEQ ID NO: 154
ATGCTGCTGCTGGTGACCAGCCTGCTGCTGTGCGAGCTGCCACATCCTGCCTTCCTGC
TGATCCCTGATGTGCAGATGACCCAGAGCCCCAGCTACCTGGCCGCCAGCCCCGGCG
AATCTGTGTCAATTAGGTGCAAGGCCAGCAAGAGCATCTCCAACAACCTGGCCTGGT
ACCAGGAAAAACCCGGCAAGGCCAACAAGCTGCTGATCCATAGCGGCTCTACTCTGC
AGTCCGGGACCCCAAGCCGCTTCAGTGGGTCCGGCAGTGGAACAGATTTCACCCTGA
CCATCCGGAGCCTGGAGTTTGAGGACTTTGCCGTGTATTACTGCCAGCAGTACAACG
AATACCCTTACACTTTCGGCGCCGGCACCAAGCTGGAGCTGAAAAGAGCTGACGCCG
GCGGCGGGGGAAGTGGAGGCGGAGGCAGCGGCGGCGGCGGTTCCCAGGTGCAGCT
GAAGGAGTCAGGACCAGGCCTGGTGCAACCAAGCCAGACACTGTCCCTGACATGTA
CAGTGTCCGGCTTCAGCCTGACCACATACTCTGTGCACTGGGTGAGACAGCACAGCG
GCAAATCACTGGAGTGGATGGGGAGAATGTGGACCGATGGAGACACAAGCTACAAC
TCCGCCTTCACAAGCAGACTGTCCATCTCTAGGGATACCAGCAAGAGCCAGGTGTTC
CTGAAGATGAATTCTCTGCAGACCGAAGATACAGGAACCTACTACTGCGCCCGGGGC
TATTGGTACTTCGACTTCTGGGGACCTGGAACCATGGTGACTGTGAGCAGCGCTGAG
ACCACAGGCGGCGGCATCGAATTCATGTATCCACCTCCATACCTGGATAACGAAAGGT
CAAATGGAACCATTATCCATATCAAGGAGAAGCACCTGTGTCACACACAGAGTTCCC
CAAAGCTGTTTTGGGCCCTGGTGGTGGTGGCCGGCGTGCTGTTTTGTTACGGGCTGC
TGGTGACCGTTGCTCTGTGCGTCATTTGGACTAATTCCAGGCGCAATTGA
m Ta sequence (GMCSF ss + LC + (G4S)3 + HC + G3 + CD28h + CD28tm + CD28tcy)
SEQ ID NO: 155
MLLLVTSLLLCELPHPAFLLIPDVQMTQSPSYLAASPGESVSIRCKASKSISNNLAWYQEK
PGKANKLLIHSGSTLQSGTPSRFSGSGSGTDFTLTIRSLEFEDFAVYYCQQYNEYPYTFGA
GTKLELKRADAGGGGSGGGGSGGGGSQVQLKESGPGLVQPSQTLSLTCTVSGFSLTTYS
VHWVRQHSGKSLEWMGRMWTDGDTSYNSAFTSRLSISRDTSKSQVFLKMNSLQTEDT
GTYYCARGYWYFDFWGPGTMVTVSSAETTGGGIEFMYPPPYLDNERSNGTIIHIKEKHL
CHTQSSPKLFWALVVVAGVLFCYGLLVTVALCVIWTNSRRN
Anti-hLTBR Heavy Chain Variable Domain (6)
SEQ ID NO: 156
QSVEESGGRLVTPGTPLTLTCTVSGFSLSNYAMSWVRQAPGKGLEWIGTITTGGSTYYAS
WAKGRFTISKTSSTTVDLKMTSLTIEDTATYFCARVDSGWRTGGIWGPGTLVTVSL
Anti-hLTBR Light Chain Variable Domain (6)
SEQ ID NO: 157
AYDMTQTPASVEVAVGGTVTINCQASQNIYNNLAWFQQKPGQPPKLLIYDTSDLASGVP
SRFKGSGSGTEYTLTVSGVECADAATYYCQQGFTASGVDYTFGGGTEVVVK
Anti-hLTBR Heavy Chain Variable Domain (48)
SEQ ID NO: 158
QSLEESGGDLVEPGASLTLTCTASGIDFSSYYYMCWVRQAPGKGLELSACIYDVSGSTW
YASGAKGRFTISKTSSTTVTLQMTSLTAADTATYFCARGYDSGSDYYYYGMDLWGPGTL
VTVSS
Anti-hLTBR Light Chain Variable Domain (48)
SEQ ID NO: 159
DIVMTQTASPVSAAVGDTFTINCQASQSIGSNLAWFQQKPGQPPKLLIYAASNLASGVPSR
FSGSRSGTEYTLTISGVQCEDAATYYCLGTYSSSESAFGGGTELEIL
Anti-hLTBR Heavy Chain Variable Domain (74)
SEQ ID NO: 160
QSLEESGGRLVTPGTPLTLTCTVSGFTISNYLMCWVRQAPGKGLEYIGIINTGSRTYYASW
AKGRFTISKTSTTVDLKITSPTTEDTATYFCARSYGSDIDAIAPWGPGTLVTVSS
Anti-hLTBR Light Chain Variable Domain (74)
SEQ ID NO: 161
FAQVLTQTASSVSAAVGGTVTINCQASQSVYGNNYLSWYQQKPGQPPKLLIYQASVLAS
GVSSRFKGSGSGKQFTLTISGVQCEDAATYYCAGGYVCSSFDCSAFGGGTEVVVK
SEQ ID NO: 162
SKIPPED
SEQ ID NO: 163
SKIPPED
SEQ ID NO: 164
SKIPPED
CD28 hinge (36 amino acids)
SEQ ID NO: 165
IEFMYPPPYLDNERSNGTIIHIKEKHLCHTQSSPK
CD28 hinge (39 amino acids)
SEQ ID NO: 166
IEVMYPPPYLDNEKSNGTIIHVKGKHLCPSPLFPGPSKP
CD8 hinge (48 amino acids)
SEQ ID NO: 167
AKPTTTPAPRPPTPAPTIASQPLSLRPEACRPAAGGAVHTRGLDFACD
CD8 hinge (45 amino acids)
SEQ ID NO: 168
TTTKPVLRTPSPVHPTGTSQPQRPEDCRPRGSVKGTGLDFACDIY
CD28 transmembrane domain (28 amino acids)
SEQ ID NO: 169
LFWALVVVAGVLFCYGLLVTVALCVIWT
CD28 transmembrane domain (27 amino acids)
SEQ ID NO: 170
MFWVLVVVGGVLACYSLLVTVAFIIFWV
CD8 transmembrane domain (22 amino acids)
SEQ ID NO: 171
IWAPLAGICVALLLSLIITLIC
CD8 transmembrane domain (21 amino acids)
SEQ ID NO: 172
IYIWAPLAGTCGVLLLSLVIT
CD8 transmembrane domain (23 amino acids)
SEQ ID NO: 173
IYIWAPLAGTCGVLLLSLVITLY
CD8 transmembrane domain (24 amino acids)
SEQ ID NO: 174
IYIWAPLAGTCGVLLLSLVITLYC
(G4S)3) linker (15 amino acids)
SEQ ID NO: 175
GGGGSGGGGSGGGGS
GGGSSGGGSG linker (10 amino acids)
SEQ ID NO: 176
GGGSSGGGSG
GGSAGGG linker (7 amino acids)
SEQ ID NO: 177
GGSAGGG
GGSAGPAENLYFQGGGGSGGGSG linker (23 amino acids)
SEQ ID NO: 178
GGSAGPAENLYFQGGGGSGGGSG
Anti-mLTBR Heavy Chain Variable Domain (5)
SEQ ID NO: 179
QSLEESGGRLVTPGGSLTLTCTVSGFSLSSRSMGWVRQAPEKGLEWIGIIGGSGDVRYAT
WARGRFTISSTSTTVDLKMTSLTAEDTATYFCGRYNGAGAIDIWGPGTLVTVSF
Anti-mLTBR Light Chain Variable Domain (5)
SEQ ID NO: 180
DPVLTQTPSSVSAVVGGTVTINCQSSQSVYSRGCSWYQQKPGQPPKLLIYQASTLASGVS
SRFSGSGYGTQFALTISGVQCDDAATYYCLGGYSGSIYVFGGGTEVVVK
Anti-mLTBR HC CDR1 (5)
SEQ ID NO: 181
SRSMG
Anti-mLTBR HC CDR2 (5)
SEQ ID NO: 182
IIGGSGDVRYATWA
Anti-mLTBR HC CDR3 (5)
SEQ ID NO: 183
YNGAGAIDI
Anti-mLTBR LC CDR1 (5)
SEQ ID NO: 184
QSSQSVYSRGCS
Anti-mLTBR LC CDR2 (5)
SEQ ID NO: 185
QASTLAS
Anti-mLTBR LC CDR3 (5)
SEQ ID NO: 186
LGGYSGSIYV
Anti-mLTBR Heavy Chain Variable Domain (9)
SEQ ID NO: 187
QSVEESGGRLVTPGTPLTLTCTVSGIDLSSYAMGWVRQAPGEGLEWIGLIDAAGNTYYAT
WAKGRFTISKTSSTTVDLKMTSLTTEDTATYFCARESSSDWYMDIWGPGTLVTVSL
Anti-mLTBR Light Chain Variable Domain (9)
SEQ ID NO: 188
ARVLTQTPSPVSAAVGDTVTINCQASQSVYGANQLSWYQQKPGQPPKLLIYQASKLASG
VPSRLKGSGSGTQFTLTISEVQCDDAATYFCQGYYSGGIYTFGGGTEVVVK
Anti-mLTBR HC CDR1 (9)
SEQ ID NO: 189
SYAMG
Anti-mLTBR HC CDR2 (9)
SEQ ID NO: 190
LIDAAGNTYYATWA
Anti-mLTBR HC CDR3 (9)
SEQ ID NO: 191
ESSSDWYMDI
Anti-mLTBR LC CDR1 (9)
SEQ ID NO: 192
QASQSVYGANQLS
Anti-mLTBR LC CDR2 (9)
SEQ ID NO: 193
QASKLAS
Anti-mLTBR LC CDR3 (9)
SEQ ID NO: 194
QGYYSGGIYT
Anti-mLTBR Heavy Chain Variable Domain (10)
SEQ ID NO: 195
QSLEESGGRLVTPGGSLTLTCTASGSDISPYTISWVRQAPEKGLEWIGIIGKSGGPYYATWA
KGPFTISKTSTTVALKMTSLTAEDTATYFCGRYNGAGAMDIWGPGTLVTVSL
Anti-mLTBR Light Chain Variable Domain (10)
SEQ ID NO: 196
DPVLTQTPSSVFALVGGTVSINCQSSQSVYSMGCSWYQQKPGQPPKLLIYQASTLASGVS
SRFKGSGSGTQFTLTISDVQCDDAATYYCLGGYSGNIYVFGGGTEVVVK
Anti-mLTBR HC CDR1 (10)
SEQ ID NO: 197
PYTIS
Anti-mLTBR HC CDR2 (10)
SEQ ID NO: 198
IIGKSGGPYYATWA
Anti-mLTBR HC CDR3 (10)
SEQ ID NO: 199
YNGAGAMDI
Anti-mLTBR LC CDR1 (10)
SEQ ID NO: 200
QSSQSVYSMGCS
Anti-mLTBR LC CDR2 (10)
SEQ ID NO: 201
QASTLAS
Anti-mLTBR LC CDR3 (10)
SEQ ID NO: 202
LGGYSGNIYV
Anti-mLTBR Heavy Chain Variable Domain (19)
SEQ ID NO: 203
QSVEESGGRLVTPGTPLTLTCTISGFSLSSYDMSWVRQAPGEGLEWIGTISTGGSTYYASW
AKGRFTISKTSSTTVDLKITSPTIEDTATYFCARDRGRSSYYTFGAFDPWGPGTLVTVSS
Anti-mLTBR Light Chain Variable Domain (19)
SEQ ID NO: 204
ALVMTQTPSSTSTAVGDTVTIKCQASQSITSYLSWFQQKPGQPPKLLIYLASTLASGVPSR
FKGSGSGTEFTLTISGVQREDAATYYCLGSYSHSDTAFGGGTELELL
Anti-mLTBR HC CDR1 (19)
SEQ ID NO: 205
SYDMS
Anti-mLTBR HC CDR2 (19)
SEQ ID NO: 206
TISTGGSTYYASWA
Anti-mLTBR HC CDR3 (19)
SEQ ID NO: 207
DRGRSSYYTFGAFDP
Anti-mLTBR LC CDR1 (19)
SEQ ID NO: 208
QASQSITSYLS
Anti-mLTBR LC CDR2 (19)
SEQ ID NO: 209
LASTLAS
Anti-mLTBR LC CDR3 (19)
SEQ ID NO: 210
LGSYSHSDTA
Anti-mLTBR Heavy Chain Variable Domain (62)
SEQ ID NO: 211
QLKESGGGLVQPGGSLKLSCKASGFDFSSYYMSWVRQAPGKGLEWIGWISSGGSTWYA
SWVNGRFTISSHNAQNTLYLQLNSLTAADTATYFCARDRDSSAWGDFNLWGPGTLVTVS
S
Anti-mLTBR Light Chain Variable Domain (62)
SEQ ID NO: 212
DPVMTQTPASVFEPVGGTVTINCQASESISNYLSWYQQKPGQPPKLLIYGASTLEFGVPS
RFSGSGFGTEFSLTISGVQCDDAATYYCQGGYYSSSGTFFAFGGGTEVVVK
Anti-mLTBR HC CDR1 (62)
SEQ ID NO: 213
SYYMS
Anti-mLTBR HC CDR2 (62)
SEQ ID NO: 214
WISSGGSTWYASWVNG
Anti-mLTBR HC CDR3 (62)
SEQ ID NO: 215
DRDSSAWGDFNL
Anti-mLTBR LC CDR1 (62)
SEQ ID NO: 216
QASESISNYLS
Anti-mLTBR LC CDR2 (62)
SEQ ID NO: 217
GASTLEF
Anti-mLTBR LC CDR3 (62)
SEQ ID NO: 218
QGGYYSSSGTFFA
Anti-mLTBR Heavy Chain Variable Domain (63)
SEQ ID NO: 219
QSXEESGGRLVTPGTPLTLTCTASGXSLSNYQMGWVRQAPGEGLEWIGIISSSGSAYYAS
WVNGRFTISKTSTTVDLKVTSLTTEDTATYFCARYLNNDYYDIWGPGTLVTVSL
Anti-mLTBR Light Chain Variable Domain (63)
SEQ ID NO: 220
AQVLTQTPSSVSAAVGGTVTINCQASQSISSYLAWYQQKRGQPPKLLIYYASNLAFGVSS
RFKGSGFGTEYTLTISDLECDDAATYYCQQGYSDINVDNTFGGGTEVVVK
Anti-mLTBR HC CDR1 (63)
SEQ ID NO: 221
NYQMG
Anti-mLTBR HC CDR2 (63)
SEQ ID NO: 222
IISSSGSAYYASWV
Anti-mLTBR HC CDR3 (63)
SEQ ID NO: 223
YLNNDYYDI
Anti-mLTBR LC CDR1 (63)
SEQ ID NO: 224
QASQSISSYLA
Anti-mLTBR LC CDR2 (63)
SEQ ID NO: 225
YASNLAF
Anti-mLTBR LC CDR3 (63)
SEQ ID NO: 226
QQGYSDINVDNT
Anti-mLTBR Heavy Chain Variable Domain (65)
SEQ ID NO: 227
QSXEESGGXLVTPGTPLTLTCTVFGFSLSSNAMSWVRQAPGKGLEWIGFINRYGTTYYAN
WAKGRFTISKTSTTVDLKITSPTIEDTATYFCARADGVEYGYTRIWGPGTLVTVSL
Anti-mLTBR Light Chain Variable Domain (65)
SEQ ID NO: 228
ALVMTQTPSSVFAAVGGTVTINCQASQNIYSNLAWYQQKPGQRPNLLIYEASKMESGVP
SRFSGSGSGPEYTLTISSVQCDDAATYYCQSGAYSASLDNTFGGGTEVVVK
Anti-mLTBR HC CDR1 (65)
SEQ ID NO: 229
SNAMS
Anti-mLTBR HC CDR2 (65)
SEQ ID NO: 230
FINRYGTTYYANWA
Anti-mLTBR HC CDR3 (65)
SEQ ID NO: 231
ADGVEYGYTRI
Anti-mLTBR LC CDR1 (65)
SEQ ID NO: 232
QASQNIYSNLA
Anti-mLTBR LC CDR2 (65)
SEQ ID NO: 233
EASKMES
Anti-mLTBR LC CDR3 (65)
SEQ ID NO: 234
QSGAYSASLDNT
Anti-mLTBR Heavy Chain Variable Domain (66)
SEQ ID NO: 235
QSVEESGGXLVTPGTPLTLTCTIFGFSLSSYDMSWVRQAPGEGLEWIGTISTGGSTYYAS
WGKGRFTISKTSSTTVDLKITSPTMEDTATYFCARDRGRSSYYTFGAFDPWGPGTLVTVS
S
Anti-mLTBR Light Chain Variable Domain (66)
SEQ ID NO: 236
ALVMTQTPSSTSTAVGDTVTIKCQASQSITSYLSWFQQKPGQPPKLLIYLASTLASGVPSR
FKGSGSGTEFTLTISGVQREDAATYYCLGSYSHSDTAFGGGTELELL
Anti-mLTBR HC CDR1 (66)
SEQ ID NO: 237
SYDMS
Anti-mLTBR HC CDR2 (66)
SEQ ID NO: 238
TISTGGSTYYASWG
Anti-mLTBR HC CDR3 (66)
SEQ ID NO: 239
DRGRSSYYTFGAFDP
Anti-mLTBR LC CDR1 (66)
SEQ ID NO: 240
QASQSITSYLS
Anti-mLTBR LC CDR2 (66)
SEQ ID NO: 241
LASTLAS
Anti-mLTBR LC CDR3 (66)
SEQ ID NO: 242
LGSYSHSDTA
Anti-mLTBR Heavy Chain Variable Domain (67)
SEQ ID NO: 243
QSVEESGGRLVTPGTPLTLTCTVSGFSLSRYGMSWVRQAPGKGLEWIGMISSSGSTYNPN
WAKGRFTISKTSTTVDLKITSPTTEDTATYFCAREYWYYFNIWGPGTLVTVSL
Anti-mLTBR Light Chain Variable Domain (67)
SEQ ID NO: 244
AQVLTQTPSSVSAAVGGTVTINCQSSQSVYSTNYLAWYQQKPGQPPKLLIYDASSLASGV
PSRFSGSGSGTQFTLTISGVQCDDAATYYCQGAYYASGWYHTFGGGTEVVVK
Anti-mLTBR HC CDR1 (67)
SEQ ID NO: 245
RYGMS
Anti-mLTBR HC CDR2 (67)
SEQ ID NO: 246
MISSSGSTYNPNWA
Anti-mLTBR HC CDR3 (67)
SEQ ID NO: 247
EYWYYFNI
Anti-mLTBR LC CDR1 (67)
SEQ ID NO: 248
QSSQSVYSTNYLA
Anti-mLTBR LC CDR2 (67)
SEQ ID NO: 249
DASSLAS
Anti-mLTBR LC CDR3 (67)
SEQ ID NO: 250
QGAYYASGWYHT
Anti-mLTBR Heavy Chain Variable Domain (73)
SEQ ID NO: 251
QSGEESGGRLVTPGTPLTLTCTAFGXSLSNYQMGWVRQAPGEGLEWIGIISSSGSAYYAS
WVNGRFTISKTSTTVDLKVTSLTTEDTATYFCARYLNNDYYDIWGPGTLVTVSL
Anti-mLTBR Light Chain Variable Domain (73)
SEQ ID NO: 252
AQVLTQTPSSVSAAVGGTVTINCQASQSISSYLAWYQQKRGQPPKLLIYYASNLASGVSS
RFKGSGFGTEYTLTISDLECDDAATYYCQQGYSDINVDNTFGGGTEVVVK
Anti-mLTBR HC CDR1 (73)
SEQ ID NO: 253
NYQMG
Anti-mLTBR HC CDR2 (73)
SEQ ID NO: 254
IISSSGSAYYASWV
Anti-mLTBR HC CDR3 (73)
SEQ ID NO: 255
YLNNDYYDI
Anti-mLTBR LC CDR1 (73)
SEQ ID NO: 256
QASQSISSYLA
Anti-mLTBR LC CDR2 (73)
SEQ ID NO: 257
YASNLAS
Anti-mLTBR LC CDR3 (73)
SEQ ID NO: 258
QQGYSDINVDNT
Rabbit-Human chimeric sequences for anti-mLTBR Abs with Fc silent mutation
Anti-mLTBR heavy Chain (5)
SEQ ID NO: 259
QSLEESGGRLVTPGGSLTLTCTVSGFSLSSRSMGWVRQAPEKGLEWIGIIGGSGDVRYAT
WARGRFTISSTSTTVDLKMTSLTAEDTATYFCGRYNGAGAIDIWGPGTLVTVSFASTKGPS
VFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPESTRGPSVFLFPPK
PKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVF
SCSVMHEALHNHYTQKSLSLSPGK
Anti-mLTBR Light Chain (5)
SEQ ID NO: 260
DPVLTQTPSSVSAVVGGTVTINCQSSQSVYSRGCSWYQQKPGQPPKLLIYQASTLASGVS
SRFSGSGYGTQFALTISGVQCDDAATYYCLGGYSGSIYVFGGGTEVVVKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC
Anti-mLTBR heavy Chain (9)
SEQ ID NO: 261
QSVEESGGRLVTPGTPLTLTCTVSGIDLSSYAMGWVRQAPGEGLEWIGLIDAAGNTYYAT
WAKGRFTISKTSSTTVDLKMTSLTTEDTATYFCARESSSDWYMDIWGPGTLVTVSLASTK
GPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPESTRGPSVFLF
PPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQ
GNVFSCSVMHEALHNHYTQKSLSLSPGK
Anti-mLTBR Light Chain (9)
SEQ ID NO: 262
ARVLTQTPSPVSAAVGDTVTINCQASQSVYGANQLSWYQQKPGQPPKLLIYQASKLASG
VPSRLKGSGSGTQFTLTISEVQCDDAATYFCQGYYSGGIYTFGGGTEVVVKRTVAAPSVF
IFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
Anti-mLTBR heavy Chain (10)
SEQ ID NO: 263
QSLEESGGRLVTPGGSLTLTCTASGSDISPYTISWVRQAPEKGLEWIGIIGKSGGPYYATWA
KGPFTISKTSTTVALKMTSLTAEDTATYFCGRYNGAGAMDIWGPGTLVTVSLASTKGPSV
FPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPESTRGPSVFLFPPKP
KDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFS
CSVMHEALHNHYTQKSLSLSPGK
Anti-mLTBR Light Chain (10)
SEQ ID NO: 264
DPVLTQTPSSVFALVGGTVSINCQSSQSVYSMGCSWYQQKPGQPPKLLIYQASTLASGVS
SRFKGSGSGTQFTLTISDVQCDDAATYYCLGGYSGNIYVFGGGTEVVVKRTVAAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
Anti-mLTBR heavy Chain (19)
SEQ ID NO: 265
QSVEESGGRLVTPGTPLTLTCTISGFSLSSYDMSWVRQAPGEGLEWIGTISTGGSTYYASW
AKGRFTISKTSSTTVDLKITSPTIEDTATYFCARDRGRSSYYTFGAFDPWGPGTLVTVSSA
STKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPESTRGPS
VFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSR
WQQGNVFSCSVMHEALHNHYTQKSLSLSPGK
Anti-mLTBR Light Chain (19)
SEQ ID NO: 266
ALVMTQTPSSTSTAVGDTVTIKCQASQSITSYLSWFQQKPGQPPKLLIYLASTLASGVPSR
FKGSGSGTEFTLTISGVQREDAATYYCLGSYSHSDTAFGGGTELELLRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
Anti-mLTBR heavy Chain (62)
SEQ ID NO: 267
QLKESGGGLVQPGGSLKLSCKASGFDFSSYYMSWVRQAPGKGLEWIGWISSGGSTWYA
SWVNGRFTISSHNAQNTLYLQLNSLTAADTATYFCARDRDSSAWGDFNLWGPGTLVTVS
SASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPESTRG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKS
RWQQGNVFSCSVMHEALHNHYTQKSLSLSPGK
Anti-mLTBR Light Chain (62)
SEQ ID NO: 268
DPVMTQTPASVFEPVGGTVTINCQASESISNYLSWYQQKPGQPPKLLIYGASTLEFGVPS
RFSGSGFGTEFSLTISGVQCDDAATYYCQGGYYSSSGTFFAFGGGTEVVVKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Anti-mLTBR heavy Chain (63)
SEQ ID NO: 269
QSXEESGGRLVTPGTPLTLTCTASGXSLSNYQMGWVRQAPGEGLEWIGIISSSGSAYYAS
WVNGRFTISKTSTTVDLKVTSLTTEDTATYFCARYLNNDYYDIWGPGTLVTVSLASTKGP
SVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPESTRGPSVFLFPP
KPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGN
VFSCSVMHEALHNHYTQKSLSLSPGK
Anti-mLTBR Light Chain (63)
SEQ ID NO: 270
AQVLTQTPSSVSAAVGGTVTINCQASQSISSYLAWYQQKRGQPPKLLIYYASNLAFGVSS
RFKGSGFGTEYTLTISDLECDDAATYYCQQGYSDINVDNTFGGGTEVVVKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Anti-mLTBR heavy Chain (65)
SEQ ID NO: 271
QSXEESGGXLVTPGTPLTLTCTVFGFSLSSNAMSWVRQAPGKGLEWIGFINRYGTTYYAN
WAKGRFTISKTSTTVDLKITSPTIEDTATYFCARADGVEYGYTRIWGPGTLVTVSLASTKG
PSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPESTRGPSVFLFP
PKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQG
NVFSCSVMHEALHNHYTQKSLSLSPGK
Anti-mLTBR Light Chain (65)
SEQ ID NO: 272
ALVMTQTPSSVFAAVGGTVTINCQASQNIYSNLAWYQQKPGQRPNLLIYEASKMESGVP
SRFSGSGSGPEYTLTISSVQCDDAATYYCQSGAYSASLDNTFGGGTEVVVKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Anti-mLTBR heavy Chain (66)
SEQ ID NO: 273
QSVEESGGXLVTPGTPLTLTCTIFGFSLSSYDMSWVRQAPGEGLEWIGTISTGGSTYYAS
WGKGRFTISKTSSTTVDLKITSPTMEDTATYFCARDRGRSSYYTFGAFDPWGPGTLVTVS
SASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPESTRG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKS
RWQQGNVFSCSVMHEALHNHYTQKSLSLSPGK
Anti-mLTBR Light Chain (66)
SEQ ID NO: 274
ALVMTQTPSSTSTAVGDTVTIKCQASQSITSYLSWFQQKPGQPPKLLIYLASTLASGVPSR
FKGSGSGTEFTLTISGVQREDAATYYCLGSYSHSDTAFGGGTELELLRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
Anti-mLTBR heavy Chain (67)
SEQ ID NO: 275
QSVEESGGRLVTPGTPLTLTCTVSGFSLSRYGMSWVRQAPGKGLEWIGMISSSGSTYNPN
WAKGRFTISKTSTTVDLKITSPTTEDTATYFCAREYWYYFNIWGPGTLVTVSLASTKGPS
VFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPESTRGPSVFLFPPK
PKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVF
SCSVMHEALHNHYTQKSLSLSPGK
Anti-mLTBR Light Chain (67)
SEQ ID NO: 276
AQVLTQTPSSVSAAVGGTVTINCQSSQSVYSTNYLAWYQQKPGQPPKLLIYDASSLASGV
PSRFSGSGSGTQFTLTISGVQCDDAATYYCQGAYYASGWYHTFGGGTEVVVKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
Anti-mLTBR heavy Chain (73)
SEQ ID NO: 277
QSGEESGGRLVTPGTPLTLTCTAFGXSLSNYQMGWVRQAPGEGLEWIGIISSSGSAYYAS
WVNGRFTISKTSTTVDLKVTSLTTEDTATYFCARYLNNDYYDIWGPGTLVTVSLASTKGP
SVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPESTRGPSVFLFPP
KPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGN
VFSCSVMHEALHNHYTQKSLSLSPGK
Anti-mLTBR Light Chain (73)
SEQ ID NO: 278
AQVLTQTPSSVSAAVGGTVTINCQASQSISSYLAWYQQKRGQPPKLLIYYASNLASGVSS
RFKGSGFGTEYTLTISDLECDDAATYYCQQGYSDINVDNTFGGGTEVVVKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Linker
SEQ ID NO: 279
GGGSSGGGSG
IgG4 hinge (S→P)(S228P)
SEQ ID NO: 280
ESKYGPPCPSCP
IgG4 hinge
SEQ ID NO: 281
ESKYGPPCPSCP
IgG4 hinge (S228P) + linker
SEQ ID NO: 282
ESKYGPPCPPCPGGGSSGGGSG
IgG4(HL-CH3) (includes S228P in hinge)
SEQ ID NO: 283
ESKYGPPCPPCPGGGSSGGGSGGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHY
TQKSLSLSLGK
IgG4(L235E, N297Q)
SEQ ID NO: 284
ESKYGPPCPSCPAPEFEGGPSVFLFPPKPKDTLMISRTPEVTCVVVDVSQEDPE
VQFNWYVDGVEVHQAKTKPREEQFSTYRVVSVLTVLHQDWLNGKEYKCK
VSNKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCSV
MHEALHNHYTQKSLSLSLGK
IgG4(S228P, L235E, N297Q)
SEQ ID NO: 285
ESKYGPPCPPCPAPEFEGGPSVFLFPPK PKDTLMISRTPEVTCVVVDVSQEDPE
VQFNWYVDGVEVHQAKTKPREEQFQ STYRVVSVLTVLHQDWLNGKEYKCK
VSNKGLPSSIEKTISKAKGQPREPQVY TLPPSQEEMTKNQVSLTCLVKGFYPS
DIAVEWESNGQPENNYKTTPPVLDSD GSFFLYSRLTVDKSRWQEGNVFSCSV
MHEALHNHYTQKSLSLSLGK
IgG4(CH3)
SEQ ID NO: 286
GQPREPQVYTLPPSQEEMTKNQVSLT CLVKGFYPSDIAVEWESNGQPENNYK
TTPPVLDSDGSFFLYSRLTVDKSRWQ EGNVFSCSVMHEALHNHYTQKSLSLSLGK
CD3zeta transmembrane domain
SEQ ID NO: 287
LCYLLDGILFIYGVILTALFL
CD4 transmembrane domain
SEQ ID NO: 288
MALIVLGGVAGLLLFIGLGIFF
CD8 transmembrane domain (24 amino acids)
SEQ ID NO: 289
IYIWAPLAGTCGVLLLSLVITLYC
41BB transmembrane domain
SEQ ID NO: 290
IISFFLALTSTALLFLLFFLTLRFSVV
CD3zeta instracellular signaling domain
SEQ ID NO: 291
RVKFSRSADAPAYQQGQNQLYNELNLGR REEYDVLDKRRGRDPEMGGKPRRKNPQ
EGLYNELQKDKMAEAYSEIGMKGERRR GKGHDGLYQGLSTATKDTYDALHMQALPPR
CD28 costimulatory domain
SEQ ID NO: 292
RSKRSRLLHSDYMNMTPRRPGPTRKHYQ PYAPPRDFAAYRS
CD28gg* costimulatory domain
SEQ ID NO: 293
RSKRSRQHSDYMNMTPRRPGPTRKHY QPYAPPRDFAAYRS
41BB costimulatory domain
SEQ ID NO: 294
KRGRKKLLYIFKQPFMRPVQTTQEEDGC SCRFPEEEEGGCEL
OX40 costimulatory domain
SEQ ID NO: 295
ALYLLRRDQRLPPDAHKPPGGGSFRTPIQ EEQADAHSTLAKI
5G11 LC Light Chain IGKV16S1*01 IGKJ2-3*01 IGKC*01 SEQ ID NO: 296
SEQ ID NO: 296
DVQMTQSPSYLAASPGESVSIRCKASKSISNNLAWYQEKPGKANKLLIHSGSTLQSGTPS
RFSGSGSGTDFTLTIRSLEFEDFAVYYCQQYNEYPYTFGAGTKLELKRADAAPTVSIFPPS
TEQLATGGASVVCLMNNFYPRDISVKWKIDGTERRDGVLDSVTDQDSKDSTYSMSSTLS
LTKADYESHNLYTCEVVHKTSSSPVVKSFNRNEC
5G11 HC Heavy Chain IGHV2S18*01 IGHJ1*01 IGHG2A*01
SEQ ID NO: 297
QVQLKESGPGLVQPSQTLSLTCTVSGFSLTTYSVHWVRQHSGKSLEWMGRMWTDGDTS
YNSAFTSRLSISRDTSKSQVFLKMNSLQTEDTGTYYCARGYWYFDFWGPGTMVTVSSA
ETTAPSVYPLAPGTALKSNSMVTLGCLVKGYFPEPVTVTWNSGALSSGVHTFPAVLQSGL
YTLTSSVTVPSSTWSSQAVTCNVAHPASSTKVDKKIVPRECNPCGCTGSEVSSVFIFPPKT
KDVLTITLTPKVTCVVVDISQNDPEVRFSWFIDDVEVHTAQTHAPEKQSNSTLRSVSELPI
VHRDWLNGKTFKCKVNSGAFPAPIEKSISKPEGTPRGPQVYTMAPPKEEMTQSQVSITC
MVKGFYPPDIYTEWKMNGQPQENYKNTPPTMDTDGSYFLYSKLNVKKETWQQGNTFT
CSVLHEGLHNHHTEKSLSHSPGK
GMCSF Signal Sequence
SEQ ID NO: 298
MLLLVTSLLLCELPHPAFLLIP

What is claimed is:

1. A lymphotoxin-β receptor antibody comprising a light chain variable region and a heavy chain variable region,
wherein said light chain variable region comprises a CDR L1, a CDR L2, and a CDR L3; and wherein said heavy chain variable region comprises a CDR H1, a CDR H2, and a CDR H3,
wherein the CDR L1, CDR L2, and CDR L3 have amino acid sequences as set forth in SEQ ID NOs: 5, 6, and 7 and wherein the CDR H1, CDR H2, and CDR H3 have amino acid sequences as set forth in SEQ ID NOs: 8, 9 and 10; or
wherein the CDR L1, CDR L2, and CDR L3 have amino acid sequences as set forth in SEQ ID NOs: 13, 14 and 15 and wherein the CDR H1, CDR H2, and CDR H3 have amino acid sequences as set forth in SEQ ID NOs: 16, 17 and 18; or
wherein the CDR L1, CDR L2, and CDR L3 have amino acid sequences as set forth in SEQ ID NOs: 21, 22, and 23 and wherein the CDR H1, CDR H2, and CDR H3 have amino acid sequences as set forth in SEQ ID NOs: 24, 25 and 26; or
wherein the CDR L1, CDR L2, and CDR L3 have amino acid sequences as set forth in SEQ ID NOs: 29, 30, and 31 and wherein the CDR H1, CDR H2, and CDR H3 have amino acid sequences as set forth in SEQ ID NOs: 32, 33 and 34; or
wherein the CDR L1, CDR L2, and CDR L3 have amino acid sequences as set forth in SEQ ID NOs: 37, 38 and 39 and wherein the CDR H1, CDR H2, and CDR H3 have amino acid sequences as set forth in SEQ ID NOs: 40, 41, and 42; or
wherein the CDR L1, CDR L2, and CDR L3 have amino acid sequences as set forth in SEQ ID NOs: 45, 46 and 47 and wherein the CDR H1, CDR H2, and CDR H3 have amino acid sequences as set forth in SEQ ID NOs: 48, 49 and 50; or
wherein the CDR L1, CDR L2, and CDR L3 have amino acid sequences as set forth in SEQ ID NOs: 53, 54 and 55 and wherein the CDR H1, CDR H2, and CDR H3 have amino acid sequences as set forth in SEQ ID NOs: 56, 57 and 58; or
wherein the CDR L1, CDR L2, and CDR L3 have amino acid sequences as set forth in SEQ ID NOs: 61, 62 and 63 and wherein the CDR H1, CDR H2, and CDR H3 have amino acid sequences as set forth in SEQ ID NOs: 64, 65 and 66; or
wherein the CDR L1, CDR L2, and CDR L3 have amino acid sequences as set forth in SEQ ID NOs: 69, 70 and 71 and wherein the CDR H1, CDR H2, and CDR H3 have amino acid sequences as set forth in SEQ ID NOs: 72, 73 and 74; or
wherein the CDR L1, CDR L2, and CDR L3 have amino acid sequences as set forth in SEQ ID NOs: 77, 78 and 79 and wherein the CDR H1, CDR H2, and CDR H3 have amino acid sequences as set forth in SEQ ID NOs: 80, 81 and 82; or
wherein the CDR L1, CDR L2, and CDR L3 have amino acid sequences as set forth in SEQ ID NOs: 85, 86, and 87 and wherein the CDR H1, CDR H2, and CDR H3 have amino acid sequences as set forth in SEQ ID NOs: 88, 89 and 90; or
wherein the CDR L1, CDR L2, and CDR L3 have amino acid sequences as set forth in SEQ ID NOs: 53, 93 and 94 and wherein the CDR H1, CDR H2, and CDR H3 have amino acid sequences as set forth in SEQ ID NOs: 95, 57 and 58; or
wherein the CDR L1, CDR L2, and CDR L3 have amino acid sequences as set forth in SEQ ID NOs: 98, 99 and 100 and wherein the CDR H1, CDR H2, and CDR H3 have amino acid sequences as set forth in SEQ ID NOs: 101, 102 and 10³; or
wherein the CDR L1, CDR L2, and CDR L3 have amino acid sequences as set forth in SEQ ID NOs: 10⁸, 10⁹ and 110 and wherein the CDR H1, CDR H2, and CDR H3 have amino acid sequences as set forth in SEQ ID NOs: 111, 112, and 113; or
wherein the CDR L1, CDR L2, and CDR L3 have amino acid sequences as set forth in SEQ ID NOs: 116, 117 and 118 and wherein the CDR H1, CDR H2, and CDR H3 have amino acid sequences as set forth in SEQ ID NOs: 119, 120 and 121.

2. The antibody of claim 1, wherein the light chain variable region comprises SEQ ID NO:3 and the heavy chain variable region comprises SEQ ID NO:4; or
wherein the light chain variable region comprises SEQ ID NO:11 and the heavy chain variable region comprises SEQ ID NO:12; or
wherein the light chain variable region comprises SEQ ID NO: 19 and the heavy chain variable region comprises SEQ ID NO:20; or
wherein the light chain variable region comprises SEQ ID NO:27 and the heavy chain variable region comprises SEQ ID NO:36; or
wherein the light chain variable region comprises SEQ ID NO:43 and the heavy chain variable region comprises SEQ ID NO:44; or
wherein the light chain variable region comprises SEQ ID NO:51 and the heavy chain variable region comprises SEQ ID NO:52; or
wherein the light chain variable region comprises SEQ ID NO:59 and the heavy chain variable region comprises SEQ ID NO:60; or
wherein the light chain variable region comprises SEQ ID NO:67 and the heavy chain variable region comprises SEQ ID NO:68; or
wherein the light chain variable region comprises SEQ ID NO:75 and the heavy chain variable region comprises SEQ ID NO:76; or
wherein the light chain variable region comprises SEQ ID NO:83 and the heavy chain variable region comprises SEQ ID NO:84; or
wherein the light chain variable region comprises SEQ ID NO:91 and the heavy chain variable region comprises SEQ ID NO:92; or
wherein the light chain variable region comprises SEQ ID NO:96 and the heavy chain variable region comprises SEQ ID NO:97; or
wherein the light chain variable region comprises SEQ ID NO:10⁴ and the heavy chain variable region comprises SEQ ID NO:10⁵; or
wherein the light chain variable region comprises SEQ ID NO: 10⁶ and the heavy chain variable region comprises SEQ ID NO:10⁷; or
wherein the light chain variable region comprises SEQ ID NO:114 and the heavy chain variable region comprises SEQ ID NO:115.

3. The antibody of claim 1, wherein said antibody is a single chain variable fragment (scFv).

4. The antibody of claim 1, wherein said antibody is a humanized antibody.

5. The antibody of claim 1, wherein said antibody is a chimeric antibody.

6. The antibody of claim 5, wherein the antibody comprises a heavy chain and a light chain,
wherein the heavy chain comprises an amino acid sequence set forth in SEQ ID NO:122 and the light chain comprises an animo acid sequence set forth in SEQ ID NO: 123; or
wherein the heavy chain comprises an amino acid sequence set forth in SEQ ID NO:124 and the light chain comprises an animo acid sequence set forth in SEQ ID NO:125; or
wherein the heavy chain comprises an amino acid sequence set forth in SEQ ID NO:126 and the light chain comprises an animo acid sequence set forth in SEQ ID NO:127; or
wherein the heavy chain comprises an amino acid sequence set forth in SEQ ID NO:128 and the light chain comprises an animo acid sequence set forth in SEQ ID NO: 129; or
wherein the heavy chain comprises an amino acid sequence set forth in SEQ ID NO:130 and the light chain comprises an animo acid sequence set forth in SEQ ID NO:131; or
wherein the heavy chain comprises an amino acid sequence set forth in SEQ ID NO: 132 and the light chain comprises an animo acid sequence set forth in SEQ ID NO:133; or
wherein the heavy chain comprises an amino acid sequence set forth in SEQ ID NO: 134 and the light chain comprises an animo acid sequence set forth in SEQ ID NO: 135; or
wherein the heavy chain comprises an amino acid sequence set forth in SEQ ID NO:136 and the light chain comprises an animo acid sequence set forth in SEQ ID NO:137; or
wherein the heavy chain comprises an amino acid sequence set forth in SEQ ID NO:138 and the light chain comprises an animo acid sequence set forth in SEQ ID NO: 139; or
wherein the heavy chain comprises an amino acid sequence set forth in SEQ ID NO:140 and the light chain comprises an animo acid sequence set forth in SEQ ID NO: 141; or
wherein the heavy chain comprises an amino acid sequence set forth in SEQ ID NO:142 and the light chain comprises an animo acid sequence set forth in SEQ ID NO: 143; or
wherein the heavy chain comprises an amino acid sequence set forth in SEQ ID NO:144 and the light chain comprises an animo acid sequence set forth in SEQ ID NO:145; or
wherein the heavy chain comprises an amino acid sequence set forth in SEQ ID NO:146 and the light chain comprises an animo acid sequence set forth in SEQ ID NO: 147; or
wherein the heavy chain comprises an amino acid sequence set forth in SEQ ID NO:148 and the light chain comprises an animo acid sequence set forth in SEQ ID NO:149; or
wherein the heavy chain comprises an amino acid sequence set forth in SEQ ID NO:150 and the light chain comprises an animo acid sequence set forth in SEQ ID NO: 151; or
wherein the heavy chain comprises an amino acid sequence set forth in SEQ ID NO:152 and the light chain comprises an animo acid sequence set forth in SEQ ID NO:153.

7. A single chain variable fragment (scFv) that binds a lymphotoxin-β receptor comprising a light chain variable region and a heavy chain variable region,
wherein said light chain variable region comprises a CDR L1, a CDR L2, and a CDR L3; and wherein said heavy chain variable region comprises a CDR H1, a CDR H2, and a CDR H3,
wherein the CDR L1, CDR L2, and CDR L3 have amino acid sequences as set forth in SEQ ID NOs: 5, 6, and 7 and wherein the CDR H1, CDR H2, and CDR H3 have amino acid sequences as set forth in SEQ ID NOs: 8, 9 and 10; or
wherein the CDR L1, CDR L2, and CDR L3 have amino acid sequences as set forth in SEQ ID NOs: 13, 14 and 15 and wherein the CDR H1, CDR H2, and CDR H3 have amino acid sequences as set forth in SEQ ID NOs: 16, 17 and 18; or
wherein the CDR L1, CDR L2, and CDR L3 have amino acid sequences as set forth in SEQ ID NOs: 21, 22, and 23 and wherein the CDR H1, CDR H2, and CDR H3 have amino acid sequences as set forth in SEQ ID NOs: 24, 25 and 26; or
wherein the CDR L1, CDR L2, and CDR L3 have amino acid sequences as set forth in SEQ ID NOs: 29, 30, and 31 and wherein the CDR H1, CDR H2, and CDR H3 have amino acid sequences as set forth in SEQ ID NOs: 32, 33 and 34; or
wherein the CDR L1, CDR L2, and CDR L3 have amino acid sequences as set forth in SEQ ID NOs: 37, 38 and 39 and wherein the CDR H1, CDR H2, and CDR H3 have amino acid sequences as set forth in SEQ ID NOs: 40, 41, and 42; or
wherein the CDR L1, CDR L2, and CDR L3 have amino acid sequences as set forth in SEQ ID NOs: 45, 46 and 47 and wherein the CDR H1, CDR H2, and CDR H3 have amino acid sequences as set forth in SEQ ID NOs: 48, 49 and 50; or
wherein the CDR L1, CDR L2, and CDR L3 have amino acid sequences as set forth in SEQ ID NOs: 53, 54 and 55 and wherein the CDR H1, CDR H2, and CDR H3 have amino acid sequences as set forth in SEQ ID NOs: 56, 57 and 58; or
wherein the CDR L1, CDR L2, and CDR L3 have amino acid sequences as set forth in SEQ ID NOs: 61, 62 and 63 and wherein the CDR H1, CDR H2, and CDR H3 have amino acid sequences as set forth in SEQ ID NOs: 64, 65 and 66; or
wherein the CDR L1, CDR L2, and CDR L3 have amino acid sequences as set forth in SEQ ID NOs: 69, 70 and 71 and wherein the CDR H1, CDR H2, and CDR H3 have amino acid sequences as set forth in SEQ ID NOs: 72, 73 and 74; or
wherein the CDR L1, CDR L2, and CDR L3 have amino acid sequences as set forth in SEQ ID NOs: 77, 78 and 79 and wherein the CDR H1, CDR H2, and CDR H3 have amino acid sequences as set forth in SEQ ID NOs: 80, 81 and 82; or
wherein the CDR L1, CDR L2, and CDR L3 have amino acid sequences as set forth in SEQ ID NOs: 85, 86, and 87 and wherein the CDR H1, CDR H2, and CDR H3 have amino acid sequences as set forth in SEQ ID NOs: 88, 89 and 90; or
wherein the CDR L1, CDR L2, and CDR L3 have amino acid sequences as set forth in SEQ ID NOs: 53, 93 and 94 and wherein the CDR H1, CDR H2, and CDR H3 have amino acid sequences as set forth in SEQ ID NOs: 95, 57 and 58; or
wherein the CDR L1, CDR L2, and CDR L3 have amino acid sequences as set forth in SEQ ID NOs: 98, 99 and 100 and wherein the CDR H1, CDR H2, and CDR H3 have amino acid sequences as set forth in SEQ ID NOs: 101, 102 and 10³; or
wherein the CDR L1, CDR L2, and CDR L3 have amino acid sequences as set forth in SEQ ID NOs: 10⁸, 10⁹ and 110 and wherein the CDR H1, CDR H2, and CDR H3 have amino acid sequences as set forth in SEQ ID NOs: 111, 112, and 113; or
wherein the CDR L1, CDR L2, and CDR L3 have amino acid sequences as set forth in SEQ ID NOs: 116, 117 and 118 and wherein the CDR H1, CDR H2, and CDR H3 have amino acid sequences as set forth in SEQ ID NOs: 119, 120 and 121.

8. The scFV of claim 1, wherein the light chain variable region comprises SEQ ID NO:3 and the heavy chain variable region comprises SEQ ID NO:4; or
wherein the light chain variable region comprises SEQ ID NO:11 and the heavy chain variable region comprises SEQ ID NO:12; or
wherein the light chain variable region comprises SEQ ID NO: 19 and the heavy chain variable region comprises SEQ ID NO:20; or
wherein the light chain variable region comprises SEQ ID NO:27 and the heavy chain variable region comprises SEQ ID NO:36; or
wherein the light chain variable region comprises SEQ ID NO:43 and the heavy chain variable region comprises SEQ ID NO:44; or
wherein the light chain variable region comprises SEQ ID NO:51 and the heavy chain variable region comprises SEQ ID NO:52; or
wherein the light chain variable region comprises SEQ ID NO:59 and the heavy chain variable region comprises SEQ ID NO:60; or
wherein the light chain variable region comprises SEQ ID NO:67 and the heavy chain variable region comprises SEQ ID NO:68; or
wherein the light chain variable region comprises SEQ ID NO:75 and the heavy chain variable region comprises SEQ ID NO:76; or
wherein the light chain variable region comprises SEQ ID NO:83 and the heavy chain variable region comprises SEQ ID NO:84; or
wherein the light chain variable region comprises SEQ ID NO:91 and the heavy chain variable region comprises SEQ ID NO:92; or
wherein the light chain variable region comprises SEQ ID NO:96 and the heavy chain variable region comprises SEQ ID NO:97; or
wherein the light chain variable region comprises SEQ ID NO:10⁴ and the heavy chain variable region comprises SEQ ID NO:10⁵; or
wherein the light chain variable region comprises SEQ ID NO:10⁶ and the heavy chain variable region comprises SEQ ID NO:10⁷; or
wherein the light chain variable region comprises SEQ ID NO:114 and the heavy chain variable region comprises SEQ ID NO:115.

9. The scFv of claim 7, wherein the scFv further comprises a hinge domain.

10. The scFv of claim 9, wherein the hinge domain is a CD28 hinge domain or a CD8 hinge domain.

11. The scFv of claim 7, wherein the scFv further comprises a transmembrane domain.

12. The scFv of claim 11, wherein the transmembrane domain is a CD28 transmembrane domain or a CD8 transmembrane domain.

13. The scFv of claim 7, wherein the scFv comprises a linker sequence.

14. A nucleic acid molecule comprising a nucleic acid molecule encoding the antibody of claim 1.

15. A nucleic acid molecule comprising a nucleic acid molecule encoding the scFv of claim 7.

16. A vector comprising the nucleic acid molecule of claim 14.

17. A vector comprising the nucleic acid molecule of claim 15.

18. A pharmaceutical composition comprising the antibody of claim 1 and a pharmaceutically acceptable excipient.

19. A pharmaceutical composition comprising the scFv of claim 7 and a pharmaceutically acceptable excipient.

20. A method of treating cancer in a subject in need thereof comprising administering to a subject a therapeutically effective amount of the pharmaceutical composition of claim 18, thereby treating cancer in the subject.

21. The method of claim 20, wherein the cancer is a brain cancer.

22. The method of claim 20, wherein the method further comprises administering to the subject a second therapeutic agent.

23. The method of claim 22, wherein the second therapeutic agent is an immune checkpoint inhibitor.

24. A population of human T cells comprising the scFv of claim 7.

25. The population of human T cells of claim 24, wherein the population of human T cells further comprises a chimeric antigen receptor (CAR).

26. The population of human T cells of claim 25, wherein the CAR comprises a HER2 targeting sequence, a CD19 targeting sequence or an Interleukin Receptor targeting sequence.

27. The population of human T cells of claim 25, wherein the chimeric antigen receptor (CAR) comprises an interleukin.

28. The population of human T cells of claim 27, wherein the interleukin is IL13.

29. A method of treating cancer in a subject in need thereof comprising administering to a subject a therapeutically effective amount of a composition comprising the population of human T cells of claim 24, thereby treating cancer in the subject.

30. The method of claim 29, wherein the cancer is a brain cancer.

31. A method of making a population of T cells expressing a membrane bound scFv targeted to a lymphotoxin-β receptor comprising
(i) obtaining a population of T cells from a subject;
(ii) transducing the population of T cells with a nucleic acid sequence encoding the scFv of claim 7; and
(iii) isolating the population of T cells expressing the membrane bound scFv targeted to the lymphotoxin-β receptor.

32. The method of claim 31, wherein the method further comprises modifying the population of T cells to express a chimeric antigen receptor (CAR).

33. The method of claim 32, wherein the CAR comprises a HER2 targeting sequence, a CD19 targeting sequence or an Interleukin Receptor targeting sequence.

34. The method of claim 32, wherein the chimeric antigen receptor (CAR) comprises an interleukin.

35. The method of claim 34, wherein the interleukin is IL13.

36. A population of T cells expressing the membrane bound scFv targeted to a lymphotoxin-β receptor made according to the method of claim 31.
